SEQUENCE LISTING (47 sequences)

SEQ ID NO: 1 - DNA, 1725 bp, Respiratory Syncytial Virus
atggagttgccaatcctcaaagcaaatgcaattaccacaatcctcgctgcagtcacattttgctttgcttctagtcaaaacatcactgaagaattttatcaatcaacatgcagtgcagttagcaaaggctatcttagtgctctaagaactggttggtatactagtgttataactatagaattaagtaatatcaaggaaaataagtgtaatggaacagatgctaaggtaaaattgataaaccaagaattagataaatataaaaatgctgtaacagaattgcagttgctcatgcaaagcacaacagcagcaaacaatcgagccagaagagaactaccaaggtttatgaattatacactcaacaataccaaaaaaaccaatgtaacattaagcaagaaaaggaaaagaagatttcttggttttttgttaggtgttggatctgcaatcgccagtggcattgctgtatctaaggtcctgcacttagaaggagaagtgaacaagatcaaaagtgctctactatccacaaacaaggccgtagtcagcttatcaaatggagttagtgtcttaaccagcaaagtgttagacctcaaaaactatatagataaacaattgttacctattgtgaataagcaaagctgcagaatatcaaatatagaaactgtgatagagttccaacaaaagaacaacagactactagagattaccagggaatttagtgttaatgcaggtgtaactacacctgtaagcacttacatgttaactaatagtgaattattgtcattaatcaatgatatgcctataacaaatgatcagaaaaagttaatgtccaacaatgttcaaatagttagacagcaaagttactctatcatgtccataataaaagaggaagtcttagcatatgtagtacaattaccactatatggtgtgatagatacaccttgttggaaattacacacatcccctctatgtacaaccaacacaaaagaagggtcaaacatctgtttaacaagaactgacagaggatggtactgtgacaatgcaggatcagtatctttcttcccacaagctgaaacatgtaaagttcaatcgaatcgagtattttgtgacacaatgaacagtttaacattaccaagtgaagtaaatctctgcaatgttgacatattcaatcccaaatatgattgtaaaattatgacttcaaaaacagatgtaagcagctccgttatcacatctctaggagccattgtgtcatgctatggcaaaactaaatgtacagcatccaataaaaatcgtggaatcataaagacattttctaacgggtgtgattatgtatcaaataaaggggtggacactgtgtctgtaggtaacacattatattatgtaaataagcaagaaggcaaaagtctctatgtaaaaggtgaaccaataataaatttctatgacccattagtattcccctctgatgaatttgatgcatcaatatctcaagtcaatgagaagattaaccagagtttagcatttattcgtaaatccgatgaattattacatcatgtaaatgctggtaaatcaaccacaaatatcatgataactactataattatagtgattatagtaatattgttatcattaattgctgttggactgctcctatactgtaaggccagaagcacaccagtcacactaagcaaggatcaactgagtggtataaataatattgcatttagtaactga

SEQ ID NO: 2 - protein, 574 aa, Respiratory Syncytial Virus
MELPILKANAITTILAAVTFCFASSQNITEEFYQSTCSAVSKGYLSALRTGWYTSVITIELSNIKENKCNGTDAKVKLINQELDKYKNAVTELQLLMQSTTAANNRARRELPRFMNYTLNNTKKTNVTLSKKRKRRFLGFLLGVGSAIASGIAVSKVLHLEGEVNKIKSALLSTNKAVVSLSNGVSVLTSKVLDLKNYIDKQLLPIVNKQSCRISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHHVNAGKSTTNIMITTIIIVIIVILLSLIAVGLLLYCKARSTPVTLSKDQLSGINNIAFSN

SEQ ID NO: 3 - DNA, 897 bp, Respiratory Syncytial Virus
atgtccaaaaacaaggaccaacgcaccgctaagacactagaaaagacctgggacactctcaatcatttattattcatatcatcgggcttatataagttaaatcttaaatctatagcacaaatcacattatccattctggcaatgataatctcaacttcacttataattacagccatcatattcatagcctcggcaaaccacaaagtcacactaacaactgcaatcatacaagatgcaacaagccagatcaagaacacaaccccaacatacctcactcaggatcctcagcttggaatcagcttctccaatctgtctgaaattacatcacaaaccaccaccatactagcttcaacaacaccaggagtcaagtcaaacctgcaacccacaacagtcaagactaaaaacacaacaacaacccaaacacaacccagcaagcccactacaaaacaacgccaaaacaaaccaccaaacaaacccaataatgattttcacttcgaagtgtttaactttgtaccctgcagcatatgcagcaacaatccaacctgctgggctatctgcaaaagaataccaaacaaaaaaccaggaaagaaaaccaccaccaagcctacaaaaaaaccaaccttcaagacaaccaaaaaagatctcaaacctcaaaccactaaaccaaaggaagtacccaccaccaagcccacagaagagccaaccatcaacaccaccaaaacaaacatcacaactacactgctcaccaacaacaccacaggaaatccaaaactcacaagtcaaatggaaaccttccactcaacctcctccgaaggcaatctaagcccttctcaagtctccacaacatccgagcacccatcacaaccctcatctccacccaacacaacacgccagtag

SEQ ID NO: 4 - protein, 298 aa, Respiratory Syncytial Virus
MSKNKDQRTAKTLEKTWDTLNHLLFISSGLYKLNLKSIAQITLSILAMIISTSLIITAIIFIASANHKVTLTTAIIQDATSQIKNTTPTYLTQDPQLGISFSNLSEITSQTTTILASTTPGVKSNLQPTTVKTKNTTTTQTQPSKPTTKQRQNKPPNKPNNDFHFEVFNFVPCSICSNNPTCWAICKRIPNKKPGKKTTTKPTKKPTFKTTKKDLKPQTTKPKEVPTTKPTEEPTINTTKTNITTTLLTNNTTGNPKLTSQMETFHSTSSEGNLSPSQVSTTSEHPSQPSSPPNTTRQ

SEQ ID NO: 5 - DNA, 1812 bp, Artificial Sequence
  Recombinant F2GF1-1 Chimeric Polynucleotide
atgggccatcatcatcatcatcatcatcatcatcacagcagcggccatatcgacgacgacgacaagcatatgctcgagagtagccaaaacatcactgaagaattttatcaatcaacatgcagtgcagttagcaaaggctatcttagtgctctaagaactggttggtatactagtgttataactatagaattaagtaatatcaaggaaaataagtgtaatggaacagatgctaaggtaaaattgataaaacaagaattagataaatataaaaatgctgtaacagaattgcagttgctcatgcaaagcacaccagcagcaaacaatcgagccagaagagaactaccaaggtttatgaattatacactcaacaataccaaaaaaaccaatgtaacattaagcggcggtcccacaacagtcaagactaaaaacacaacaacaacccaaacacaacccagcaagcccactacaaaacaacgccaaaacaaaccaccaaacaaacccaataatgattttcacttcgaagtgtttaactttgtaccctgcagcatctgcagcaacaatccaacctgctgggctatctgcaaaagaataccagctaaaaaaccaggaaagaaaaccaccaccaagcctacaaaaaaaccaaccttcaagacaaccaaaaaagatctcaaacctcaaaccactaaaccaaaggaagtacccaccaccaagggtggcgaaggagaagtgaacaagatcaaaagtgctctactatccacaaacaaggccgtagtcagcttatcaaatggagttagtgtcttaaccagcaaagtgttagacctcaaaaactatatagataaacaattgttacctattgtgaataagcaaagctgcagaatatcaaatatagaaactgtgatagagttccaacaaaagaacaacagactactagagattaccagggaatttagtgttaatgcaggtgtaactacacctgtaagcacttacatgttaactaatagtgaattattgtcattaatcaatgatatgcctataacaaatgatcagaaaaagttaatgtccaacaatgttcaaatagttagacagcaaagttactctatcatgtccataataaaagaggaagtcttagcatatgtagtacaattaccactatatggtgtgatagatacaccttgttggaaattacacacatcccctctatgtacaaccaacacaaaagaagggtcaaacatctgtttaacaagaactgacagaggatggtactgtgacaatgcaggatcagtatctttcttcccacaagctgaaacatgtaaagttcaatcgaatcgagtattttgtgacacaatgaacagtttaacattaccaagtgaagtaaatctctgcaatgttgacatattcaatcccaaatatgattgtaaaattatgacttcaaaaacagatgtaagcagctccgttatcacatctctaggagccattgtgtcatgctatggcaaaactaaatgtacagcatccaataaaaatcgtggaatcataaagacattttctaacgggtgtgattatgtatcaaataaaggggtggacactgtgtctgtaggtaacacattatattatgtaaataagcaagaaggcaaaagtctctatgtaaaaggtgaaccaataataaatttctatgacccattagtattcccctctgatgaatttgatgcatcaatatctcaagtcaatgagaagattaaccagagtttagcatttattcgtaaatccgatgaattattacataatgtaaatgctggtaaatcaaccacaaattga

SEQ ID NO: 6 - protein, 603 aa, Artificial Sequence
  Recombinant F2GF1-1 Chimeric Polypeptide
MGHHHHHHHHHHSSGHIDDDDKHMLESSQNITEEFYQSTCSAVSKGYLSALRTGWYTSVITIELSNIKENKCNGTDAKVKLIKQELDKYKNAVTELQLLMQSTPAANNRARRELPRFMNYTLNNTKKTNVTLSGGPTTVKTKNTTTTQTQPSKPTTKQRQNKPPNKPNNDFHFEVFNFVPCSICSNNPTCWAICKRIPAKKPGKKTTTKPTKKPTFKTTKKDLKPQTTKPKEVPTTKGGEGEVNKIKSALLSTNKAVVSLSNGVSVLTSKVLDLKNYIDKQLLPIVNKQSCRISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTN

SEQ ID NO: 7 - DNA, 1680 bp, Artificial Sequence
  Recombinant F2GF1-2 Chimeric Polynucleotide
atgggccatcatcatcatcatcatcatcatcatcacagcagcggccatatcgacgacgacgacaagcatatgctcgagagtagccaaaacatcactgaagaattttatcaatcaacatgcagtgcagttagcaaaggctatcttagtgctctaagaactggttggtatactagtgttataactatagaattaagtaatatcaaggaaaataagtgtaatggaacagatgctaaggtaaaattgataaaacaagaattagataaatataaaaatgctgtaacagaattgcagttgctcatgcaaagcacaccagcagcaaacaatcgagccggcggtaaacaacgccaaaacaaaccaccaaacaaacccaataatgattttcacttcgaagtgtttaactttgtaccctgcagcatctgcagcaacaatccaacctgctgggctatctgcaaaagaataccagctaaaaaaccaggaaagaaaaccaccaccaagcctacaaaaaaaccaaccttcaagacaaccaaaaaagatctcaaacctcaaaccactaaaccaaaggaagtacccaccaccaagggtggcgaaggagaagtgaacaagatcaaaagtgctctactatccacaaacaaggccgtagtcagcttatcaaatggagttagtgtcttaaccagcaaagtgttagacctcaaaaactatatagataaacaattgttacctattgtgaataagcaaagctgcagaatatcaaatatagaaactgtgatagagttccaacaaaagaacaacagactactagagattaccagggaatttagtgttaatgcaggtgtaactacacctgtaagcacttacatgttaactaatagtgaattattgtcattaatcaatgatatgcctataacaaatgatcagaaaaagttaatgtccaacaatgttcaaatagttagacagcaaagttactctatcatgtccataataaaagaggaagtcttagcatatgtagtacaattaccactatatggtgtgatagatacaccttgttggaaattacacacatcccctctatgtacaaccaacacaaaagaagggtcaaacatctgtttaacaagaactgacagaggatggtactgtgacaatgcaggatcagtatctttcttcccacaagctgaaacatgtaaagttcaatcgaatcgagtattttgtgacacaatgaacagtttaacattaccaagtgaagtaaatctctgcaatgttgacatattcaatcccaaatatgattgtaaaattatgacttcaaaaacagatgtaagcagctccgttatcacatctctaggagccattgtgtcatgctatggcaaaactaaatgtacagcatccaataaaaatcgtggaatcataaagacattttctaacgggtgtgattatgtatcaaataaaggggtggacactgtgtctgtaggtaacacattatattatgtaaataagcaagaaggcaaaagtctctatgtaaaaggtgaaccaataataaatttctatgacccattagtattcccctctgatgaatttgatgcatcaatatctcaagtcaatgagaagattaaccagagtttagcatttattcgtaaatccgatgaattattacataatgtaaatgctggtaaatcaaccacaaattga

SEQ ID NO: 8 - protein, 559 aa, Artificial Sequence
  Recombinant F2GF1-2 Chimeric Polypeptide
MGHHHHHHHHHHSSGHIDDDDKHMLESSQNITEEFYQSTCSAVSKGYLSALRTGWYTSVITIELSNIKENKCNGTDAKVKLIKQELDKYKNAVTELQLLMQSTPAANNRAGGKQRQNKPPNKPNNDFHFEVFNFVPCSICSNNPTCWAICKRIPAKKPGKKTTTKPTKKPTFKTTKKDLKPQTTKPKEVPTTKGGEGEVNKIKSALLSTNKAVVSLSNGVSVLTSKVLDLKNYIDKQLLPIVNKQSCRISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTN

SEQ ID NO: 9 - DNA, 1743 bp, Artificial Sequence
  Recombinant F2GF1-3 Chimeric Polynucleotide
atgggccatcatcatcatcatcatcatcatcatcacagcagcggccatatcgacgacgacgacaagcatatgctcgagagtagccaaaacatcactgaagaattttatcaatcaacatgcagtgcagttagcaaaggctatcttagtgctctaagaactggttggtatactagtgttataactatagaattaagtaatatcaaggaaaataagtgtaatggaacagatgctaaggtaaaattgatgaaacaagaattagataaatataaaaatgctgtaacagaattgcagttgctcatgcaaagcacaccagcagcaaacaatcgagccggcggtcccacaacagtcaagactaaaaacacaacaacaacccaaacacaacccagcaagcccactacaaaacaacgccaaaacaaaccaccaaacaaacccaataatgattttcacttcgaagtgtttaactttgtaccctgcagcatctgcagcaacaatccaacctgctgggctatctgcaaaagaataccagctaaaaaaccaggaaagaaaaccaccaccaagcctacaaaaaaaccaaccttcaagacaaccaaaaaagatctcaaacctcaaaccactaaaccaaaggaagtacccaccaccaagggtggcgaaggagaagtgaacaagatcaaaagtgctctactatccacaaacaaggccgtagtcagcttatcaaatggagttagtgtcttaaccagcaaagtgttagacctcaaaaactatatagataaacaattgttacctattgtgaataagcaaagctgcagaatatcaaatatagaaactgtgatagagttccaacaaaagaacaacagactactagagattaccagggaatttagtgttaatgcaggtgtaactacacctgtaagcacttacatgttaactaatagtgaattattgtcattaatcaatgatatgcctataacaaatgatcagaaaaagttaatgtccaacaatgttcaaatagttagacagcaaagttactctatcatgtccataataaaagaggaagtcttagcatatgtagtacaattaccactatatggtgtgatagatacaccttgttggaaattacacacatcccctctatgtacaaccaacacaaaagaagggtcaaacatctgtttaacaagaactgacagaggatggtactgtgacaatgcaggatcagtatctttcttcccacaagctgaaacatgtaaagttcaatcgaatcgagtattttgtgacacaatgaacagtttaacattaccaagtgaagtaaatctctgcaatgttgacatattcaatcccaaatatgattgtaaaattatgacttcaaaaacagatgtaagcagctccgttatcacatctctaggagccattgtgtcatgctatggcaaaactaaatgtacagcatccaataaaaatcgtggaatcataaagacattttctaacgggtgtgattatgtatcaaataaaggggtggacactgtgtctgtaggtaacacattatattatgtaaataagcaagaaggcaaaagtctctatgtaaaaggtgaaccaataataaatttctatgacccattagtattcccctctgatgaatttgatgcatcaatatctcaagtcaatgagaagattaaccagagtttagcatttattcgtaaatccgatgaattattacataatgtaaatgctggtaaatcaaccacaaattga

SEQ ID NO: 10 - protein, 580 aa, Artificial Sequence
  Recombinant F2GF1-3 Chimeric Polypeptide
MGHHHHHHHHHHSSGHIDDDDKHMLESSQNITEEFYQSTCSAVSKGYLSALRTGWYTSVITIELSNIKENKCNGTDAKVKLMKQELDKYKNAVTELQLLMQSTPAANNRAGGPTTVKTKNTTTTQTQPSKPTTKQRQNKPPNKPNNDFHFEVFNFVPCSICSNNPTCWAICKRIPAKKPGKKTTTKPTKKPTFKTTKKDLKPQTTKPKEVPTTKGGEGEVNKIKSALLSTNKAVVSLSNGVSVLTSKVLDLKNYIDKQLLPIVNKQSCRISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTN

SEQ ID NO: 11 - DNA, 1749 bp, Artificial Sequence
  Recombinant F2GF1-4 Chimeric Polynucleotide
atgggccatcatcatcatcatcatcatcatcatcacagcagcggccatatcgacgacgacgacaagcatatgctcgagagtagccaaaacatcactgaagaattttatcaatcaacatgcagtgcagttagcaaaggctatcttagtgctctaagaactggttggtatactagtgttataactatagaattaagtaatatcaaggaaaataagtgtaatggaacagatgctaaggtaaaattgatgaaacaagaattagataaatataaaaatgctgtaacagaattgcagttgctcatgcaaagcacaccagcagcaaacaatcgagccagaagagaactaccaaggtttatgaattatacactcaacaataccaaaaaaaccaatgtaacattaagcggcggtaaacaacgccaaaacaaaccaccaaacaaacccaataatgattttcacttcgaagtgtttaactttgtaccctgcagcatctgcagcaacaatccaacctgctgggctatctgcaaaagaataccagctaaaaaaccaggaaagaaaaccaccaccaagcctacaaaaaaaccaaccttcaagacaaccaaaaaagatctcaaacctcaaaccactaaaccaaaggaagtacccaccaccaagggtggcgaaggagaagtgaacaagatcaaaagtgctctactatccacaaacaaggccgtagtcagcttatcaaatggagttagtgtcttaaccagcaaagtgttagacctcaaaaactatatagataaacaattgttacctattgtgaataagcaaagctgcagaatatcaaatatagaaactgtgatagagttccaacaaaagaacaacagactactagagattaccagggaatttagtgttaatgcaggtgtaactacacctgtaagcacttacatgttaactaatagtgaattattgtcattaatcaatgatatgcctataacaaatgatcagaaaaagttaatgtccaacaatgttcaaatagttagacagcaaagttactctatcatgtccataataaaagaggaagtcttagcatatgtagtacaattaccactatatggtgtgatagatacaccttgttggaaattacacacatcccctctatgtacaaccaacacaaaagaagggtcaaacatctgtttaacaagaactgacagaggatggtactgtgacaatgcaggatcagtatctttcttcccacaagctgaaacatgtaaagttcaatcgaatcgagtattttgtgacacaatgaacagtttaacattaccaagtgaagtaaatctctgcaatgttgacatattcaatcccaaatatgattgtaaaattatgacttcaaaaacagatgtaagcagctccgttatcacatctctaggagccattgtgtcatgctatggcaaaactaaatgtacagcatccaataaaaatcgtggaatcataaagacattttctaacgggtgtgattatgtatcaaataaaggggtggacactgtgtctgtaggtaacacattatattatgtaaataagcaagaaggcaaaagtctctatgtaaaaggtgaaccaataataaatttctatgacccattagtattcccctctgatgaatttgatgcatcaatatctcaagtcaatgagaagattaaccagagtttagcatttattcgtaaatccgatgaattattacataatgtaaatgctggtaaatcaaccacaaattga

SEQ ID NO: 12 - protein, 582 aa, Artificial Sequence
  Recombinant F2GF1-4 Chimeric Polypeptide
MGHHHHHHHHHHSSGHIDDDDKHMLESSQNITEEFYQSTCSAVSKGYLSALRTGWYTSVITIELSNIKENKCNGTDAKVKLMKQELDKYKNAVTELQLLMQSTPAANNRARRELPRFMNYTLNNTKKTNVTLSGGKQRQNKPPNKPNNDFHFEVFNFVPCSICSNNPTCWAICKRIPAKKPGKKTTTKPTKKPTFKTTKKDLKPQTTKPKEVPTTKGGEGEVNKIKSALLSTNKAVVSLSNGVSVLTSKVLDLKNYIDKQLLPIVNKQSCRISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTN

SEQ ID NO: 13 - DNA, 1812 bp, Artificial Sequence
  Recombinant F2GF1-5 Chimeric Polynucleotide
atgggccatcatcatcatcatcatcatcatcatcacagcagcggccatatcgacgacgacgacaagcatatgctcgagagtagccaaaacatcactgaagaattttatcaatcaacatgcagtgcagttagcaaaggctatcttagtgctctaagaactggttggtatactagtgttataactatagaattaagtaatatcaaggaaaataagtgtaatggaacagatgctaaggtaaaattgatgaaacaagaattagataaatataaaaatgctgtaacagaattgcagttgctcatgcaaagcacaccagcagcaaacaatcgagccagaagagaactaccaaggtttatgaattatacactcaacaataccaaaaaaaccaatgtaacattaagcggcggtcccacaacagtcaagactaaaaacacaacaacaacccaaacacaacccagcaagcccactacaaaacaacgccaaaacaaaccaccaaacaaacccaataatgattttcacttcgaagtgtttaactttgtaccctgcagcatctgcagcaacaatccaacctgctgggctatctgcaaaagaataccaaacaaaaaaccaggaaagaaaaccaccaccaagcctacaaaaaaaccaaccttcaagacaaccaaaaaagatctcaaacctcaaaccactaaaccaaaggaagtacccaccaccaagggtggcgaaggagaagtgaacaagatcaaaagtgctctactatccacaaacaaggccgtagtcagcttatcaaatggagttagtgtcttaaccagcaaagtgttagacctcaaaaactatatagataaacaattgttacctattgtgaataagcaaagctgcagaatatcaaatatagaaactgtgatagagttccaacaaaagaacaacagactactagagattaccagggaatttagtgttaatgcaggtgtaactacacctgtaagcacttacatgttaactaatagtgaattattgtcattaatcaatgatatgcctataacaaatgatcagaaaaagttaatgtccaacaatgttcaaatagttagacagcaaagttactctatcatgtccataataaaagaggaagtcttagcatatgtagtacaattaccactatatggtgtgatagatacaccttgttggaaattacacacatcccctctatgtacaaccaacacaaaagaagggtcaaacatctgtttaacaagaactgacagaggatggtactgtgacaatgcaggatcagtatctttcttcccacaagctgaaacatgtaaagttcaatcgaatcgagtattttgtgacacaatgaacagtttaacattaccaagtgaagtaaatctctgcaatgttgacatattcaatcccaaatatgattgtaaaattatgacttcaaaaacagatgtaagcagctccgttatcacatctctaggagccattgtgtcatgctatggcaaaactaaatgtacagcatccaataaaaatcgtggaatcataaagacattttctaacgggtgtgattatgtatcaaataaaggggtggacactgtgtctgtaggtaacacattatattatgtaaataagcaagaaggcaaaagtctctatgtaaaaggtgaaccaataataaatttctatgacccattagtattcccctctgatgaatttgatgcatcaatatctcaagtcaatgagaagattaaccagagtttagcatttattcgtaaatccgatgaattattacataatgtaaatgctggtaaatcaaccacaaattga

SEQ ID NO: 14 - protein, 603 aa, Artificial Sequence
  Recombinant F2GF1-5 Chimeric Polypeptide
MGHHHHHHHHHHSSGHIDDDDKHMLESSQNITEEFYQSTCSAVSKGYLSALRTGWYTSVITIELSNIKENKCNGTDAKVKLMKQELDKYKNAVTELQLLMQSTPAANNRARRELPRFMNYTLNNTKKTNVTLSGGPTTVKTKNTTTTQTQPSKPTTKQRQNKPPNKPNNDFHFEVFNFVPCSICSNNPTCWAICKRIPNKKPGKKTTTKPTKKPTFKTTKKDLKPQTTKPKEVPTTKGGEGEVNKIKSALLSTNKAVVSLSNGVSVLTSKVLDLKNYIDKQLLPIVNKQSCRISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTN

SEQ ID NO: 15 - DNA, 1680 bp, Artificial Sequence
  Recombinant F2GF1-6 Chimeric Polynucleotide
atgggccatcatcatcatcatcatcatcatcatcacagcagcggccatatcgacgacgacgacaagcatatgctcgagagtagccaaaacatcactgaagaattttatcaatcaacatgcagtgcagttagcaaaggctatcttagtgctctaagaactggttggtatactagtgttataactatagaattaagtaatatcaaggaaaataagtgtaatggaacagatgctaaggtaaaattgatgaaacaagaattagataaatataaaaatgctgtaacagaattgcagttgctcatgcaaagcacaccagcagcaaacaatcgagccggcggtaaacaacgccaaaacaaaccaccaaacaaacccaataatgattttcacttcgaagtgtttaactttgtaccctgcagcatctgcagcaacaatccaacctgctgggctatctgcaaaagaataccaaacaaaaaaccaggaaagaaaaccaccaccaagcctacaaaaaaaccaaccttcaagacaaccaaaaaagatctcaaacctcaaaccactaaaccaaaggaagtacccaccaccaagggtggcgaaggagaagtgaacaagatcaaaagtgctctactatccacaaacaaggccgtagtcagcttatcaaatggagttagtgtcttaaccagcaaagtgttagacctcaaaaactatatagataaacaattgttacctattgtgaataagcaaagctgcagaatatcaaatatagaaactgtgatagagttccaacaaaagaacaacagactactagagattaccagggaatttagtgttaatgcaggtgtaactacacctgtaagcacttacatgttaactaatagtgaattattgtcattaatcaatgatatgcctataacaaatgatcagaaaaagttaatgtccaacaatgttcaaatagttagacagcaaagttactctatcatgtccataataaaagaggaagtcttagcatatgtagtacaattaccactatatggtgtgatagatacaccttgttggaaattacacacatcccctctatgtacaaccaacacaaaagaagggtcaaacatctgtttaacaagaactgacagaggatggtactgtgacaatgcaggatcagtatctttcttcccacaagctgaaacatgtaaagttcaatcgaatcgagtattttgtgacacaatgaacagtttaacattaccaagtgaagtaaatctctgcaatgttgacatattcaatcccaaatatgattgtaaaattatgacttcaaaaacagatgtaagcagctccgttatcacatctctaggagccattgtgtcatgctatggcaaaactaaatgtacagcatccaataaaaatcgtggaatcataaagacattttctaacgggtgtgattatgtatcaaataaaggggtggacactgtgtctgtaggtaacacattatattatgtaaataagcaagaaggcaaaagtctctatgtaaaaggtgaaccaataataaatttctatgacccattagtattcccctctgatgaatttgatgcatcaatatctcaagtcaatgagaagattaaccagagtttagcatttattcgtaaatccgatgaattattacataatgtaaatgctggtaaatcaaccacaaattga

SEQ ID NO: 16 - protein, 559 aa, Artificial Sequence
  Recombinant F2GF1-6 - Chimeric Polypeptide
MGHHHHHHHHHHSSGHIDDDDKHMLESSQNITEEFYQSTCSAVSKGYLSALRTGWYTSVITIELSNIKENKCNGTDAKVKLMKQELDKYKNAVTELQLLMQSTPAANNRAGGKQRQNKPPNKPNNDFHFEVFNFVPCSICSNNPTCWAICKRIPNKKPGKKTTTKPTKKPTFKTTKKDLKPQTTKPKEVPTTKGGEGEVNKIKSALLSTNKAVVSLSNGVSVLTSKVLDLKNYIDKQLLPIVNKQSCRISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTN

SEQ ID NO: 17 - DNA, 1743 bp, Artificial Sequence
  Recombinant F2GF1-7 Chimeric Polynucleotide
atgggccatcatcatcatcatcatcatcatcatcacagcagcggccatatcgacgacgacgacaagcatatgctcgagagtagccaaaacatcactgaagaattttatcaatcaacatgcagtgcagttagcaaaggctatcttagtgctctaagaactggttggtatactagtgttataactatagaattaagtaatatcaaggaaaataagtgtaatggaacagatgctaaggtaaaattgataaaacaagaattagataaatataaaaatgctgtaacagaattgcagttgctcatgcaaagcacaccagcagcaaacaatcgagccggcggtcccacaacagtcaagactaaaaacacaacaacaacccaaacacaacccagcaagcccactacaaaacaacgccaaaacaaaccaccaaacaaacccaataatgattttcacttcgaagtgtttaactttgtaccctgcagcatctgcagcaacaatccaacctgctgggctatctgcaaaagaataccaaacaaaaaaccaggaaagaaaaccaccaccaagcctacaaaaaaaccaaccttcaagacaaccaaaaaagatctcaaacctcaaaccactaaaccaaaggaagtacccaccaccaagggtggcgaaggagaagtgaacaagatcaaaagtgctctactatccacaaacaaggccgtagtcagcttatcaaatggagttagtgtcttaaccagcaaagtgttagacctcaaaaactatatagataaacaattgttacctattgtgaataagcaaagctgcagaatatcaaatatagaaactgtgatagagttccaacaaaagaacaacagactactagagattaccagggaatttagtgttaatgcaggtgtaactacacctgtaagcacttacatgttaactaatagtgaattattgtcattaatcaatgatatgcctataacaaatgatcagaaaaagttaatgtccaacaatgttcaaatagttagacagcaaagttactctatcatgtccataataaaagaggaagtcttagcatatgtagtacaattaccactatatggtgtgatagatacaccttgttggaaattacacacatcccctctatgtacaaccaacacaaaagaagggtcaaacatctgtttaacaagaactgacagaggatggtactgtgacaatgcaggatcagtatctttcttcccacaagctgaaacatgtaaagttcaatcgaatcgagtattttgtgacacaatgaacagtttaacattaccaagtgaagtaaatctctgcaatgttgacatattcaatcccaaatatgattgtaaaattatgacttcaaaaacagatgtaagcagctccgttatcacatctctaggagccattgtgtcatgctatggcaaaactaaatgtacagcatccaataaaaatcgtggaatcataaagacattttctaacgggtgtgattatgtatcaaataaaggggtggacactgtgtctgtaggtaacacattatattatgtaaataagcaagaaggcaaaagtctctatgtaaaaggtgaaccaataataaatttctatgacccattagtattcccctctgatgaatttgatgcatcaatatctcaagtcaatgagaagattaaccagagtttagcatttattcgtaaatccgatgaattattacataatgtaaatgctggtaaatcaaccacaaattga

SEQ ID NO: 18 - protein, 580 aa, Artificial Sequence
  Recombinant F2GF1-7 Chimeric Polypeptide
MGHHHHHHHHHHSSGHIDDDDKHMLESSQNITEEFYQSTCSAVSKGYLSALRTGWYTSVITIELSNIKENKCNGTDAKVKLIKQELDKYKNAVTELQLLMQSTPAANNRAGGPTTVKTKNTTTTQTQPSKPTTKQRQNKPPNKPNNDFHFEVFNFVPCSICSNNPTCWAICKRIPNKKPGKKTTTKPTKKPTFKTTKKDLKPQTTKPKEVPTTKGGEGEVNKIKSALLSTNKAVVSLSNGVSVLTSKVLDLKNYIDKQLLPIVNKQSCRISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTN

SEQ ID NO: 19 - DNA, 1749 bp, Artificial Sequence
  Recombinant F2GF1-8 Chimeric Polynucleotide
atgggccatcatcatcatcatcatcatcatcatcacagcagcggccatatcgacgacgacgacaagcatatgctcgagagtagccaaaacatcactgaagaattttatcaatcaacatgcagtgcagttagcaaaggctatcttagtgctctaagaactggttggtatactagtgttataactatagaattaagtaatatcaaggaaaataagtgtaatggaacagatgctaaggtaaaattgatgaaacaagaattagataaatataaaaatgctgtaacagaattgcagttgctcatgcaaagcacaccagcagcaaacaatcgagccagaagagaactaccaaggtttatgaattatacactcaacaataccaaaaaaaccaatgtaacattaagcggcggtaaacaacgccaaaacaaaccaccaaacaaacccaataatgattttcacttcgaagtgtttaactttgtaccctgcagcatctgcagcaacaatccaacctgctgggctatctgcaaaagaataccaaacaaaaaaccaggaaagaaaaccaccaccaagcctacaaaaaaaccaaccttcaagacaaccaaaaaagatctcaaacctcaaaccactaaaccaaaggaagtacccaccaccaagggtggcgaaggagaagtgaacaagatcaaaagtgctctactatccacaaacaaggccgtagtcagcttatcaaatggagttagtgtcttaaccagcaaagtgttagacctcaaaaactatatagataaacaattgttacctattgtgaataagcaaagctgcagaatatcaaatatagaaactgtgatagagttccaacaaaagaacaacagactactagagattaccagggaatttagtgttaatgcaggtgtaactacacctgtaagcacttacatgttaactaatagtgaattattgtcattaatcaatgatatgcctataacaaatgatcagaaaaagttaatgtccaacaatgttcaaatagttagacagcaaagttactctatcatgtccataataaaagaggaagtcttagcatatgtagtacaattaccactatatggtgtgatagatacaccttgttggaaattacacacatcccctctatgtacaaccaacacaaaagaagggtcaaacatctgtttaacaagaactgacagaggatggtactgtgacaatgcaggatcagtatctttcttcccacaagctgaaacatgtaaagttcaatcgaatcgagtattttgtgacacaatgaacagtttaacattaccaagtgaagtaaatctctgcaatgttgacatattcaatcccaaatatgattgtaaaattatgacttcaaaaacagatgtaagcagctccgttatcacatctctaggagccattgtgtcatgctatggcaaaactaaatgtacagcatccaataaaaatcgtggaatcataaagacattttctaacgggtgtgattatgtatcaaataaaggggtggacactgtgtctgtaggtaacacattatattatgtaaataagcaagaaggcaaaagtctctatgtaaaaggtgaaccaataataaatttctatgacccattagtattcccctctgatgaatttgatgcatcaatatctcaagtcaatgagaagattaaccagagtttagcatttattcgtaaatccgatgaattattacataatgtaaatgctggtaaatcaaccacaaattga

SEQ ID NO: 20 - protein, 582 aa, Artificial Sequence
  Recombinant F2GF1-8 - Chimeric Polypeptide
MGHHHHHHHHHHSSGHIDDDDKHMLESSQNITEEFYQSTCSAVSKGYLSALRTGWYTSVITIELSNIKENKCNGTDAKVKLMKQELDKYKNAVTELQLLMQSTPAANNRARRELPRFMNYTLNNTKKTNVTLSGGKQRQNKPPNKPNNDFHFEVFNFVPCSICSNNPTCWAICKRIPNKKPGKKTTTKPTKKPTFKTTKKDLKPQTTKPKEVPTTKGGEGEVNKIKSALLSTNKAVVSLSNGVSVLTSKVLDLKNYIDKQLLPIVNKQSCRISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTN

SEQ ID NO: 21 - DNA, 1812 bp, Artificial Sequence
  Recombinant F2GF1-1 C-V1 Chimeric Polynucleotide
atgggccatcatcatcatcatcatcatcatcaccatagcagcggccatatcgacgacgacgacaagcatatgctcgagagtagccaaaacatcactgaagaattttatcaatcaacatgcagtgcagttagcaaaggctatcttagtgctctaagaactggttggtatactagtgttataactatagaattaagtaatatcaaggaaaataagtgtaatggaacagatgctaaggtaaaattgataaaacaagaattagataaatataaaaatgctgtaacagaattgcagttgctcatgcaaagcacaccagcagcaaacaatcgagccagaagagaactaccaaggtttatgaattatacactcaacaataccaaaaaaaccaatgtaacattaagcggcggtcccacaacagtcaagactaaaaacacaacaacaacccaaacacaacccagcaagcccactacaaaacaacgccaaaacaaaccaccaaacaaacccaataatgattttcacttcgaagtgtttaactttgtaccctgcagcatctgcagcaacaatccaacctgctgggctatctgcaaaagaataccagctaaaaaaccaggaaagaaaaccaccaccaagcctacaaaaaaaccaaccttcaagacaaccaaaaaagatctcaaacctcaaaccactaaaccaaaggaagtacccaccaccaagggtggcgaaggagaagtgaacaagatcaaaagtgctctactatccacaaacaaggccgtagtcagcttatcaaatggagttagtgtcttaaccagcaaagtgttagacctcaaaaactatatagataaacaattgttacctattgtgaataagcaaagctgcagaatatcaaatatagaaactgtgatagagttccaacaaaagaacaacagactactagagattaccagggaatttagtgttaatgcaggtgtaactacacctgtaagcacttacatgttaactaatagtgaattattgtcattaatcaatgatatgcctataacaaatgatcagaaaaagttaatgtccaacaatgttcaaatagttagacagcaaagttactctatcatgtccataataaaagaggaagtcttagcatatgtagtacaattaccactatatggtgtgatagatacaccttcttggaaattacacacatcccctctatgtacaaccaacacaaaagaagggtcaaacatctctttaacaagaactgacagaggatggtactctgacaatgcaggatcagtatctttcttcccacaagctgaaacatgtaaagttcaatcgaatcgagtattttgtgacacaatgaacagtttaacattaccaagtgaagtaaatctctgcaatgttgacatattcaatcccaaatatgattgtaaaattatgacttcaaaaacagatgtaagcagctccgttatcacatctctaggagccattgtgtcatgctatggcaaaactaaatgtacagcatccaataaaaatcgtggaatcataaagacattttctaacgggtctgattatgtatcaaataaaggggtggacactgtgtctgtaggtaacacattatattatgtaaataagcaagaaggcaaaagtctctatgtaaaaggtgaaccaataataaatttctatgacccattagtattcccctctgatgaatttgatgcatcaatatctcaagtcaatgagaagattaaccagagtttagcatttattcgtaaatccgatgaattattacataatgtaaatgctggtaaatcaaccacaaattga

SEQ ID NO: 22 - DNA, 1812 bp, Artificial Sequence
  Recombinant F2GF1-1 C-V2 Chimeric Polynucleotide
atgggccatcatcatcatcatcatcatcatcatcacagcagcggccatatcgacgacgacgacaagcatatgctcgagagtagccaaaacatcactgaagaattttatcaatcaacatccagtgcagttagcaaaggctatcttagtgctctaagaactggttggtatactagtgttataactatagaattaagtaatatcaaggaaaataagtctaatggaacagatgctaaggtaaaattgataaaacaagaattagataaatataaaaatgctgtaacagaattgcagttgctcatgcaaagcacaccagcagcaaacaatcgagccagaagagaactaccaaggtttatgaattatacactcaacaataccaaaaaaaccaatgtaacattaagcggcggtcccacaacagtcaagactaaaaacacaacaacaacccaaacacaacccagcaagcccactacaaaacaacgccaaaacaaaccaccaaacaaacccaataatgattttcacttcgaagtgtttaactttgtaccctgcagcatctgcagcaacaatccaacctgctgggctatctgcaaaagaataccagctaaaaaaccaggaaagaaaaccaccaccaagcctacaaaaaaaccaaccttcaagacaaccaaaaaagatctcaaacctcaaaccactaaaccaaaggaagtacccaccaccaagggtggcgaaggagaagtgaacaagatcaaaagtgctctactatccacaaacaaggccgtagtcagcttatcaaatggagttagtgtcttaaccagcaaagtgttagacctcaaaaactatatagataaacaattgttacctattgtgaataagcaaagctccagaatatcaaatatagaaactgtgatagagttccaacaaaagaacaacagactactagagattaccagggaatttagtgttaatgcaggtgtaactacacctgtaagcacttacatgttaactaatagtgaattattgtcattaatcaatgatatgcctataacaaatgatcagaaaaagttaatgtccaacaatgttcaaatagttagacagcaaagttactctatcatgtccataataaaagaggaagtcttagcatatgtagtacaattaccactatatggtgtgatagatacaccttgttggaaattacacacatcccctctatctacaaccaacacaaaagaagggtcaaacatctgtttaacaagaactgacagaggatggtactgtgacaatgcaggatcagtatctttcttcccacaagctgaaacatgtaaagttcaatcgaatcgagtattttgtgacacaatgaacagtttaacattaccaagtgaagtaaatctctgcaatgttgacatattcaatcccaaatatgattgtaaaattatgacttcaaaaacagatgtaagcagctccgttatcacatctctaggagccattgtgtcatgctatggcaaaactaaatgtacagcatccaataaaaatcgtggaatcataaagacattttctaacgggtgtgattatgtatcaaataaaggggtggacactgtgtctgtaggtaacacattatattatgtaaataagcaagaaggcaaaagtctctatgtaaaaggtgaaccaataataaatttctatgacccattagtattcccctctgatgaatttgatgcatcaatatctcaagtcaatgagaagattaaccagagtttagcatttattcgtaaatccgatgaattattacataatgtaaatgctggtaaatcaaccacaaattga

SEQ ID NO: 23 - DNA, 1812 bp, Artificial Sequence
  Recombinant F2GF1-1 C-V12 Chimeric Polynucleotide
atgggccatcatcatcatcatcatcatcatcatcacagcagcggccatatcgacgacgacgacaagcatatgctcgagagtagccaaaacatcactgaagaattttatcaatcaacatccagtgcagttagcaaaggctatcttagtgctctaagaactggttggtatactagtgttataactatagaattaagtaatatcaaggaaaataagtctaatggaacagatgctaaggtaaaattgataaaacaagaattagataaatataaaaatgctgtaacagaattgcagttgctcatgcaaagcacaccagcagcaaacaatcgagccagaagagaactaccaaggtttatgaattatacactcaacaataccaaaaaaaccaatgtaacattaagcggcggtcccacaacagtcaagactaaaaacacaacaacaacccaaacacaacccagcaagcccactacaaaacaacgccaaaacaaaccaccaaacaaacccaataatgattttcacttcgaagtgtttaactttgtaccctgcagcatctgcagcaacaatccaacctgctgggctatctgcaaaagaataccagctaaaaaaccaggaaagaaaaccaccaccaagcctacaaaaaaaccaaccttcaagacaaccaaaaaagatctcaaacctcaaaccactaaaccaaaggaagtacccaccaccaagggtggcgaaggagaagtgaacaagatcaaaagtgctctactatccacaaacaaggccgtagtcagcttatcaaatggagttagtgtcttaaccagcaaagtgttagacctcaaaaactatatagataaacaattgttacctattgtgaataagcaaagctccagaatatcaaatatagaaactgtgatagagttccaacaaaagaacaacagactactagagattaccagggaatttagtgttaatgcaggtgtaactacacctgtaagcacttacatgttaactaatagtgaattattgtcattaatcaatgatatgcctataacaaatgatcagaaaaagttaatgtccaacaatgttcaaatagttagacagcaaagttactctatcatgtccataataaaagaggaagtcttagcatatgtagtacaattaccactatatggtgtgatagatacaccttcttggaaattacacacatcccctctatctacaaccaacacaaaagaagggtcaaacatctctttaacaagaactgacagaggatggtactctgacaatgcaggatcagtatctttcttcccacaagctgaaacatgtaaagttcaatcgaatcgagtattttgtgacacaatgaacagtttaacattaccaagtgaagtaaatctctgcaatgttgacatattcaatcccaaatatgattgtaaaattatgacttcaaaaacagatgtaagcagctccgttatcacatctctaggagccattgtgtcatgctatggcaaaactaaatgtacagcatccaataaaaatcgtggaatcataaagacattttctaacgggtctgattatgtatcaaataaaggggtggacactgtgtctgtaggtaacacattatattatgtaaataagcaagaaggcaaaagtctctatgtaaaaggtgaaccaataataaatttctatgacccattagtattcccctctgatgaatttgatgcatcaatatctcaagtcaatgagaagattaaccagagtttagcatttattcgtaaatccgatgaattattacataatgtaaatgctggtaaatcaaccacaaattga

SEQ ID NO: 24 - DNA, 1812 bp, Artificial Sequence; identical to SEQ ID NO: 22
  Recombinant F2GF1-1 C-V12′ Chimeric Polynucleotide

SEQ ID NO: 25 - DNA, 1671 bp, Artificial Sequence
  Recombinant F2GF1-1 de11 Chimeric Polynucleotide
atgggccatcatcatcatcatcatcatcatcatcacagcagcggccatatcgacgacgacgacaagcatatgctcgagagtagccaaaacatcactgaagaattttatcaatcaacatgcagtgcagttagcaaaggctatcttagtgctctaagaactggttggtatactagtgttataactatagaattaagtaatatcaaggaaaataagtgtaatggaacagatgctaaggtaaaattgataaaacaagaattagataaatataaaaatgctgtaacagaattgcagttgctcatgcaaagcacaccagcagcaaacaatcgagccagaagagaactaccaaggtttatgaattatacactcaacaataccaaaaaaaccaatgtaacattaagcggcggtcccacaacagtcaagactaaaaacacaacaacaacccaaacacaacccagcaagcccactacaaaacaacgccaaaacaaaccaccaaacaaacccaataatgattttcacttcgaagtgtttaactttgtaccctgcagcatctgcagcaacaatccaacctgctgggctatctgcaaaagaataccagctaaaaaaccaggaaagaaaaccaccaccaagcctacaaaaaaaccaaccttcaagacaaccaaaaaagatctcaaacctcaaaccactaaaccaaaggaagtacccaccaccaaggaattcaataagcaaagctgcagaatatcaaatatagaaactgtgatagagttccaacaaaagaacaacagactactagagattaccagggaatttagtgttaatgcaggtgtaactacacctgtaagcacttacatgttaactaatagtgaattattgtcattaatcaatgatatgcctataacaaatgatcagaaaaagttaatgtccaacaatgttcaaatagttagacagcaaagttactctatcatgtccataataaaagaggaagtcttagcatatgtagtacaattaccactatatggtgtgatagatacaccttgttggaaattacacacatcccctctatgtacaaccaacacaaaagaagggtcaaacatctgtttaacaagaactgacagaggatggtactgtgacaatgcaggatcagtatctttcttcccacaagctgaaacatgtaaagttcaatcgaatcgagtattttgtgacacaatgaacagtttaacattaccaagtgaagtaaatctctgcaatgttgacatattcaatcccaaatatgattgtaaaattatgacttcaaaaacagatgtaagcagctccgttatcacatctctaggagccattgtgtcatgctatggcaaaactaaatgtacagcatccaataaaaatcgtggaatcataaagacattttctaacgggtgtgattatgtatcaaataaaggggtggacactgtgtctgtaggtaacacattatattatgtaaataagcaagaaggcaaaagtctctatgtaaaaggtgaaccaataataaatttctatgacccattagtattcccctctgatgaatttgatgcatcaatatctcaagtcaatgagaagattaaccagagtttagcatttattcgtaaatccgatgaattattacataatgtaaatgctggtaaatcaaccacaaattga

SEQ ID NO: 26 - DNA, 1686 bp, Artificial Sequence
  Recombinant F2GF1-1 de12 Chimeric Polynucleotide
atgggccatcatcatcatcatcatcatcatcatcacagcagcggccatatcgacgacgacgacaagcatatgctcgagagtagccaaaacatcactgaagaattttatcaatcaacatgcagtgcagttagcaaaggctatcttagtgctctaagaactggttggtatactagtgttataactatagaattaagtaatatcaaggaaaataagtgtaatggaacagatgctaaggtaaaattgataaaacaagaattagataaatataaaaatgctgtaacagaattgcagttgctcatgcaaagcacaccagcagcaaacaatcgagccagaagagaactaccaaggtttatgaattatacactcaacaataccaaaaaaaccaatgtaacattaagcggcggtcccacaacagtcaagactaaaaacacaacaacaacccaaacacaacccagcaagcccactacaaaacaacgccaaaacaaaccaccaaacaaacccaataatgattttcacttcgaagtgtttaactttgtaccctgcagcatctgcagcaacaatccaacctgctgggctatctgcaaaagaataccagctaaaaaaccaggaaagaaaaccaccaccaagcctacaaaaaaaccaaccttcaagacaaccaaaaaagatctcaaacctcaaaccactaaaccaaaggaagtacccaccaccaaggaattcttgttacctattgtgaataagcaaagctgcagaatatcaaatatagaaactgtgatagagttccaacaaaagaacaacagactactagagattaccagggaatttagtgttaatgcaggtgtaactacacctgtaagcacttacatgttaactaatagtgaattattgtcattaatcaatgatatgcctataacaaatgatcagaaaaagttaatgtccaacaatgttcaaatagttagacagcaaagttactctatcatgtccataataaaagaggaagtcttagcatatgtagtacaattaccactatatggtgtgatagatacaccttgttggaaattacacacatcccctctatgtacaaccaacacaaaagaagggtcaaacatctgtttaacaagaactgacagaggatggtactgtgacaatgcaggatcagtatctttcttcccacaagctgaaacatgtaaagttcaatcgaatcgagtattttgtgacacaatgaacagtttaacattaccaagtgaagtaaatctctgcaatgttgacatattcaatcccaaatatgattgtaaaattatgacttcaaaaacagatgtaagcagctccgttatcacatctctaggagccattgtgtcatgctatggcaaaactaaatgtacagcatccaataaaaatcgtggaatcataaagacattttctaacgggtgtgattatgtatcaaataaaggggtggacactgtgtctgtaggtaacacattatattatgtaaataagcaagaaggcaaaagtctctatgtaaaaggtgaaccaataataaatttctatgacccattagtattcccctctgatgaatttgatgcatcaatatctcaagtcaatgagaagattaaccagagtttagcatttattcgtaaatccgatgaattattacataatgtaaatgctggtaaatcaaccacaaattga

SEQ ID NO: 27 - DNA, 1740 bp, Artificial Sequence
  Recombinant F2GF1-1 de13 Chimeric Polynucleotide
atgggccatcatcatcatcatcatcatcatcatcacagcagcggccatatcgacgacgacgacaagcatatgctcgagagtagccaaaacatcactgaagaattttatcaatcaacatgcagtgcagttagcaaaggctatcttagtgctctaagaactggttggtatactagtgttataactatagaattaagtaatatcaaggaaaataagtgtaatggaacagatgctaaggtaaaattgataaaacaagaattagataaatataaaaatgctgtaacagaattgcagttgctcatgcaaagcacaccagcagcaaacaatcgagccagaagagaactaccaaggtttatgaattatacactcaacaataccaaaaaaaccaatgtaacattaagcggcggtcccacaacagtcaagactaaaaacacaacaacaacccaaacacaacccagcaagcccactacaaaacaacgccaaaacaaaccaccaaacaaacccaataatgattttcacttcgaagtgtttaactttgtaccctgcagcatctgcagcaacaatccaacctgctgggctatctgcaaaagaataccagctaaaaaaccaggaaagaaaaccaccaccaagcctacaaaaaaaccaaccttcaagacaaccaaaaaagatctcaaacctcaaaccactaaaccaaaggaagtacccaccaccaaggaattcgttagtgtcttaaccagcaaagtgttagacctcaaaaactatatagataaacaattgttacctattgtgaataagcaaagctgcagaatatcaaatatagaaactgtgatagagttccaacaaaagaacaacagactactagagattaccagggaatttagtgttaatgcaggtgtaactacacctgtaagcacttacatgttaactaatagtgaattattgtcattaatcaatgatatgcctataacaaatgatcagaaaaagttaatgtccaacaatgttcaaatagttagacagcaaagttactctatcatgtccataataaaagaggaagtcttagcatatgtagtacaattaccactatatggtgtgatagatacaccttgttggaaattacacacatcccctctatgtacaaccaacacaaaagaagggtcaaacatctgtttaacaagaactgacagaggatggtactgtgacaatgcaggatcagtatctttcttcccacaagctgaaacatgtaaagttcaatcgaatcgagtattttgtgacacaatgaacagtttaacattaccaagtgaagtaaatctctgcaatgttgacatattcaatcccaaatatgattgtaaaattatgacttcaaaaacagatgtaagcagctccgttatcacatctctaggagccattgtgtcatgctatggcaaaactaaatgtacagcatccaataaaaatcgtggaatcataaagacattttctaacgggtgtgattatgtatcaaataaaggggtggacactgtgtctgtaggtaacacattatattatgtaaataagcaagaaggcaaaagtctctatgtaaaaggtgaaccaataataaatttctatgacccattagtattcccctctgatgaatttgatgcatcaatatctcaagtcaatgagaagattaaccagagtttagcatttattcgtaaatccgatgaattattacataatgtaaatgctggtaaatcaaccacaaattga

SEQ ID NO: 28 - DNA, 1680 bp, Artificial Sequence
  Recombinant F2GF1-1 de14 Chimeric Polynucleotide
atgggccatcatcatcatcatcatcatcatcatcacagcagcggccatatcgacgacgacgacaagcatatgctcgagagtagccaaaacatcactgaagaattttatcaatcaacatgcagtgcagttagcaaaggctatcttagtgctctaagaactggttggtatactagtgttataactatagaattaagtaatatcaaggaaaataagtgtaatggaacagatgctaaggtaaaattgataaaacaagaattagataaatataaaaatgctgtaacagaattgcagttgctcatgcaaagcacaccagcagcaaacaatcgagccagaagagaactaccaaggtttatgaattatacactcaacaataccaaaaaaaccaatgtaacattaagcggcggtcccacaacagtcaagactaaaaacacaacaacaacccaaacacaacccagcaagcccactacaaaacaacgccaaaacaaaccaccaaacaaacccaataatgattttcacttcgaagtgtttaactttgtaccctgcagcatctgcagcaacaatccaacctgctgggctatctgcaaaagaataccagctaaaaaaccaggaaagaaaaccaccaccaagcctacaaaaaaaccaaccttcaagacaaccaaaaaagatctcaaacctcaaaccactaaaccaaaggaagtacccaccaccaaggaattccctattgtgaataagcaaagctgcagaatatcaaatatagaaactgtgatagagttccaacaaaagaacaacagactactagagattaccagggaatttagtgttaatgcaggtgtaactacacctgtaagcacttacatgttaactaatagtgaattattgtcattaatcaatgatatgcctataacaaatgatcagaaaaagttaatgtccaacaatgttcaaatagttagacagcaaagttactctatcatgtccataataaaagaggaagtcttagcatatgtagtacaattaccactatatggtgtgatagatacaccttgttggaaattacacacatcccctctatgtacaaccaacacaaaagaagggtcaaacatctgtttaacaagaactgacagaggatggtactgtgacaatgcaggatcagtatctttcttcccacaagctgaaacatgtaaagttcaatcgaatcgagtattttgtgacacaatgaacagtttaacattaccaagtgaagtaaatctctgcaatgttgacatattcaatcccaaatatgattgtaaaattatgacttcaaaaacagatgtaagcagctccgttatcacatctctaggagccattgtgtcatgctatggcaaaactaaatgtacagcatccaataaaaatcgtggaatcataaagacattttctaacgggtgtgattatgtatcaaataaaggggtggacactgtgtctgtaggtaacacattatattatgtaaataagcaagaaggcaaaagtctctatgtaaaaggtgaaccaataataaatttctatgacccattagtattcccctctgatgaatttgatgcatcaatatctcaagtcaatgagaagattaaccagagtttagcatttattcgtaaatccgatgaattattacataatgtaaatgctggtaaatcaaccacaaattga

SEQ ID NO: 29 - DNA, 1548 bp, Artificial Sequence
  Recombinant F2GF1-1 de15 Chimeric Polynucleotide
atgggccatcatcatcatcatcatcatcatcatcacagcagcggccatatcgacgacgacgacaagcatatgctcgagagtagccaaaacatcactgaagaattttatcaatcaacatgcagtgcagttagcaaaggctatcttagtgctctaagaactggttggtatactagtgttataactatagaattaagtaatatcaaggaaaataagtgtaatggaacagatgctaaggtaaaattgataaaacaagaattagataaatataaaaatgctgtaacagaattgcagttgctcatgcaaagcacaccagcagcaaacaatcgagccagaagagaactaccaaggtttatgaattatacactcaacaataccaaaaaaaccaatgtaacattaagcggcggtcccacaacagtcaagactaaaaacacaacaacaacccaaacacaacccagcaagcccactacaaaacaacgccaaaacaaaccaccaaacaaacccaataatgattttcacttcgaagtgtttaactttgtaccctgcagcatctgcagcaacaatccaacctgctgggctatctgcaaaagaataccagctaaaaaaccaggaaagaaaaccaccaccaagcctacaaaaaaaccaaccttcaagacaaccaaaaaagatctcaaacctcaaaccactaaaccaaaggaagtacccaccaccaaggaattcattgtgaataagcaaagctgcagaatatcaaatatagaaactgtgatagagttccaacaaaagaacaacagactactagagattaccagggaatttagtgttaatgcaggtgtaactacacctgtaagcacttacatgttaactaatagtgaattattgtcattaatcaatgatatgcctataacaaatgatcagaaaaagttaatgtccaacaatgttcaaatagttagacagcaaagttactctatcatgtccataataaaagaggaagtcttagcatatgtagtacaattaccactatatggtgtgatagatacaccttgttggaaattacacacatcccctctatgtacaaccaacacaaaagaagggtcaaacatctgtttaacaagaactgacagaggatggtactgtgacaatgcaggatcagtatctttcttcccacaagctgaaacatgtaaagttcaatcgaatcgagtattttgtgacacaatgaacagtttaacattaccaagtgaagtaaatctctgcaatgttgacatattcaatcccaaatatgattgtaaaattatgacttcaaaaacagatgtaagcagctccgttatcacatctctaggagccattgtgtcatgctatggcaaaactaaatgtacagcatccaataaaaatcgtggaatcataaagacattttctaacgggtgtgattatgtatcaaataaaggggtggacactgtgtctgtaggtaacacattatattatgtaaataagcaagaaggcaaaagtctctatgtaaaaggtgaaccaataataaatttctatgacccattatga

SEQ ID NO: 30 - DNA, 1572 bp, Artificial Sequence
  Recombinant F2GF1-1 de16 Chimeric Polynucleotide
atgggccatcatcatcatcatcatcatcatcatcacagcagcggccatatcgacgacgacgacaagcatatgctcgagagtagccaaaacatcactgaagaattttatcaatcaacatgcagtgcagttagcaaaggctatcttagtgctctaagaactggttggtatactagtgttataactatagaattaagtaatatcaaggaaaataagtgtaatggaacagatgctaaggtaaaattgataaaacaagaattagataaatataaaaatgctgtaacagaattgcagttgctcatgcaaagcacaccagcagcaaacaatcgagccagaagagaactaccaaggtttatgaattatacactcaacaataccaaaaaaaccaatgtaacattaagcggcggtcccacaacagtcaagactaaaaacacaacaacaacccaaacacaacccagcaagcccactacaaaacaacgccaaaacaaaccaccaaacaaacccaataatgattttcacttcgaagtgtttaactttgtaccctgcagcatctgcagcaacaatccaacctgctgggctatctgcaaaagaataccagctaaaaaaccaggaaagaaaaccaccaccaagcctacaaaaaaaccaaccttcaagacaaccaaaaaagatctcaaacctcaaaccactaaaccaaaggaagtacccaccaccaaggaattcgataaacaattgttacctattgtgaataagcaaagctgcagaatatcaaatatagaaactgtgatagagttccaacaaaagaacaacagactactagagattaccagggaatttagtgttaatgcaggtgtaactacacctgtaagcacttacatgttaactaatagtgaattattgtcattaatcaatgatatgcctataacaaatgatcagaaaaagttaatgtccaacaatgttcaaatagttagacagcaaagttactctatcatgtccataataaaagaggaagtcttagcatatgtagtacaattaccactatatggtgtgatagatacaccttgttggaaattacacacatcccctctatgtacaaccaacacaaaagaagggtcaaacatctgtttaacaagaactgacagaggatggtactgtgacaatgcaggatcagtatctttcttcccacaagctgaaacatgtaaagttcaatcgaatcgagtattttgtgacacaatgaacagtttaacattaccaagtgaagtaaatctctgcaatgttgacatattcaatcccaaatatgattgtaaaattatgacttcaaaaacagatgtaagcagctccgttatcacatctctaggagccattgtgtcatgctatggcaaaactaaatgtacagcatccaataaaaatcgtggaatcataaagacattttctaacgggtgtgattatgtatcaaataaaggggtggacactgtgtctgtaggtaacacattatattatgtaaataagcaagaaggcaaaagtctctatgtaaaaggtgaaccaataataaatttctatgacccattagtattctag

SEQ ID NO: 31 - DNA, 1548 bp, Artificial Sequence
  Recombinant F2GF1-1 de15 C-V12 Chimeric Polynucleotide
atgggccatcatcatcatcatcatcatcatcatcacagcagcggccatatcgacgacgacgacaagcatatgctcgagagtagccaaaacatcactgaagaattttatcaatcaacatccagtgcagttagcaaaggctatcttagtgctctaagaactggttggtatactagtgttataactatagaattaagtaatatcaaggaaaataagtctaatggaacagatgctaaggtaaaattgataaaacaagaattagataaatataaaaatgctgtaacagaattgcagttgctcatgcaaagcacaccagcagcaaacaatcgagccagaagagaactaccaaggtttatgaattatacactcaacaataccaaaaaaaccaatgtaacattaagcggcggtcccacaacagtcaagactaaaaacacaacaacaacccaaacacaacccagcaagcccactacaaaacaacgccaaaacaaaccaccaaacaaacccaataatgattttcacttcgaagtgtttaactttgtaccctgcagcatctgcagcaacaatccaacctgctgggctatctgcaaaagaataccagctaaaaaaccaggaaagaaaaccaccaccaagcctacaaaaaaaccaaccttcaagacaaccaaaaaagatctcaaacctcaaaccactaaaccaaaggaagtacccaccaccaaggaattcattgtgaataagcaaagctccagaatatcaaatatagaaactgtgatagagttccaacaaaagaacaacagactactagagattaccagggaatttagtgttaatgcaggtgtaactacacctgtaagcacttacatgttaactaatagtgaattattgtcattaatcaatgatatgcctataacaaatgatcagaaaaagttaatgtccaacaatgttcaaatagttagacagcaaagttactctatcatgtccataataaaagaggaagtcttagcatatgtagtacaattaccactatatggtgtgatagatacaccttcttggaaattacacacatcccctctatctacaaccaacacaaaagaagggtcaaacatctctttaacaagaactgacagaggatggtactctgacaatgcaggatcagtatctttcttcccacaagctgaaacatgtaaagttcaatcgaatcgagtattttgtgacacaatgaacagtttaacattaccaagtgaagtaaatctctgcaatgttgacatattcaatcccaaatatgattgtaaaattatgacttcaaaaacagatgtaagcagctccgttatcacatctctaggagccattgtgtcatgctatggcaaaactaaatgtacagcatccaataaaaatcgtggaatcataaagacattttctaacgggtctgattatgtatcaaataaaggggtggacactgtgtctgtaggtaacacattatattatgtaaataagcaagaaggcaaaagtctctatgtaaaaggtgaaccaataataaatttctatgacccattatga

SEQ ID NO: 32 - DNA, 1572 bp, Artificial Sequence
  Recombinant F2GF1-1 de16 C-V12 Chimeric Polynucleotide
atgggccatcatcatcatcatcatcatcatcatcacagcagcggccatatcgacgacgacgacaagcatatgctcgagagtagccaaaacatcactgaagaattttatcaatcaacatccagtgcagttagcaaaggctatcttagtgctctaagaactggttggtatactagtgttataactatagaattaagtaatatcaaggaaaataagtctaatggaacagatgctaaggtaaaattgataaaacaagaattagataaatataaaaatgctgtaacagaattgcagttgctcatgcaaagcacaccagcagcaaacaatcgagccagaagagaactaccaaggtttatgaattatacactcaacaataccaaaaaaaccaatgtaacattaagcggcggtcccacaacagtcaagactaaaaacacaacaacaacccaaacacaacccagcaagcccactacaaaacaacgccaaaacaaaccaccaaacaaacccaataatgattttcacttcgaagtgtttaactttgtaccctgcagcatctgcagcaacaatccaacctgctgggctatctgcaaaagaataccagctaaaaaaccaggaaagaaaaccaccaccaagcctacaaaaaaaccaaccttcaagacaaccaaaaaagatctcaaacctcaaaccactaaaccaaaggaagtacccaccaccaaggaattcgataaacaattgttacctattgtgaataagcaaagctccagaatatcaaatatagaaactgtgatagagttccaacaaaagaacaacagactactagagattaccagggaatttagtgttaatgcaggtgtaactacacctgtaagcacttacatgttaactaatagtgaattattgtcattaatcaatgatatgcctataacaaatgatcagaaaaagttaatgtccaacaatgttcaaatagttagacagcaaagttactctatcatgtccataataaaagaggaagtcttagcatatgtagtacaattaccactatatggtgtgatagatacaccttcttggaaattacacacatcccctctatctacaaccaacacaaaagaagggtcaaacatctctttaacaagaactgacagaggatggtactctgacaatgcaggatcagtatctttcttcccacaagctgaaacatgtaaagttcaatcgaatcgagtattttgtgacacaatgaacagtttaacattaccaagtgaagtaaatctctgcaatgttgacatattcaatcccaaatatgattgtaaaattatgacttcaaaaacagatgtaagcagctccgttatcacatctctaggagccattgtgtcatgctatggcaaaactaaatgtacagcatccaataaaaatcgtggaatcataaagacattttctaacgggtctgattatgtatcaaataaaggggtggacactgtgtctgtaggtaacacattatattatgtaaataagcaagaaggcaaaagtctctatgtaaaaggtgaaccaataataaatttctatgacccattagtattctag

SEQ ID NO: 33 - DNA, 387 bp, Artificial Sequence
  Recombinant polynucleotide encoding variant RSV G protein
atgggccatcatcatcatcatcatcatcatcatcacagcagcggccatatcgacgacgacgacaagcatatgcccacaacagtcaagactaaaaacacaacaacaacccaaacacaacccagcaagcccactacaaaacaacgccaaaacaaaccaccaaacaaacccaataatgattttcacttcgaagtgtttaactttgtaccctgcagcatctgcagcaacaatccaacctgctgggctatctgcaaaagaataccaaacaaaaaaccaggaaagaaaaccaccaccaagcctacaaaaaaaccaaccttcaagacaaccaaaaaagatctcaaacctcaaaccactaaaccaaaggaagtacccaccaccaagtgaggatcc

SEQ ID NO: 34 - DNA, 387 bp, Artificial Sequence
  Recombinant polunucleotide encoding variant An-G-O RSV G protein
atgggccatcatcatcatcatcatcatcatcatcacagcagcggccatatcgacgacgacgacaagcatatgccgaccactgtgaaaaccaaaaatacgactacgacccaaacgcaaccgagcaaaccgaccactaaacaacgccaaaacaaaccaccgaacaaacccaataatgattttcacttcgaagtgtttaactttgtaccctgcagcatctgcagcaacaatccaacctgctgggctatctgcaaacgtattccaaacaaaaaaccaggaaagaaaaccaccaccaagcctacaaaaaaaccaaccttcaagacaaccaaaaaagatctcaaacctcaaaccactaaaccaaaggaagtacccaccaccaagtgaggatcc

SEQ ID NO: 35 - DNA, 324 bp, Artificial Sequence
  Recombinant polynucleotide encoding variant An-GT RSV G protein
atgggccatcatcatcatcatcatcatcatcatcacagcagcggccatatcgacgacgacgacaagcatatgaaacaacgccaaaacaaaccaccaaacaaacccaataatgattttcacttcgaagtgtttaactttgtaccctgcagcatctgcagcaacaatccaacctgctgggctatctgcaaaagaataccaaacaaaaaaccaggaaagaaaaccaccaccaagcctacaaaaaaaccaaccttcaagacaaccaaaaaagatctcaaacctcaaaccactaaaccaaaggaagtacccaccaccaagtgaggatcc

SEQ ID NO: 36 - DNA, 324 bp, Artificial Sequence
  Recombinant polynucleotide encoding variant An-GT-O RSV G protein
atgggccatcatcatcatcatcatcatcatcatcacagcagcggccatatcgacgacgacgacaagcatatgaaacaacgccaaaacaaaccaccgaacaaacccaataatgattttcacttcgaagtgtttaactttgtaccctgcagcatctgcagcaacaatccaacctgctgggctatctgcaaacgtattccaaacaaaaaaccaggaaagaaaaccaccaccaagcctacaaaaaaaccaaccttcaagacaaccaaaaaagatctcaaacctcaaaccactaaaccaaaggaagtacccaccaccaagtgaggatcc

SEQ ID NO: 37 - DNA, 1161 bp, Artificial Sequence
  Recombinant polynucleotide encoding variant F1-pET19b RSV G protein
atgggccatcatcatcatcatcatcatcatcatcacagcagcggccatatcgacgacgacgacaagcatatgctcgagggagaagtgaacaagatcaaaagtgctctactatccacaaacaaggccgtagtcagcttatcaaatggagttagtgtcttaaccagcaaagtgttagacctcaaaaactatatagataaacaattgttacctattgtgaataagcaaagctgcagaatatcaaatatagaaactgtgatagagttccaacaaaagaacaacagactactagagattaccagggaatttagtgttaatgcaggtgtaactacacctgtaagcacttacatgttaactaatagtgaattattgtcattaatcaatgatatgcctataacaaatgatcagaaaaagttaatgtccaacaatgttcaaatagttagacagcaaagttactctatcatgtccataataaaagaggaagtcttagcatatgtagtacaattaccactatatggtgtgatagatacaccttgttggaaattacacacatcccctctatgtacaaccaacacaaaagaagggtcaaacatctgtttaacaagaactgacagaggatggtactgtgacaatgcaggatcagtatctttcttcccacaagctgaaacatgtaaagttcaatcgaatcgagtattttgtgacacaatgaacagtttaacattaccaagtgaagtaaatctctgcaatgttgacatattcaatcccaaatatgattgtaaaattatgacttcaaaaacagatgtaagcagctccgttatcacatctctaggagccattgtgtcatgctatggcaaaactaaatgtacagcatccaataaaaatcgtggaatcataaagacattttctaacgggtgtgattatgtatcaaataaaggggtggacactgtgtctgtaggtaacacattatattatgtaaataagcaagaaggcaaaagtctctatgtaaaaggtgaaccaataataaatttctatgacccattagtattcccctctgatgaatttgatgcatcaatatctcaagtcaatgagaagattaaccagagtttagcatttattcgtaaatccgatgaattattacataatgtaaatgctggtaaatcatag

SEQ ID NO: 38 - DNA, 903 bp, Artificial Sequence
  Recombinant polynucleotide encoding variant F1 de15 RSV G protein
atgggccatcatcatcatcatcatcatcatcatcacagcagcggccatatcgacgacgacgacaagcatatgctcgagaataagcaaagctgcagaatatcaaatatagaaactgtgatagagttccaacaaaagaacaacagactactagagattaccagggaatttagtgttaatgcaggtgtaactacacctgtaagcacttacatgttaactaatagtgaattattgtcattaatcaatgatatgcctataacaaatgatcagaaaaagttaatgtccaacaatgttcaaatagttagacagcaaagttactctatcatgtccataataaaagaggaagtcttagcatatgtagtacaattaccactatatggtgtgatagatacaccttgttggaaattacacacatcccctctatgtacaaccaacacaaaagaagggtcaaacatctgtttaacaagaactgacagaggatggtactgtgacaatgcaggatcagtatctttcttcccacaagctgaaacatgtaaagttcaatcgaatcgagtattttgtgacacaatgaacagtttaacattaccaagtgaagtaaatctctgcaatgttgacatattcaatcccaaatatgattgtaaaattatgacttcaaaaacagatgtaagcagctccgttatcacatctctaggagccattgtgtcatgctatggcaaaactaaatgtacagcatccaataaaaatcgtggaatcataaagacattttctaacgggtgtgattatgtatcaaataaaggggtggacactgtgtctgtaggtaacacattatattatgtaaataagcaagaaggcaaaagtctctatgtaaaaggtgaaccaataataaatttctatgacccattatag

SEQ ID NO: 39 - DNA, 903 bp, Artificial Sequence
  Recombinant polynucleotide encoding variant  F1 de15 C-V1 RSV G protein
atgggccatcatcatcatcatcatcatcatcatcacagcagcggccatatcgacgacgacgacaagcatatgctcgagaataagcaaagctgcagaatatcaaatatagaaactgtgatagagttccaacaaaagaacaacagactactagagattaccagggaatttagtgttaatgcaggtgtaactacacctgtaagcacttacatgttaactaatagtgaattattgtcattaatcaatgatatgcctataacaaatgatcagaaaaagttaatgtccaacaatgttcaaatagttagacagcaaagttactctatcatgtccataataaaagaggaagtcttagcatatgtagtacaattaccactatatggtgtgatagatacaccttcttggaaattacacacatcccctctatgtacaaccaacacaaaagaagggtcaaacatctctttaacaagaactgacagaggatggtactctgacaatgcaggatcagtatctttcttcccacaagctgaaacatgtaaagttcaatcgaatcgagtattttgtgacacaatgaacagtttaacattaccaagtgaagtaaatctctgcaatgttgacatattcaatcccaaatatgattgtaaaattatgacttcaaaaacagatgtaagcagctccgttatcacatctctaggagccattgtgtcatgctatggcaaaactaaatgtacagcatccaataaaaatcgtggaatcataaagacattttctaacgggtctgattatgtatcaaataaaggggtggacactgtgtctgtaggtaacacattatattatgtaaataagcaagaaggcaaaagtctctatgtaaaaggtgaaccaataataaatttctatgacccattatag

SEQ ID NO: 40 - DNA, 903 bp, Artificial Sequence
  Recombinant polynucleotide encoding variant  F1 de15 c-V2′  RSV G protein
atgggccatcatcatcatcatcatcatcatcatcacagcagcggccatatcgacgacgacgacaagcatatgctcgagaataagcaaagctccagaatatcaaatatagaaactgtgatagagttccaacaaaagaacaacagactactagagattaccagggaatttagtgttaatgcaggtgtaactacacctgtaagcacttacatgttaactaatagtgaattattgtcattaatcaatgatatgcctataacaaatgatcagaaaaagttaatgtccaacaatgttcaaatagttagacagcaaagttactctatcatgtccataataaaagaggaagtcttagcatatgtagtacaattaccactatatggtgtgatagatacaccttgttggaaattacacacatccccttcaattacaaccaacacaaaagaagggtcaaacatctgtttaacaagaactgacagaggatggtactgtgacaatgcaggatcagtatctttcttcccacaagctgaaacatgtaaagttcaatcgaatcgagtattttgtgacacaatgaacagtttaacattaccaagtgaagtaaatctctgcaatgttgacatattcaatcccaaatatgattgtaaaattatgacttcaaaaacagatgtaagcagctccgttatcacatctctaggagccattgtgtcatgctatggcaaaactaaatgtacagcatccaataaaaatcgtggaatcataaagacattttctaacgggtgtgattatgtatcaaataaaggggtggacactgtgtctgtaggtaacacattatattatgtaaataagcaagaaggcaaaagtctctatgtaaaaggtgaaccaataataaatttctatgacccattatag

SEQ ID NO: 41 - DNA, 903 bp, Artificial Sequence
  Recombinant polynucleotide encoding variant  F1 de15 C-V2′  RSV G protein
atgggccatcatcatcatcatcatcatcatcatcacagcagcggccatatcgacgacgacgacaagcatatgctcgagaataagcaaagctccagaatatcaaatatagaaactgtgatagagttccaacaaaagaacaacagactactagagattaccagggaatttagtgttaatgcaggtgtaactacacctgtaagcacttacatgttaactaatagtgaattattgtcattaatcaatgatatgcctataacaaatgatcagaaaaagttaatgtccaacaatgttcaaatagttagacagcaaagttactctatcatgtccataataaaagaggaagtcttagcatatgtagtacaattaccactatatggtgtgatagatacaccttcttggaaattacacacatccccttcaattacaaccaacacaaaagaagggtcaaacatctctttaacaagaactgacagaggatggtactctgacaatgcaggatcagtatctttcttcccacaagctgaaacatgtaaagttcaatcgaatcgagtattttgtgacacaatgaacagtttaacattaccaagtgaagtaaatctctgcaatgttgacatattcaatcccaaatatgattgtaaaattatgacttcaaaaacagatgtaagcagctccgttatcacatctctaggagccattgtgtcatgctatggcaaaactaaatgtacagcatccaataaaaatcgtggaatcataaagacattttctaacgggtctgattatgtatcaaataaaggggtggacactgtgtctgtaggtaacacattatattatgtaaataagcaagaaggcaaaagtctctatgtaaaaggtgaaccaataataaatttctatgacccattatag

SEQ ID NO: 42 - DNA, 396 bp, Artificial Sequence
  Recombinant polynucleotide encoding variant F2-pET19b RSV G protein
atgggccatcatcatcatcatcatcatcatcatcacagcagcggccatatcgacgacgacgacaagcatatgtctagtcaaaacatcactgaagaattttatcaatcaacatgcagtgcagttagcaaaggctatcttagtgctctaagaactggttggtatactagtgttataactatagaattaagtaatatcaaggaaaataagtgtaatggaacagatgctaaggtaaaattgatgaaacaagaattagataaatataaaaatgctgtaacagaattgcagttgctcatgcaaagcacaccagcagcaaacaatcgagccagaagagaactaccaaggtttatgaattatacactcaacaataccaaaaaaaccaatgtaacactaagctag

SEQ ID NO: 43 - DNA, 396 bp, Artificial Sequence
  Recombinant polynucleotide encoding variant  F2 C-V2′ RSV G protein
atgggccatcatcatcatcatcatcatcatcatcacagcagcggccatatcgacgacgacgacaagcatatgtctagtcaaaacatcactgaagaatttcttcaatcaataggcgttgcagttagcaaaggctatcttagtgctctaagaactggttggtatactagtgttataactatagaattaagtaatatcaaggaaaataagtctaatggaacagatgctaaggtaaaattgatgaaacaagaattagataaatataaaaatgctgtaacagaattgcagttgctcatgcaaagcacaccagcagcaaacaatcgagccagaagagaactaccaaggtttatgaattatacactcaacaataccaaaaaaaccaatgtaacactaagctag

SEQ ID NO: 44 - DNA, 1710 bp, Artificial Sequence
  Recombinant polynucleotide encoding Chimeric F2GF1 for CHO
aagcttgccaccatggagctgctgatcctgaaaaccaacgccatcaccgccatcctggccgccgtgaccctgtgcttcgcctcctcccagaacatcaccgaggagttctaccagtccacctgctccgccgtgtccaagggctacctgtccgccctgcggaccggctggtacacctccgtgatcaccatcgagctgtccaacatcaaggaaaacaagtgcaacggcaccgacgccaaggtgaagctgatcaagcaggagctggacaagtacaagagcgccgtgaccgaactccagctgctgatgcagtccacccctgccaccaacaaccgggccagacggggccagaacaagcctcctaacaagcccaacaacgacttccacttcgaggtgttcaacttcgtgccttgctccatctgctccaacaaccctacctgctgggccatctgcaagagaatccccaacaagaagcctggcaagaaaaccaccaccaagcctaccaagaagcctaccttcaagaccaccaagaaggaccacaagcctcagaccacaaagcctaaggaggtgccaaccaccaagggcatcgccgtgagcaaggtgctgcacctggagggcgaggtgaacaagatcaagagcgccctgctgtccaccaacaaggccgtggtgtccctgtccaacggcgtgtccgtgctgacctccaaggtgctggatctgaagaactacatcgacaagcagctgctgcctatcgtgaacaagcagtcctgctccatctccaacatcgagaccgtgatcgagttccagcagaagaacaaccggctgctggagatcacccgcgagttctccgtgaacgccggcgtgaccacccctgtgtccacctacatgctgaccaactccgagctgctgtccctgatcaacgacatgcctatcaccaacgaccagaaaaagctgatgtccaacaacgtgcagatcgtgcggcagcagtcctacagcatcatgagcatcatcaaggaagaggtgctggcctacgtggtgcagctgcctctgtacggcgtgatcgacaccccttgctggaagctgcacacctcccccctgtgcaccaccaacaccaaggagggctccaacatctgcctgacccggaccgaccggggctggtactgcgacaacgccggctccgtgtccttcttccctctggccgagacctgcaaggtgcagtccaaccgggtgttctgcgacaccatgaactccctgaccctgccttccgaggtgaacctgtgcaacatcgacatcttcaaccccaagtacgactgcaagatcatgaccagcaagaccgacgtgtcctccagcgtgatcacctccctgggcgccatcgtgtcctgctacggcaagaccaagtgcaccgcctccaacaagaaccggggaatcatcaagaccttctccaacggctgcgactacgtgtccaataagggcgtggacaccgtgtccgtgggcaacacactgtactacgtgaataagcaggagggcaagagcctgtacgtgaagggcgagcctatcatcaacttctacgaccctctggtgttcccttccgacgagttcgacgcctccatcagccaggtgaacgagaagatcaaccagtccctggccttcatccggaagtccgacgagctgctgcacaacgtgaacgctggcaagtccaccaccaactgataatctaga

SEQ ID NO: 45 - protein, 562 aa, Artificial Sequence
  Eukaryotic F2GF1 Chimeric Polypeptide
MELLILKTNAITAILAAVTLCFASSQNITEEFYQSTCSAVSKGYLSALRTGWYTSVITIELSNIKENKCNGTDAKVKLIKQELDKYKSAVTELQLLMQSTPATNNRARRGQNKPPNKPNNDFHFEVFNFVPCSICSNNPTCWAICKRIPNKKPGKKTTTKPTKKPTFKTTKKDHKPQTTKPKEVPTTKGIAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPLAETCKVQSNRVFCDTMNSLTLPSEVNLCNIDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTN

SEQ ID NO: 46 - DNA, 1704 bp, Artificial Sequence
  Recombinant polynucleotide encoding Eukaryotic F2GF1 delfur
aagcttgccaccatggagctgctgatcctgaaaaccaacgccatcaccgccatcctggccgccgtgaccctgtgcttcgcctcctcccagaacatcaccgaggagttctaccagtccacctgctccgccgtgtccaagggctacctgtccgccctgcggaccggctggtacacctccgtgatcaccatcgagctgtccaacatcaaggaaaacaagtgcaacggcaccgacgccaaggtgaagctgatcaagcaggagctggacaagtacaagagcgccgtgaccgaactccagctgctgatgcagtccacccctgccaccaacaacaaaaagggccagaacaagcctcctaacaagcccaacaacgacttccacttcgaggtgttcaacttcgtgccttgctccatctgctccaacaaccctacctgctgggccatctgcaagagaatccccaacaagaagcctggcaagaaaaccaccaccaagcctaccaagaagcctaccttcaagaccaccaagaaggaccacaagcctcagaccacaaagcctaaggaggtgccaaccaccaagggcatcgccgtgagcaaggtgctgcacctggagggcgaggtgaacaagatcaagagcgccctgctgtccaccaacaaggccgtggtgtccctgtccaacggcgtgtccgtgctgacctccaaggtgctggatctgaagaactacatcgacaagcagctgctgcctatcgtgaacaagcagtcctgctccatctccaacatcgagaccgtgatcgagttccagcagaagaacaaccggctgctggagatcacccgcgagttctccgtgaacgccggcgtgaccacccctgtgtccacctacatgctgaccaactccgagctgctgtccctgatcaacgacatgcctatcaccaacgaccagaaaaagctgatgtccaacaacgtgcagatcgtgcggcagcagtcctacagcatcatgagcatcatcaaggaagaggtgctggcctacgtggtgcagctgcctctgtacggcgtgatcgacaccccttgctggaagctgcacacctcccccctgtgcaccaccaacaccaaggagggctccaacatctgcctgacccggaccgaccggggctggtactgcgacaacgccggctccgtgtccttcttccctctggccgagacctgcaaggtgcagtccaaccgggtgttctgcgacaccatgaactccctgaccctgccttccgaggtgaacctgtgcaacatcgacatcttcaaccccaagtacgactgcaagatcatgaccagcaagaccgacgtgtcctccagcgtgatcacctccctgggcgccatcgtgtcctgctacggcaagaccaagtgcaccgcctccaacaagaaccggggaatcatcaagaccttctccaacggctgcgactacgtgtccaataagggcgtggacaccgtgtccgtgggcaacacactgtactacgtgaataagcaggagggcaagagcctgtacgtgaagggcgagcctatcatcaacttctacgaccctctggtgttcccttccgacgagttcgacgcctccatcagccaggtgaacgagaagatcaaccagtccctggccttcatccggaagtccgacgagctgctgcacaacgtgaacgctggcaagtccaccaccaactgataatctaga

SEQ ID NO: 47 - protein, 560 aa, Artificial Sequence
  Eukaryotic F2GF1 delfur
MELLILKTNAITAILAAVTLCFASSQNITEEFYQSTCSAVSKGYLSALRTGWYTSVITIELSNIKENKCNGTDAKVKLIKQELDKYKSAVTELQLLMQSTPATNNKKGQNKPPNKPNNDFHFEVFNFVPCSICSNNPTCWAICKRIPNKKPGKKTTTKPTKKPTFKTTKKDHKPQTTKPKEVPTTKGIAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPLAETCKVQSNRVFCDTMNSLTLPSEVNLCNIDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTN

(19) United States
(12) Patent Application Publication
(10) Pub. No.: US 2010/0203071 A1
(43) Pub. Date: Aug. 12, 2010

(54) CHIMERIC ANTIGENS

(76) Inventors: Norman Blais, Laval (CA); David S. Burt, Laval (CA); Sonya L. Cyr, Laval (CA); Denis L. Martin, Laval (CA); Patrick Rheault, Laval (CA)

Correspondence Address: GlaxoSmithKline;GLOBAL PATENTS -US, UW2220, P. O. BOX 1539, KING OF PRUSSIA, PA, 19406-0939, US

(21) Appl. No.: 12/531,758

(22) PCT Filed: Mar. 20, 2008
(86) PCT No.: PCT/IB08/01286

§ 371 Date: Sep. 17, 2009

Related U.S. Application Data
(60) Provisional application No. 60/896,201, filed on Mar. 21, 2007.

(51) Int. Cl. A61K 39/12; C07K 14/005; C07H 21/04; C12N 5/10

(52) U.S. Cl. 424/186.1; 530/350; 536/23.72; 435/325

(57) ABSTRACT

Chimeric respiratory syncytial virus (RSV) polypeptide antigens are provided. The disclosed polypeptides include in an N-terminal to C-terminal direction: a first F protein polypeptide domain; a G protein polypeptide domain; and a second F protein polypeptide domain. The disclosure also provides nucleic acids that encode, and pharmaceutical compositions that contain, the chimeric RSV polypeptides, as well as methods for their production and use.

### CROSS-REFERENCE TO RELATED APPLICATIONS

This application claims benefit of the filing date of U.S. Provisional Application No. 60/896,201, filed 21 Mar. 2007, the disclosure of which is incorporated herein by reference in its entirety.

### COPYRIGHT NOTIFICATION PURSUANT TO 37 C.F.R. §1.71(E)

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

### FIELD

This disclosure concerns the field of immunology. More particularly, this disclosure relates to compositions and methods for eliciting an immune response specific for Respiratory Syncytial Virus (RSV).

### BACKGROUND

Human Respiratory Syncytial Virus (RSV) is the most common worldwide cause of lower respiratory tract infections (LRI) in infants less than 6 months of age and premature babies less than or equal to 35 weeks of gestation. The RSV disease spectrum includes a wide array of respiratory symptoms from rhinitis and otitis to pneumonia and bronchiolitis, the latter two diseases being associated with considerable morbidity and mortality. Humans are the only known reservoir for RSV. Spread of the virus from contaminated nasal secretions occurs via large respiratory droplets, so close contact with an infected individual or contaminated surface is required for transmission. RSV can persist for several hours on toys or other objects, which explains the high rate of nosocomial RSV infections, particularly in paediatric wards.

The global annual infection and mortality figures for RSV are estimated to be 64 million and 160,000 respectively. In the U.S. alone RSV is estimated to be responsible for 18,000 to 75,000 hospitalizations and 90 to 1900 deaths annually. In temperate climates, RSV is well documented as a cause of yearly winter epidemics of acute LRI, including bronchiolitis and pneumonia. In the USA, nearly all children have been infected with RSV by two years of age. The incidence rate of RSV-associated LRI in otherwise healthy children was calculated as 37 per 1000 child-year in the first two years of life (45 per 1000 child-year in infants less than 6 months old) and the risk of hospitalization as 6 per 1000 child-years (11 per 1000 child-years in the first six months of life). Incidence is higher in children with cardio-pulmonary disease and in those born prematurely, who constitute almost half of RSV-related hospital admissions in the USA. Children who experience a more severe LRI caused by RSV later have an increased incidence of childhood asthma. The costs of caring for children with severe LRI and their sequelae are substantial, and RSV is also increasingly recognized as a important cause of morbidity from influenza-like illness in the elderly, highlighting the need for a safe and effective vaccine capable of protecting against RSV-induced disease.

### SUMMARY

This disclosure concerns chimeric respiratory syncytial virus (RSV) antigens. The chimeric RSV antigens include, in an N-terminal to C-terminal direction: a first F protein polypeptide domain; a G protein polypeptide domain; and a second F protein polypeptide domain. The disclosed antigens elicit an immune response when administered to a subject, and can be used to treat and/or prevent the symptoms of RSV infection. Also disclosed are nucleic acids that encode the chimeric antigens, immunogenic compositions that contain the chimeric antigens, and methods for producing and using the chimeric antigens.

### BRIEF DESCRIPTION OF THE DRAWINGS

FIG. 1A is a schematic illustration highlighting structural features of the RSV F protein (574 amino acids). FIG. 1B is a schematic illustration highlighting structural features of the RSV G protein (298 amino acids). FIG. 1C is a schematic illustration highlighting structural features of an exemplary eukaryotic F2GF1 chimeric RSV antigen (562 amino acids).

FIG. 2 is a schematic illustration of exemplary F2GF1 chimeric RSV antigens.

FIG. 3 schematically illustrates an exemplary expression construct including a polynucleotide sequence that encodes a F2GF1 chimeric RSV antigen.

FIGS. 4A-L are a sequence alignment illustrating similarity and variation between F proteins of different strains (or isolates) of RSV.

FIGS. 5A-QQ are a sequence alignment illustrating similarity and variation between G proteins of different strains (or isolates) of RSV.

FIG. 6 is a bar graph illustrating human sera neutralization by F2GF1 chimeric RSV antigen.

FIG. 7 is a graph showing protection against RSV following administration of F2GF1 chimeric antigen.

FIG. 8 is a bar graph showing serum neutralization by antibodies elicited by immunization with F2GF1 chimeric antigen.

### DESCRIPTION OF THE SEQUENCE LISTING

SEQ ID NO:1: Nucleotide sequence of RSV Long strain Fusion (F) protein.

SEQ ID NO:2: Amino acid sequence of RSV Long strain Fusion (F) protein.

SEQ ID NO:3: Nucleotide sequence of RSV Long strain G protein.

SEQ ID NO:4: Amino acid sequence of RSV Long strain G protein.

SEQ ID NO:5: Nucleotide sequence encoding P3-1 chimeric F2GF1 polypeptide. Nucleotides 1 to 78 are from the vector and include a 10 histidines N-terminal tag. Nucleotides 79 to 399 encode amino acids 24 to 130 of the F0 protein (F2). Nucleotides 406 to 711 encode amino acids 128 to 229 of the G protein. Nucleotides 718 to 1809 encode amino acids 161 to 524 of the F0 protein. Two 6 nucleotides bridges between the F and the G regions at position 400 to 405 and 712 to 717 were generated to link each fragment together. Both bridges code for 2 glycine amino acid residues.

SEQ ID NO:6: Amino acid sequence of P3-1 F2GF1 polypeptide Amino acids 1 to 26 are from the vector and include a 10 histidines N-terminal tag. Amino acids 27 to 133 correspond to the amino acids 24 to 130 of the F0 protein (F2). Amino acids 136 to 237 correspond to the amino acids 128 to 229 of the G protein. Amino acids 240 to 603 correspond to the amino acids 161 to 524 of the F0 protein. Linkers between the F and the G regions are located at position 134 to 135 and 238 to 239.

SEQ ID NO:7: Nucleotide sequence encoding P3-2 chimeric F2GF1 polypeptide. Nucleotides 1 to 78 are from the vector and include a 10 histidines N-terminal tag. Nucleotides 79 to 330 encode amino acids 24 to 107 of the F0 protein (F2). Nucleotides 337 to 579 encode amino acids 149 to 229 of the G protein. Nucleotides 586 to 1677 encode amino acids 161 to 524 of the F0 protein. Two 6 nucleotides bridges between the F and the G regions at position 331 to 336 and 580 to 585 were generated to link each fragment together. Both bridges code for 2 glycine amino acid residues.

SEQ ID NO:8: Amino acid sequence of P3-2 F2GF1 polypeptide Amino acids 1 to 26 are from the vector and include a 10 histidines N-terminal tag. Amino acids 27 to 110 correspond to the amino acids 24 to 107 of the F0 protein (F2). Amino acids 113 to 193 correspond to the amino acids 149 to 229 of the G protein. Amino acids 196 to 559 correspond to the amino acids 161 to 524 of the F0 protein (F1). Linkers between the F and the G regions are located at position 111 to 112 and 194 to 195.

SEQ ID NO:9: Nucleotide sequence encoding P3-3 chimeric F2GF1 polypeptide. Amino acids 1 to 26 are from the vector and include a 10 histidines N-terminal tag Amino acids 27 to 110 correspond to the amino acids 24 to 107 of the F0 protein (F2) Amino acids 113 to 193 correspond to the amino acids 149 to 229 of the G protein Amino acids 196 to 559 correspond to the amino acids 161 to 524 of the F0 protein (F1). Linkers between the F and the G regions are located at position 111 to 112 and 194 to 195.

SEQ ID NO:10: Amino acid sequence of P3-3 F2GF1 polypeptide. Amino acids 1 to 26 are from the vector and include a 10 histidines N-terminal tag. Amino acids 27 to 110 correspond to the amino acids 24 to 107 of the F0 protein (F2). Amino acids 113 to 214 correspond to the amino acids 128 to 229 of the G protein. Amino acids 217 to 580 correspond to the amino acids 161 to 524 of the F0 protein (F1). Linkers between the F and the G regions are located at position 111 to 112 and 215 to 216.

SEQ ID NO:11: Nucleotide sequence encoding P3-4 chimeric F2GF1 polypeptide. Nucleotides 1 to 78 are from the vector and include a 10 histidines N-terminal tag. Nucleotides 79 to 399 encode amino acids 24 to 130 of the F0 protein (F2). Nucleotides 406 to 648 encode amino acids 149 to 229 of the G protein. Nucleotides 655 to 1746 encode amino acids 161 to 524 of the F0 protein. Two 6 nucleotides bridges between the F and the G regions at position 400 to 405 and 649 to 654 were generated to link each fragment together. Both bridges code for 2 glycine amino acid residues.

SEQ ID NO:12: Amino acid sequence of P3-4 F2GF1 polypeptide Amino acids 1 to 26 are from the vector and include a 10 histidines N-terminal tag. Amino acids 27 to 133 correspond to the amino acids 24 to 130 of the F0 protein (F2). Amino acids 136 to 216 correspond to the amino acids 149 to 229 of the G protein. Amino acids 219 to 582 correspond to the amino acids 161 to 524 of the F0 protein. Linkers between the F and the G regions are located at position 134 to 135 and 217 to 218.

SEQ ID NO: 13: Nucleotide sequence encoding P3-5 chimeric F2GF1 polypeptide. Nucleotides 1 to 78 are from the vector and include a 10 histidines N-terminal tag. Nucleotides 79 to 399 encode amino acids 24 to 130 of the F0 protein (F2). Nucleotides 406 to 711 encode amino acids 128 to 229 of the G protein. Nucleotides 718 to 1809 encode amino acids 161 to 524 of the F0 protein. Two 6 nucleotides bridges between the F and the G regions at position 400 to 405 and 712 to 717 were generated to link each fragment together. Both bridges code for 2 glycine amino acid residues.

SEQ ID NO:14: Amino acid sequence of P3-5 F2GF1 polypeptide. Amino acids 1 to 26 are from the vector and include a 10 histidines N-terminal tag. Amino acids 27 to 133 correspond to the amino acids 24 to 130 of the F0 protein (F2). Amino acids 136 to 237 correspond to the amino acids 128 to 229 of the G protein. Amino acids 240 to 603 correspond to the amino acids 161 to 524 of the F0 protein. Linkers between the F and the G regions are located at position 134 to 135 and 238 to 239.

SEQ ID NO:15: Nucleotide sequence encoding P3-6 chimeric F2GF1 polypeptide. Nucleotides 1 to 78 are from the vector and include a 10 histidines N-terminal tag. Nucleotides 79 to 330 encode amino acids 24 to 107 of the F0 protein (F2). Nucleotides 337 to 579 encode amino acids 149 to 229 of the G protein. Nucleotides 586 to 1677 encode amino acids 161 to 524 of the F0 protein. Two 6 nucleotides bridges between the F and the G regions at position 331 to 336 and 580 to 585 were generated to link each fragment together. Both bridges code for 2 glycine amino acid residues.

SEQ ID NO:16: Amino acid sequence of P3-6 F2GF1 polypeptide. Amino acids 1 to 26 are from the vector and include a 10 histidines N-terminal tag. Amino acids 27 to 110 correspond to the amino acids 24 to 107 of the F0 protein (F2). Amino acids 113 to 193 correspond to the amino acids 149 to 229 of the G protein. Amino acids 196 to 559 correspond to the amino acids 161 to 524 of the F0 protein (F1). Linkers between the F and the G regions are located at position 111 to 112 and 194 to 195.

SEQ ID NO:17: Nucleotide sequence encoding P3-7 F2GF1 polypeptide. Nucleotides 1 to 78 are from the vector and include a 10 histidines N-terminal tag. Nucleotides 79 to 330 encode amino acids 24 to 107 of the F0 protein (F2). Nucleotides 337 to 642 encode amino acids 128 to 229 of the G protein. Nucleotides 649 to 1740 encode amino acids 161 to 524 of the F0 protein. Two 6 nucleotides bridges between the F and the G regions at position 331 to 336 and 643 to 648 were generated to link each fragment together. Both bridges code for 2 glycine amino acid residues.

SEQ ID NO:18: Amino acid sequence of P3-7 F2GF1 polypeptide Amino acids 1 to 26 are from the vector and include a 10 histidines N-terminal tag Amino acids 27 to 110 correspond to the amino acids 24 to 107 of the F0 protein (F2). Amino acids 113 to 214 correspond to the amino acids 128 to 229 of the G protein. Amino acids 217 to 580 correspond to the amino acids 161 to 524 of the F0 protein (F1). Linkers between the F and the G regions are located at position 111 to 112 and 215 to 216.

SEQ ID NO:19: Nucleotide sequence encoding P3-8 F2GF1 polypeptide. Nucleotides 1 to 78 are from the vector and include a 10 histidines N-terminal tag. Nucleotides 79 to 399 encode amino acids 24 to 130 of the F0 protein (F2). Nucleotides 406 to 648 encode amino acids 149 to 229 of the G protein. Nucleotides 655 to 1746 encode amino acids 161 to 524 of the F0 protein. Two 6 nucleotides bridges between the F and the G regions at position 400 to 405 and 649 to 654 were generated to link each fragment together. Both bridges code for 2 glycine amino acid residues.

SEQ ID NO:20: Amino acid sequence of P3-8 F2GF1 polypeptide. Amino acids 1 to 26 are from the vector and include a 10 histidines N-terminal tag. Amino acids 27 to 133 correspond to the amino acids 24 to 130 of the F0 protein (F2). Amino acids 136 to 216 correspond to the amino acids 149 to 229 of the G protein. Amino acids 219 to 582 correspond to the amino acids 161 to 524 of the F0 protein. Linkers between the F and the G regions are located at position 134 to 135 and 217 to 218.

SEQ ID NO:21: Nucleotide sequence encoding F2GF1-1 C-V1 (SEQ ID NO:22 is the encoded amino acid sequence). Nucleotides 1 to 78 are from the vector and include a 10 histidines N-terminal tag. Nucleotides 79 to 399 encode amino acids 24 to 130 of the F0 protein (F2). Nucleotides 406 to 711 encode amino acids 128 to 229 of the G protein. Nucleotides 718 to 1809 encode amino acids 161 to 524 of the F0 protein. Two 6 nucleotides bridges between the F and the G regions at position 400 to 405 and 712 to 717 were generated to link each fragment together. Both bridges code for 2 glycine amino acid residues. Four altered codons encode cysteine to serine substitutions at nucleotide positions: 1175, 1235, 1265 and 1553 (amino acid residues 392, 412, 422 and 518).

SEQ ID NO:23: Nucleotide sequence encoding F2GF1-1 C-V2 (SEQ ID NO:24 is the encoded amino acid sequence). Nucleotides 1 to 78 are from the vector and include a 10 histidines N-terminal tag. Nucleotides 79 to 399 encode amino acids 24 to 130 of the F0 protein (F2). Nucleotides 406 to 711 encode amino acids 128 to 229 of the G protein. Nucleotides 718 to 1809 encode amino acids 161 to 524 of the F0 protein. Two 6 nucleotides bridges between the F and the G regions at position 400 to 405 and 712 to 717 were generated to link each fragment together. Both bridges code for 2 glycine amino acid residues. Four altered condons encode cysteine to serine substitutions at nucleotide positions: 119, 215, 872 and 1202 (amino acid residues 40, 72, 291 and 401).

SEQ ID NO:25: Nucleotide sequences encoding F2GF1-1 C-V12 (SEQ ID NO:26 is the encoded amino acid sequence). Nucleotides 1 to 78 are from the vector and include a 10 histidines N-terminal tag. Nucleotides 79 to 399 encode amino acids 24 to 130 of the F0 protein (F2). Nucleotides 406 to 711 encode amino acids 128 to 229 of the G protein. Nucleotides 718 to 1809 encode amino acids 161 to 524 of the F0 protein. Two 6 nucleotides bridges between the F and the G regions at position 400 to 405 and 712 to 717 were generated to link each fragment together. Both bridges code for 2 glycine amino acid residues. Eight altered codons encode cysteine to serine substitutions at positions nucleotide positions 119, 215, 872, 1175, 1202, 1235, 1265 and 1553 (amino acid residues 40, 72, 291, 392, 401, 412, 422 and 518).

SEQ ID NO:27: Nucleotide sequences encoding F2GF1-1 C-V12′ (SEQ ID NO:28 is the encoded amino acid sequence). Nucleotides 1 to 78 are from the vector and include a 10 histidines N-terminal tag. Nucleotides 79 to 399 encode amino acids 24 to 130 of the F0 protein (F2). Nucleotides 406 to 711 encode amino acids 128 to 229 of the G protein. Nucleotides 718 to 1809 encode amino acids 161 to 524 of the F0 protein. Two 6 nucleotides bridges between the F and the G regions at position 400 to 405 and 712 to 717 were generated to link each fragment together. Both bridges code for 2 glycine amino acid residues. Twelve altered codons encode cysteine to serine substitutions at nucleotide positions 106, 107, 116, 118, 121, 122, 215, 872, 1175, 1198, 1199, 1201, 1202, 1235, 1265 and 1553.

SEQ ID NO:29: Nucleotide sequence encoding F2GF1-1 del1 (SEQ ID NO:30 is the encoded amino acid sequence). This is a version of F2GF1-1 in which a F1 portion was truncated to delete the first 47 amino acids of F1. Nucleotides 1 to 78 are from the vector and include a 10 histidines N-terminal tag. Nucleotides 79 to 399 encode amino acids 24 to 130 of the F0 protein (F2). Nucleotides 406 to 711 encode amino acids 128 to 229 of the G protein. Nucleotides 718 to 1668 encode amino acids 208 to 524 of the F0 protein.

SEQ ID NO:31: Nucleotide sequence encoding F2GF1-1 del2 (SEQ ID NO:32 is the encoded amino acid sequence). This is a version of F2GF1-1 in which a F1 portion was truncated to delete the first 42 amino acids of the F1. Nucleotides 1 to 78 are from the vector and include a 10 histidines N-terminal tag. Nucleotides 79 to 399 encode amino acids 24 to 130 of the F0 protein (F2). Nucleotides 406 to 711 encode amino acids 128 to 229 of the G protein. Nucleotides 718 to 1683 encode amino acids 203 to 524 of the F0 protein.

SEQ ID NO:33: Nucleotide sequence encoding F2GF1-1 del3 (SEQ ID NO:34 is the encoded amino acid sequence). This is a version of F2GF1-1 in which a F1 portion was truncated to delete the 24 first amino acids of the F1 are deleted. Nucleotides 1 to 78 are from the vector and include a 10 histidines N-terminal tag. Nucleotides 79 to 399 encode amino acids 24 to 130 of the F0 protein (F2). Nucleotides 406 to 711 encode amino acids 128 to 229 of the G protein. Nucleotides 718 to 1737 encode amino acids 185 to 524 of the F0 protein.

SEQ ID NO:35: Nucleotide sequence encoding F2GF1-1 del4 (SEQ ID NO:36 is the encoded amino acid sequence). This is a version of F2GF1-1 in which a F1 portion was truncated. Nucleotides 1 to 78 are from the vector and include a 10 histidines N-terminal tag. Nucleotides 79 to 399 encode amino acids 24 to 130 of the F0 protein (F2). Nucleotides 406 to 711 encode amino acids 128 to 229 of the G protein. Nucleotides 718 to 1677 encode amino acids 205 to 524 of the F0 protein.

SEQ ID NO:37: Nucleotide sequence encoding F2GF1-1 del5 (SEQ ID NO:38 is the encoded amino acid sequence). This is a version of F2GF1-1 in which both extremities of the F1 portion were truncated. Nucleotides 1 to 78 are from the vector and include a 10 histidines N-terminal tag. Nucleotides 79 to 399 encode amino acids 24 to 130 of the F0 protein (F2). Nucleotides 406 to 711 encode amino acids 128 to 229 of the G protein. Nucleotides 718 to 1545 encode amino acids 206 to 481 of the F0 protein.

SEQ ID NO:39: Nucleotide sequence encoding F2GF1-1 del6 (SEQ ID NO:40 is the encoded amino acid sequence). This is a version of F2GF1-1 in which both extremities of the F1 portion were truncated. Nucleotides 1 to 78 are from the vector and include a 10 histidines N-terminal tag. Nucleotides 79 to 399 encode amino acids 24 to 130 of the F0 protein (F2). Nucleotides 406 to 711 encode amino acids 128 to 229 of the G protein. Nucleotides 718 to 1569 encode amino acids 206 to 481 of the F0 protein.

SEQ ID NO:41: Nucleotide sequence encoding F2GF1-1 del5 C-V12 (SEQ ID NO:42 is the encoded amino acid sequence). This is a version of F2GF1-1 in which both extremities of the F1 portion were truncated. 8 codons were also modified at nucleotide positions 119, 215, 737, 1040, 1067, 1100, 1130 and 1418. It is a combination of the del5 and C-V12 modifications. Nucleotides 1 to 78 are from the vector and include a 10 histidines N-terminal tag. Nucleotides 79 to 399 encode amino acids 24 to 130 of the F0 protein (F2). Nucleotides 406 to 711 encode amino acids 128 to 229 of the G protein. Nucleotides 718 to 1545 encode amino acids 206 to 481 of the F0 protein. The modified codons are highlighted.

SEQ ID NO:43: Nucleotide sequence encoding F2GF1-1 del6 C-V12 (SEQ ID NO:44 is the encoded amino acid sequence). This is a version of F2GF1-1 in which both extremities of the F1 portion were truncated. 8 codons were also modified at the nucleotide positions 119, 215, 755, 1058, 1085, 1118, 1148 and 1436. It is a combinaison of the del6 and C-V12 modifications. Nucleotides 1 to 78 are from the vector and include a 10 histidines N-terminal tag. Nucleotides 79 to 399 encode amino acids 24 to 130 of the F0 protein (F2). Nucleotides 406 to 711 encode amino acids 128 to 229 of the G protein. Nucleotides 718 to 1569 encode amino acids 206 to 481 of the F0 protein.

SEQ ID NO:45: Nucleotide sequence encoding An-G polypeptide (SEQ ID NO:46 is the encoded amino acid sequence). Nucleotides 1 to 72 encode N-terminal histidine tag. Nucleotides 73 to 378 encode amino acids 128 to 229 of the G protein.

SEQ ID NO:47: Nucleotide sequence encoding An-G-0 polypeptide (SEQ ID NO:48 is the encoded amino acid sequence). Codon optimized G protein polypeptide. Nucleotides 1 to 72 encode N-terminal histidine tag. Nucleotides 73 to 378 encode amino acids 128 to 229 of the G protein.

SEQ ID NO:49: Nucleotide sequence encoding An-GT polypeptide (SEQ ID NO:50 is the encoded amino acid sequence). Nucleotides 1 to 72 encode N-terminale histidine tag. Nucleotides 73 to 312 encode amino acids 149 to 229 of the G protein.

SEQ ID NO:51: Nucleotide sequence encoding An-GT-O polypeptide (SEQ ID NO:52 is the encoded amino acid sequence). Nucleotides 1 to 72 encode N-terminale histidine tag. Nucleotides 73 to 312 encode amino acids 149 to 229 of the G protein.

SEQ ID NO:53: Nucleotide sequence encoding F1 polypeptide (SEQ ID NO:54 is the encoded amino acid sequence). Nucleotides 1 to 78 are part the vector (pET19b) and include a 10 histidines N-terminal tag. Nucleotides 79 to 1158 encode amino acids 162 to 524 of the F0 protein.

SEQ ID NO:55: Nucleotide sequence encoding F1 del5 (SEQ ID NO:56 is the encoded amino acid sequence). Version of the F1 polypeptide truncated at both extremities of the F1 coding sequence. Nucleotides 1 to 78 are parts the vector (pET19b) and includes a 10 histidines N-terminal tag. Nucleotides 79 to 900 encode amino acids 208 to 481 of the F0 protein.

SEQ ID NO:57: Nucleotide sequence encoding F1 del5 C-V1 (SEQ ID NO:58 is the encoded amino acid sequence). This version of the F polypeptide is similar to F1 del5. Four codons were altered to generate 4 cysteine to serine point mutations.

SEQ ID NO:59: Nucleotide sequence encoding F1 del5 C-V2′ (SEQ ID NO:60 is the encoded amino acid sequence). This version of the F polypeptide is similar to F1 del5. Three codons were altered to generate 3 point mutations.

SEQ ID NO:61: Nucleotide sequence encoding F1 del5 C-V12′ (SEQ ID NO:62 is the encoded amino acid sequence). This version of the F polypeptide is similar to F1 del5. Seven codons were changed to generate point mutations, combining the substitutions of F1 del5 C-V1 and F1 del5 C-V2′ together.

SEQ ID NO:63: Nucleotide sequence encoding F2 polypeptide (SEQ ID NO:64 is the encoded amino acid sequence). Nucleotides 1 to 72 are from the vector (pET19b) and includes a 10 histidines N-terminal tag. Nucleotides 73 to 393 encode amino acids 24 to 130 of the F0 protein.

SEQ ID NO:65: Nucleotide sequence encoding F2 C-V2′ (SEQ ID NO:66 is the encoded amino acid sequence). This version is similar to F2 (SEQ ID NO:41). Five codons were changed to generate point mutations.

SEQ ID NO:67: Nucleotide sequence encoding an exemplary eukaryotic chimeric F2GF1 polypeptide.

SEQ ID NO:68: Amino acid sequence of eukaryotic chimeric F2GF1 polypeptide.

SEQ ID NO:69: Nucleotide sequence encoding an exemplary eukaryotic chimeric F2GF1 polypeptide with a deletion of the furin cleavage sites (eukaryotic F2GF1 delfur).

SEQ ID NO:70: Amino acid sequence of eukaryotic F2GF1 delfur.

### DETAILED DESCRIPTION

### Introduction

Development of vaccines that protect against the symptoms and sequelae caused by RSV infection has been complicated by the fact that host immune responses appear to play a role in the pathogenesis of the disease. Early studies in the 1960s showed that children vaccinated with a formalin-inactivated RSV vaccine suffered from more severe disease on subsequent exposure to the virus as compared to unvaccinated control subjects. These early trials resulted in the hospitalization of 80% of vaccinees and two deaths. The enhanced severity of disease has been reproduced in animal models and is thought to result from inadequate levels of serum-neutralizing antibodies, lack of local immunity, and excessive induction of a type 2 helper T-cell-like (Th2) immune response with pulmonary eosinophilia and increased production of IL-4 and IL-5 cytokines. In contrast, a successful vaccine that protects against RSV infection induces a Th1-type immune response, characterized by production of IL-2 and γ-interferon (IFN-γ).

Various approaches have been attempted in efforts to produce a safe and effective RSV vaccine that produces durable and protective immune responses in healthy and at risk populations. However, none of the candidate evaluated to date have proven safe and effective as vaccines for the purpose of preventing RSV infection and/or reducing or preventing RSV disease, including lower respiratory infections (LRIs).

The present disclosure concerns chimeric RSV antigens that include the predominant immunoprotective epitope of the G protein internally positioned within the RSV F protein polypeptide, such that a readily soluble chimeric RSV antigen can be produced in a recombinant expression system. These novel chimeric RSV antigens overcome several significant drawbacks encountered in previous attempts to produce safe and effective chimeric RSV antigens that are suitable for administration as prophylactic and therapeutic vaccines.

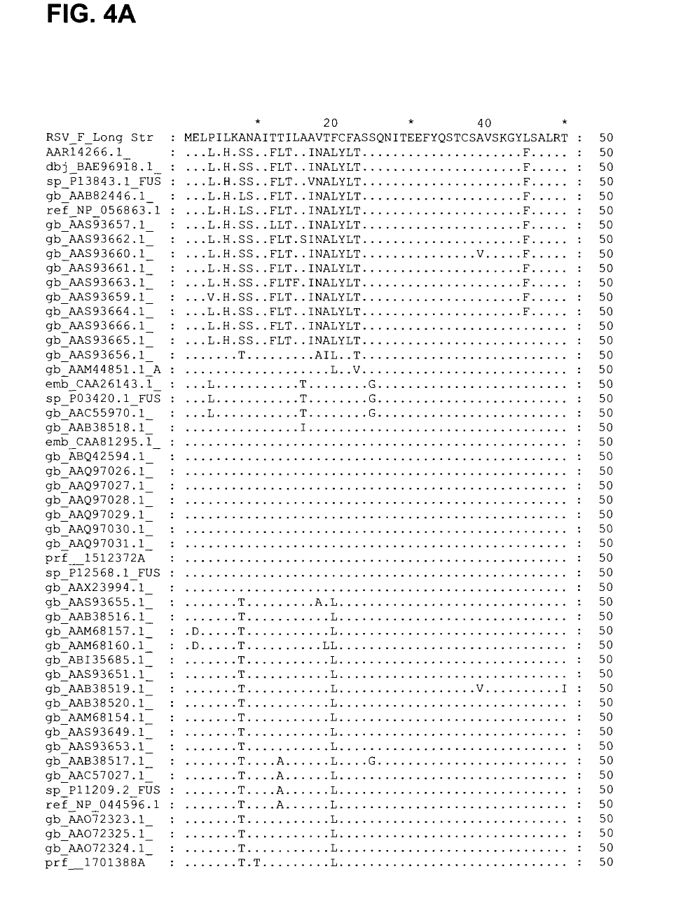

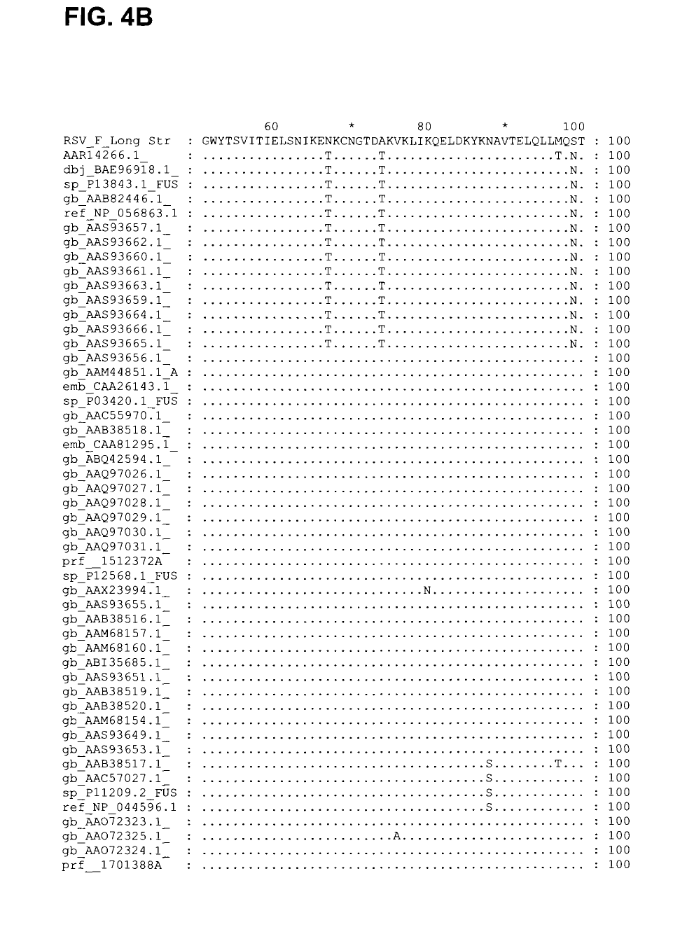

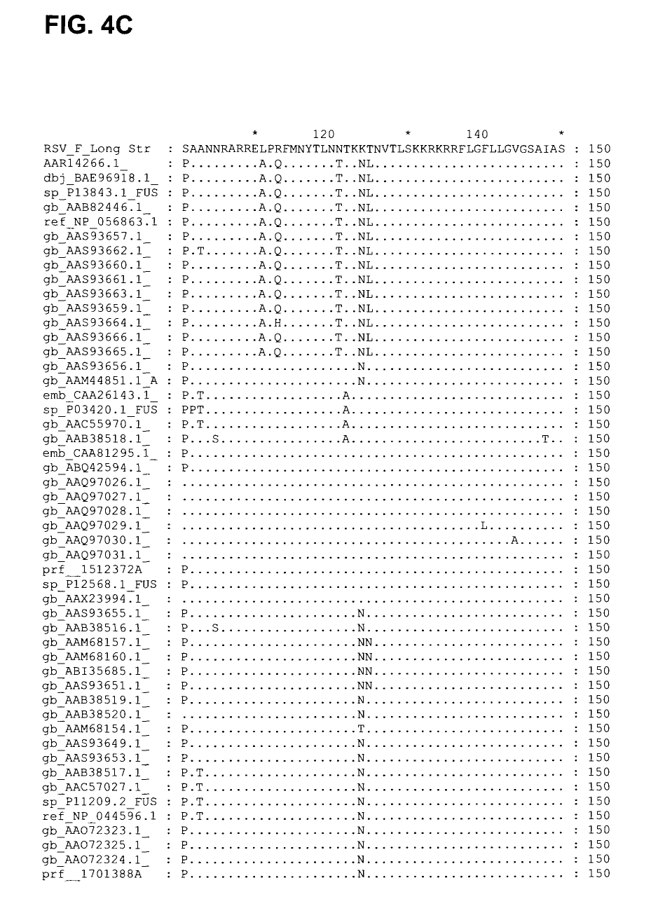

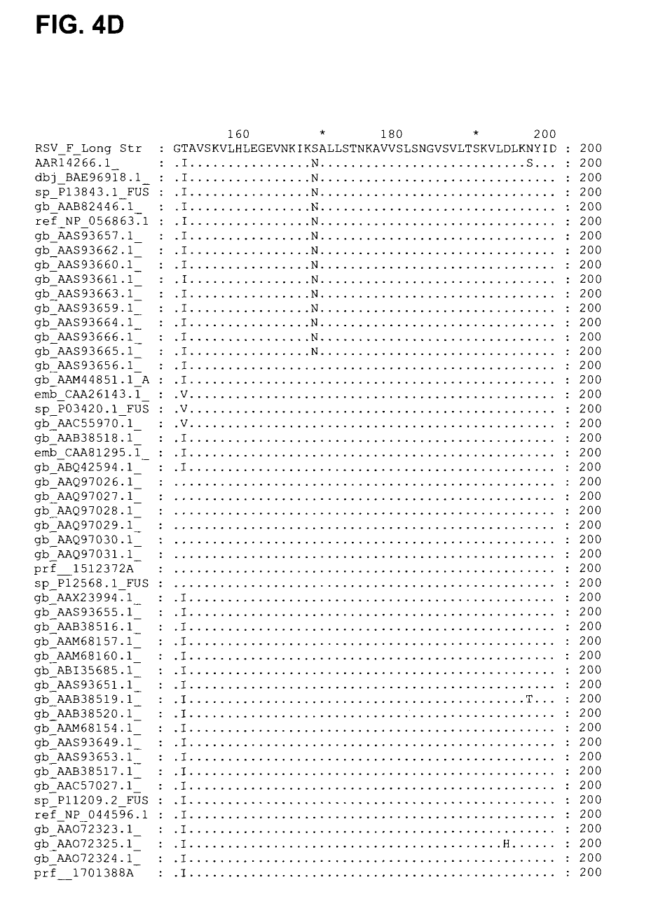

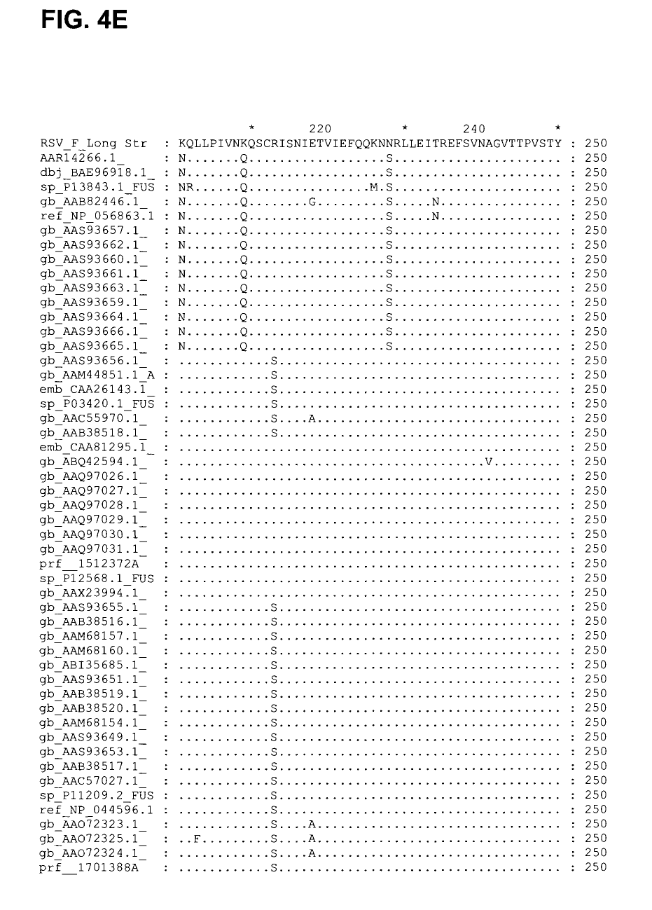

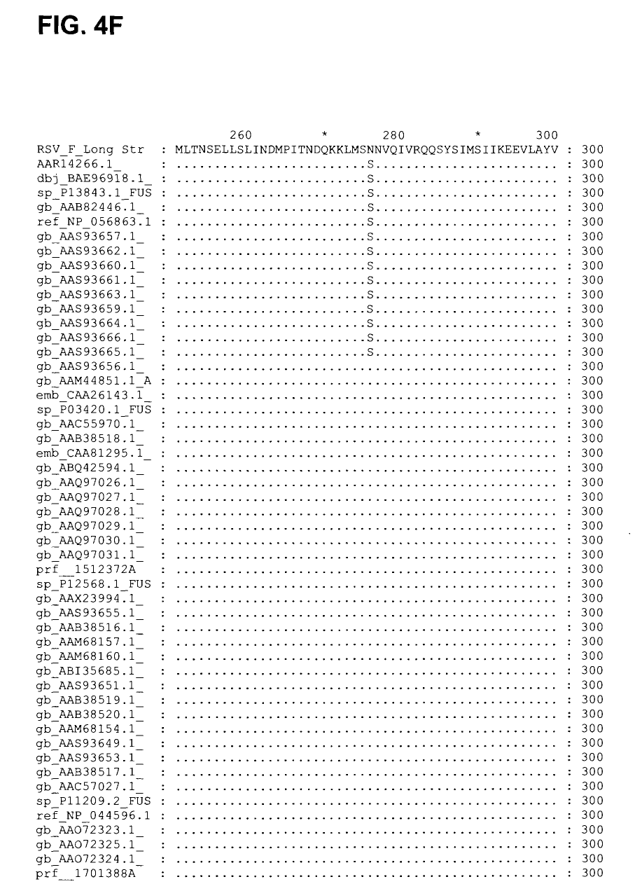

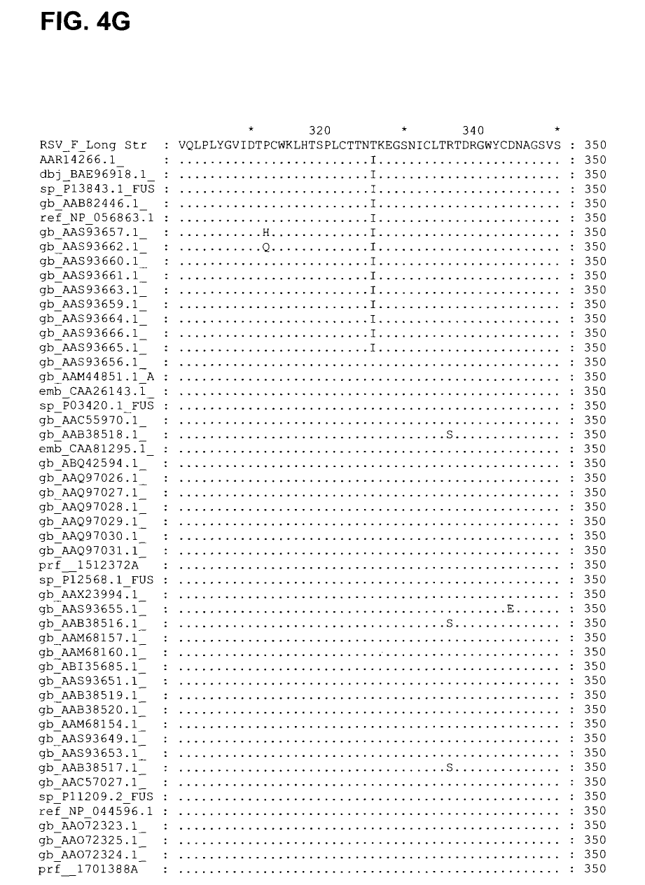

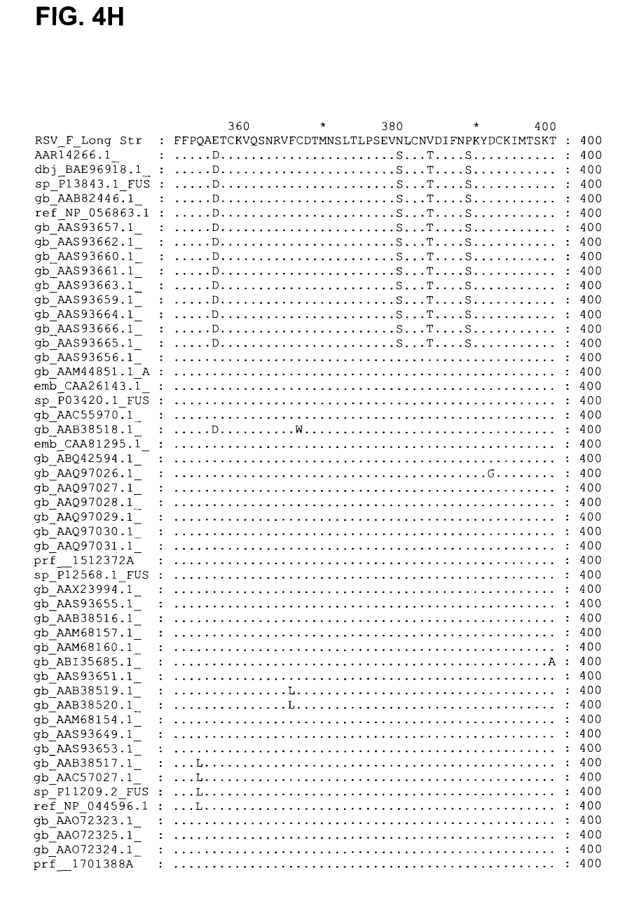

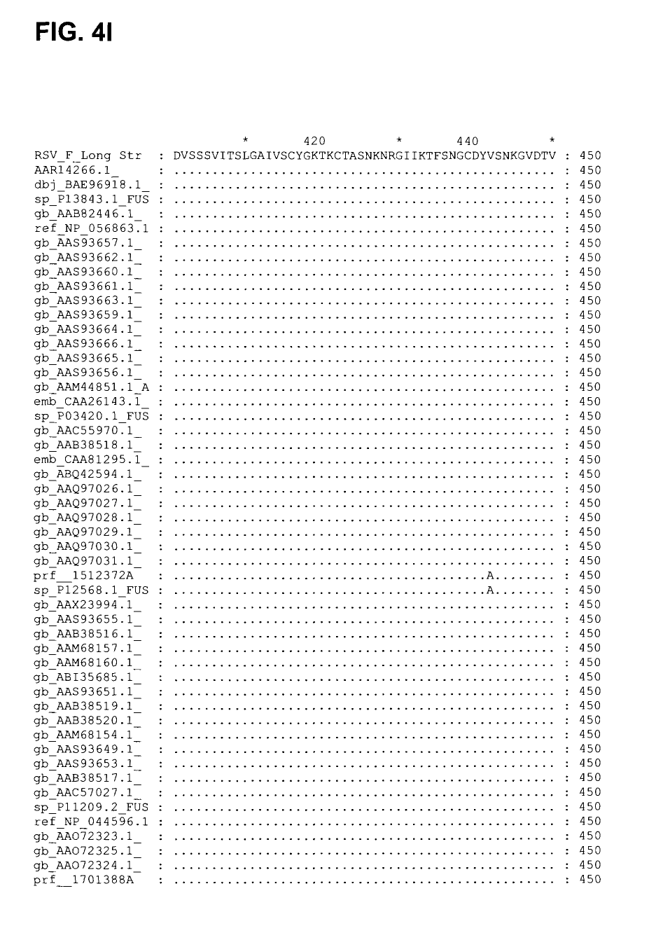

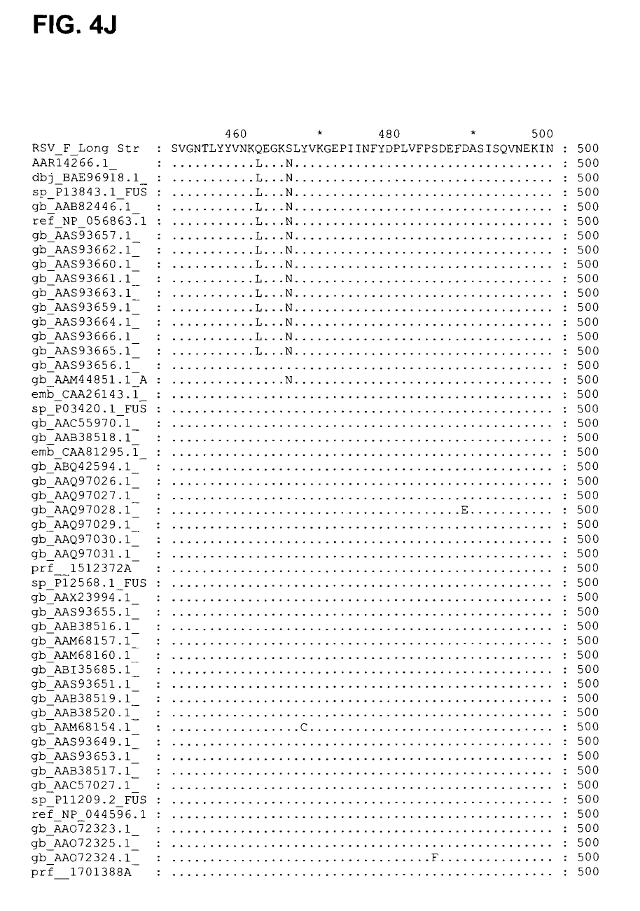

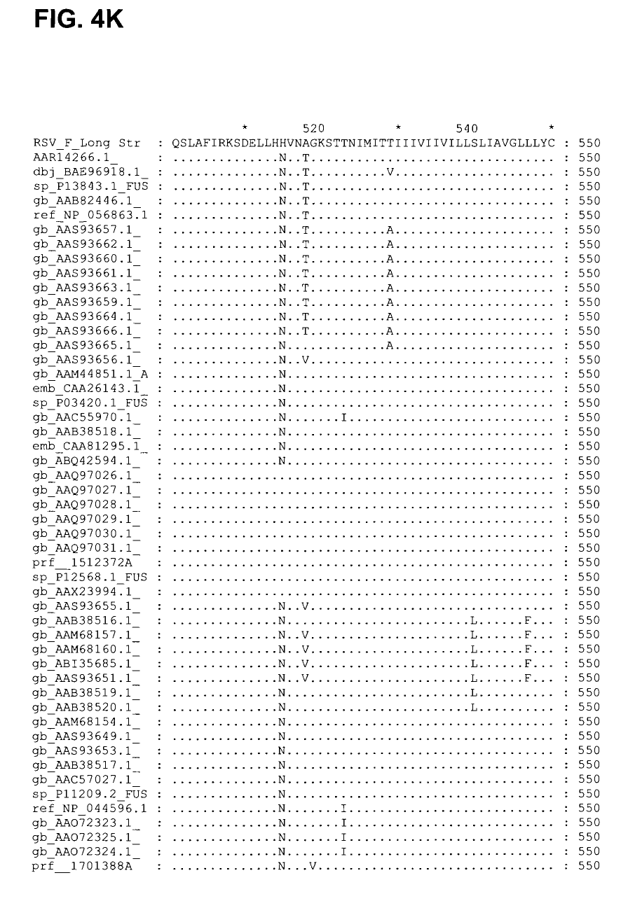

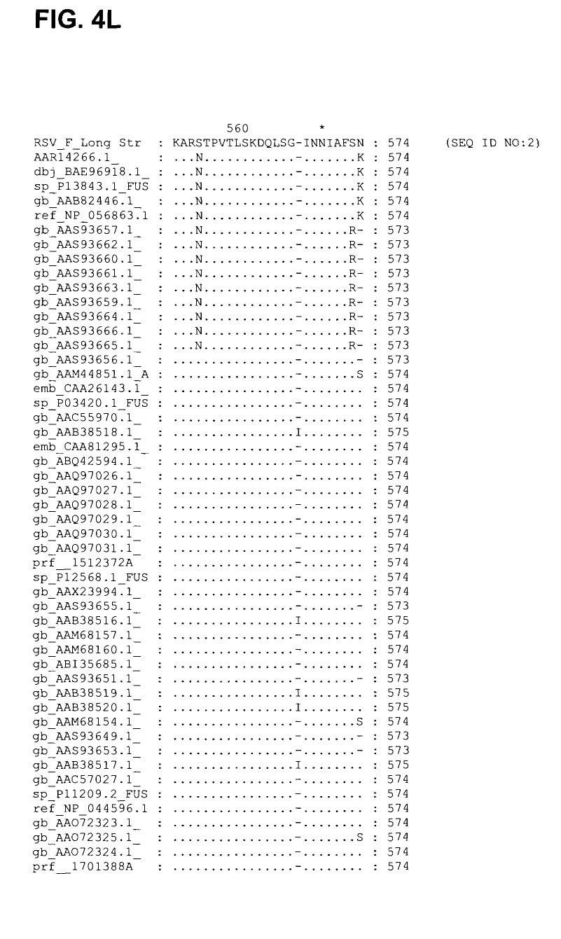

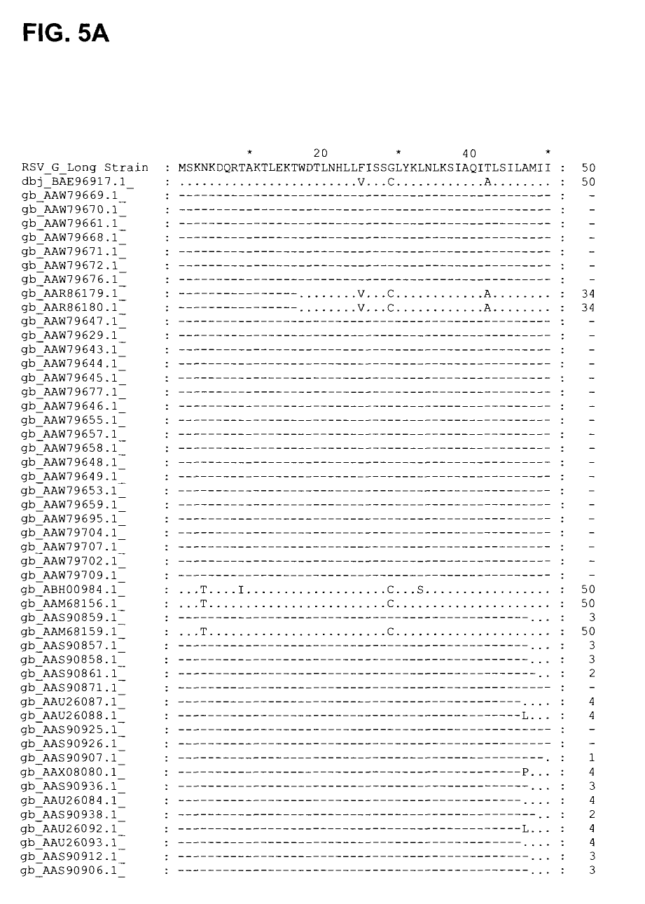

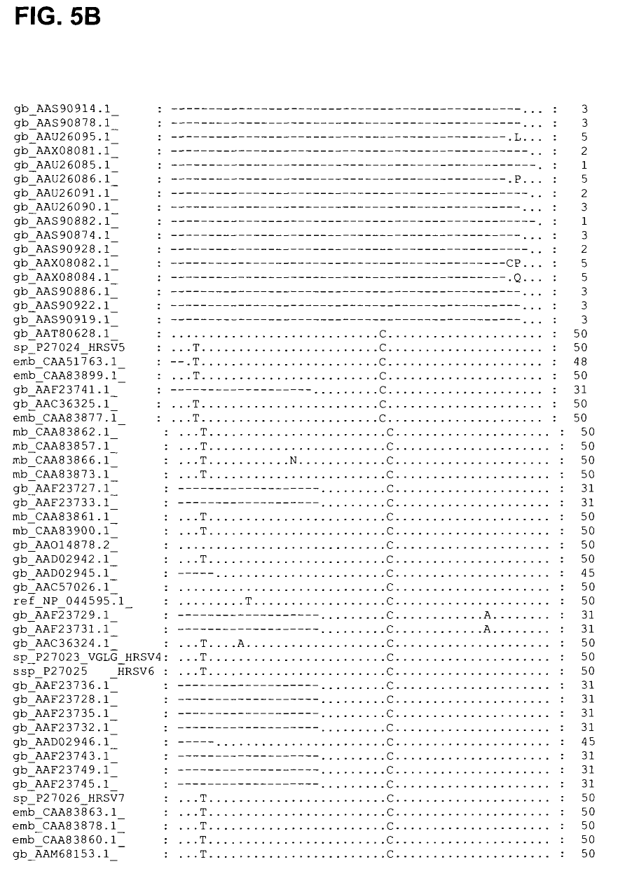

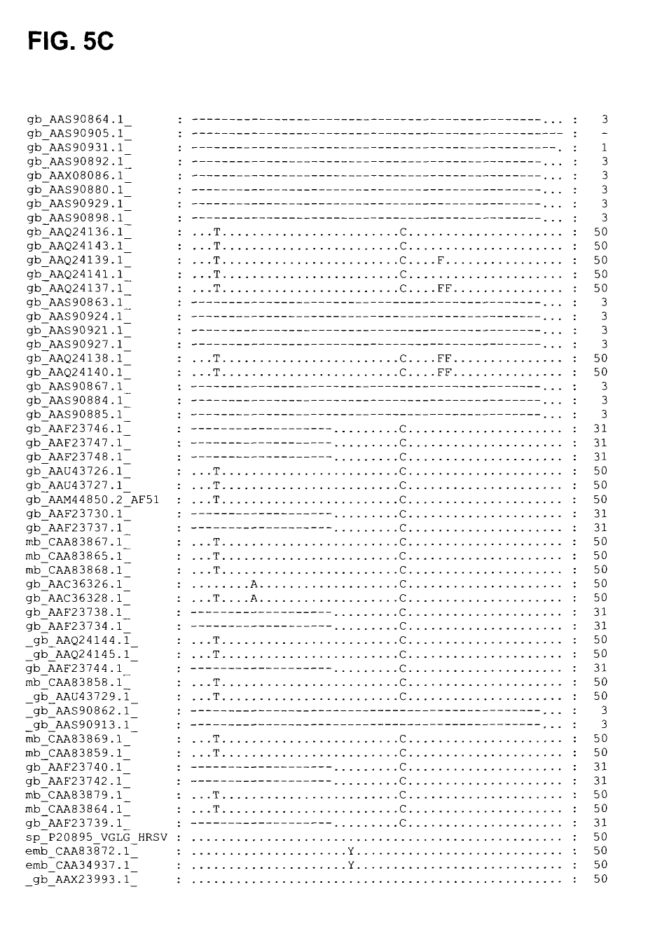

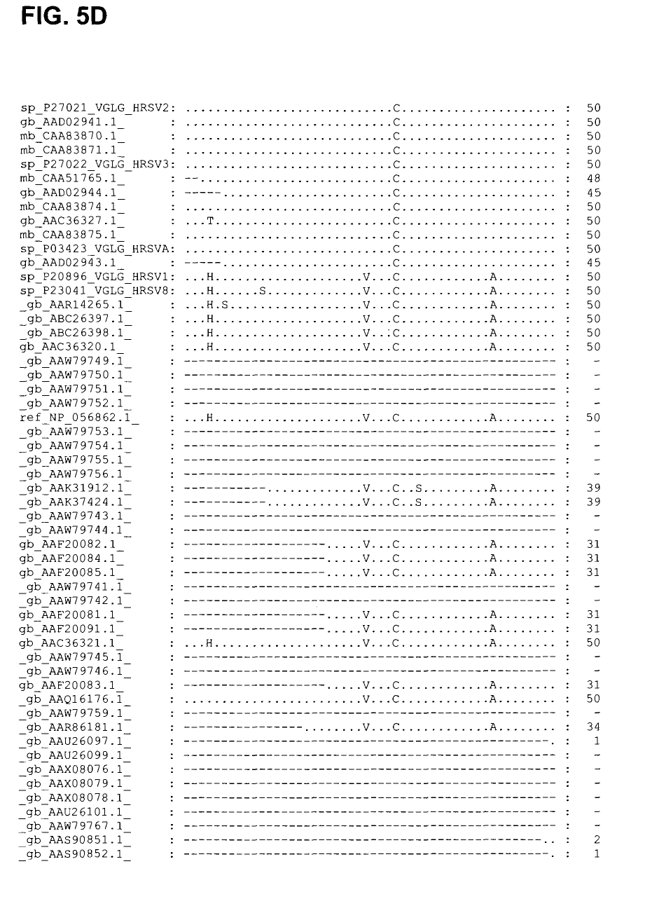

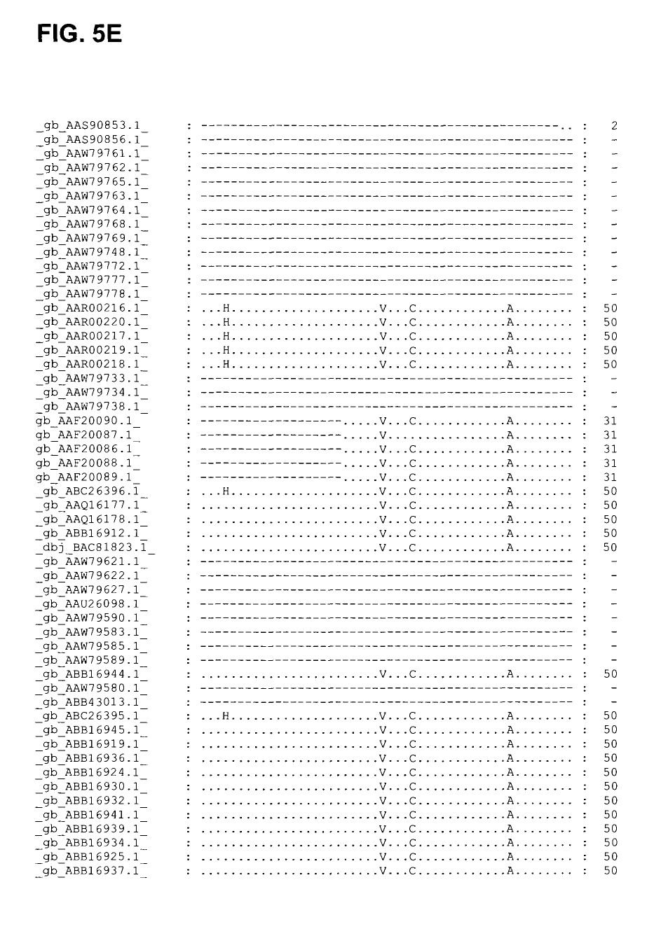

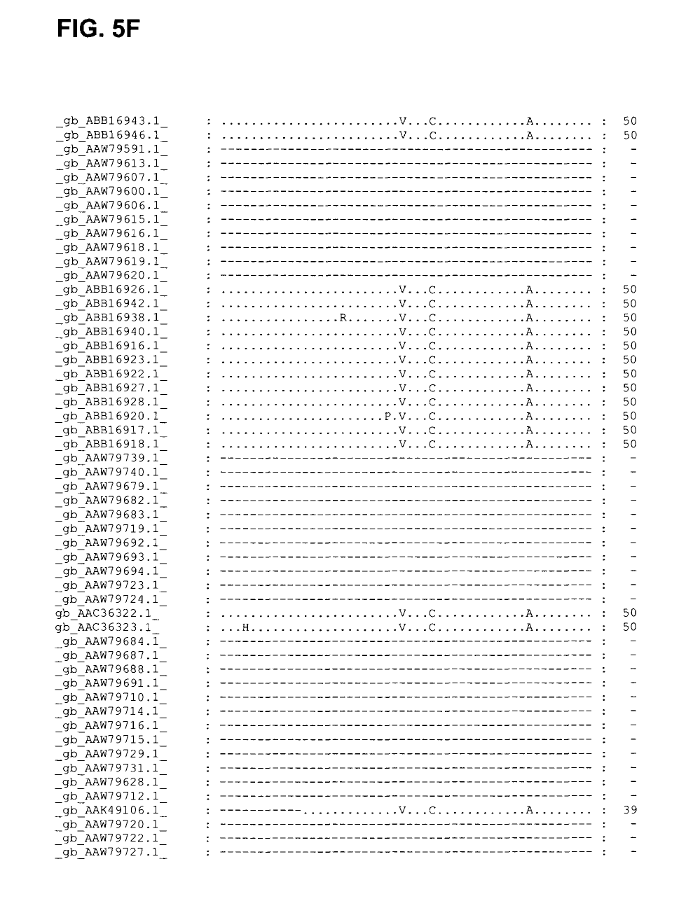

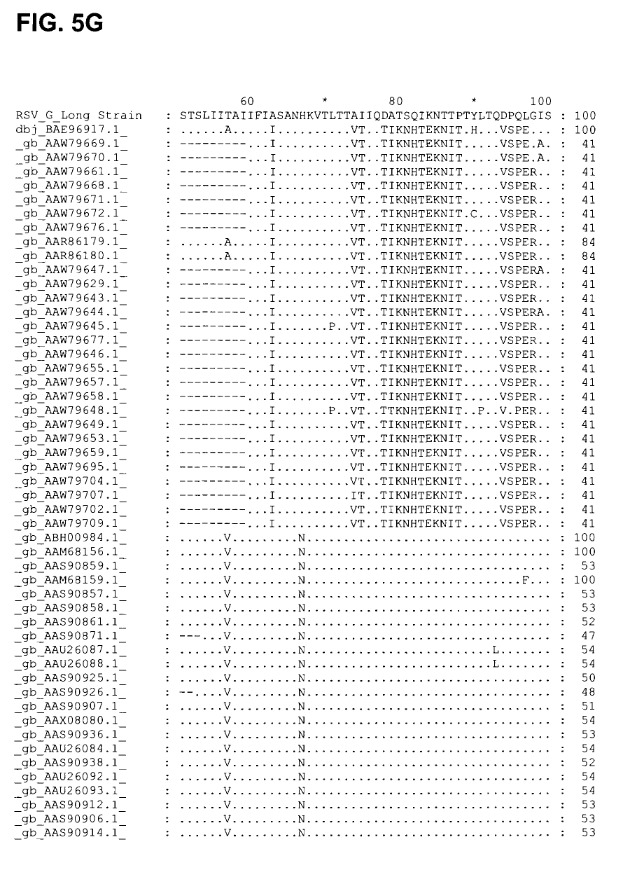

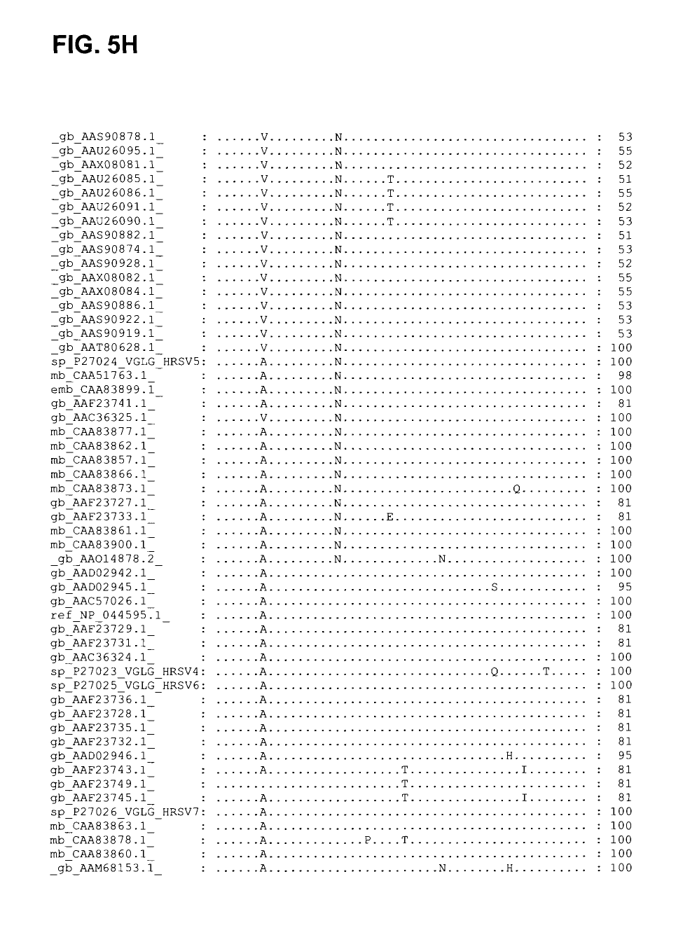

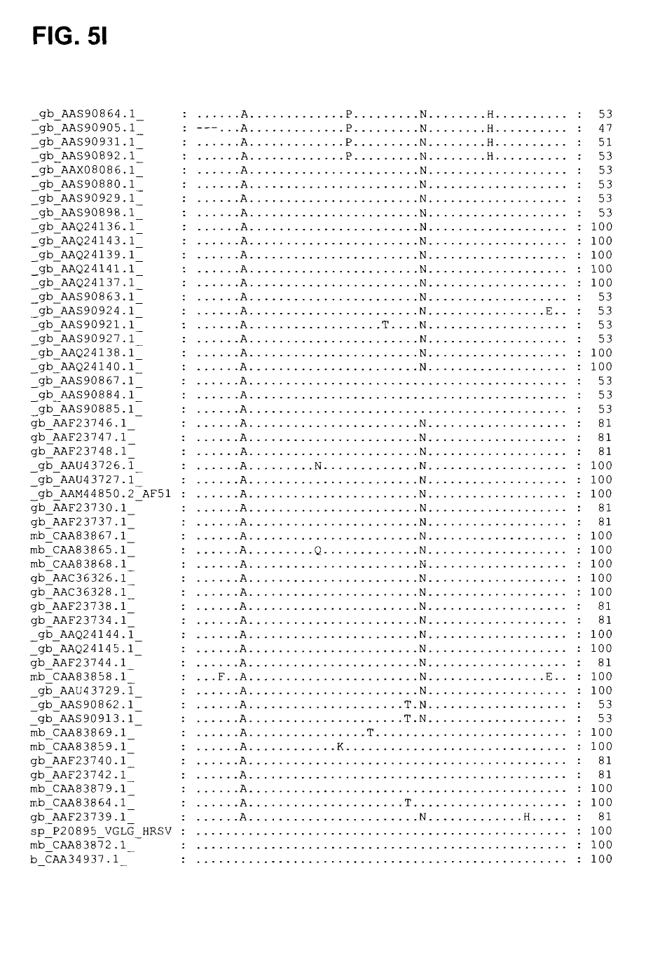

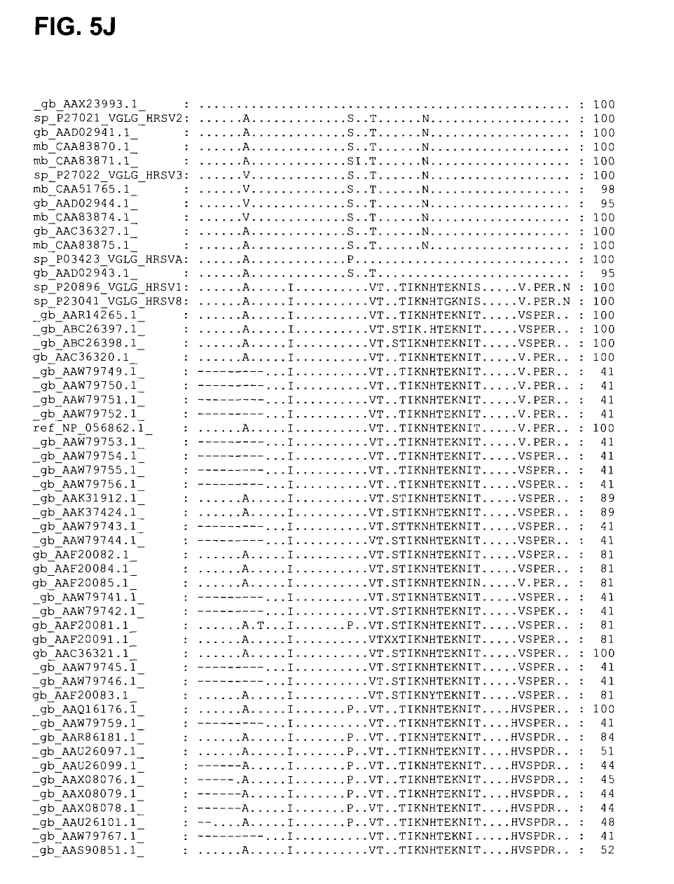

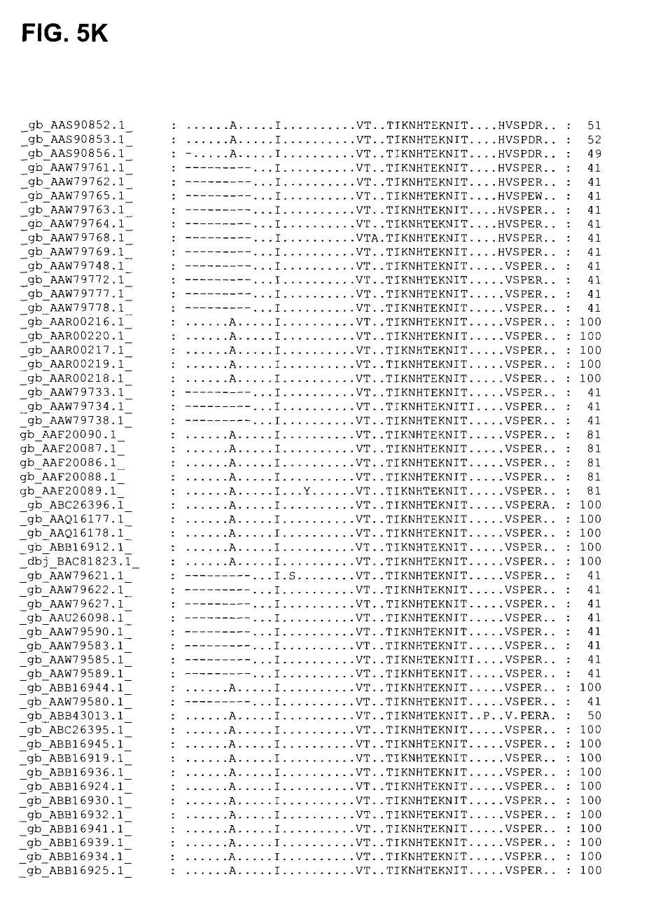

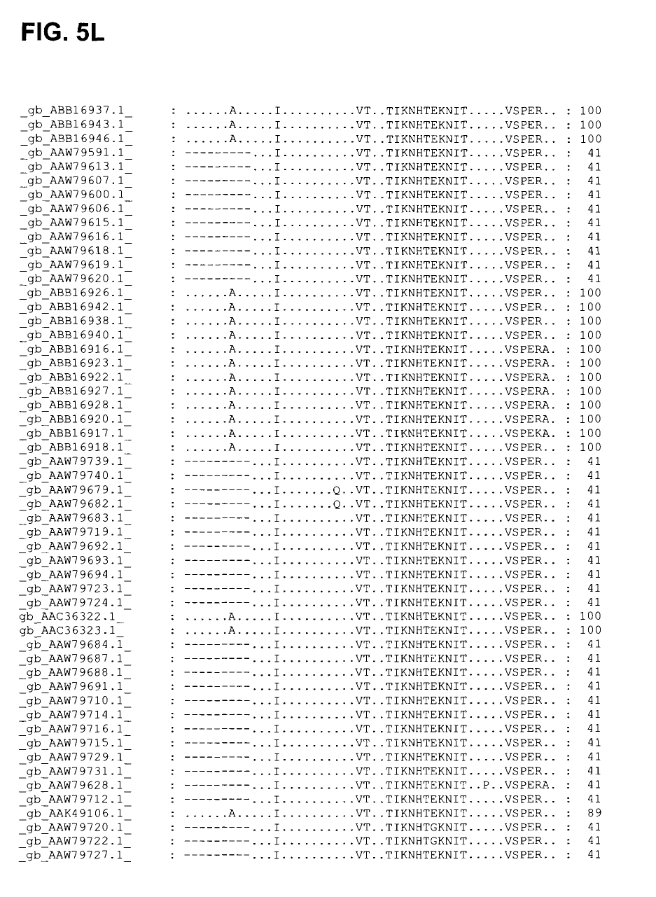

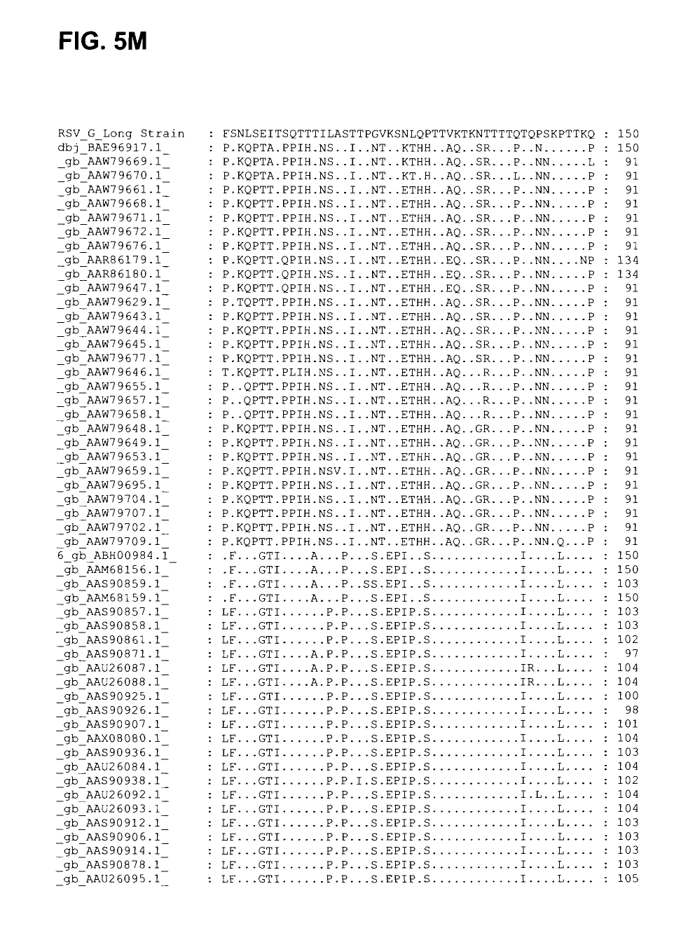

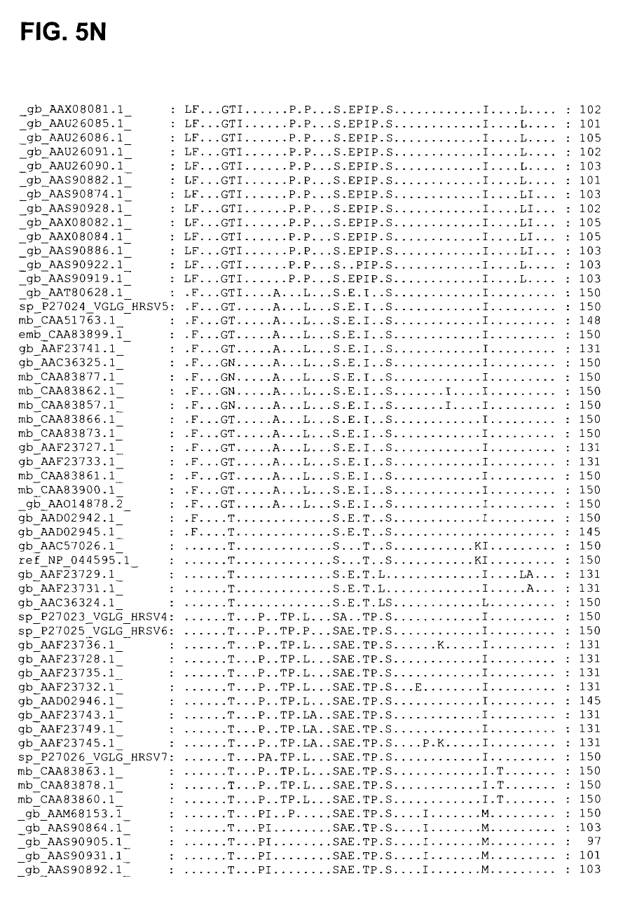

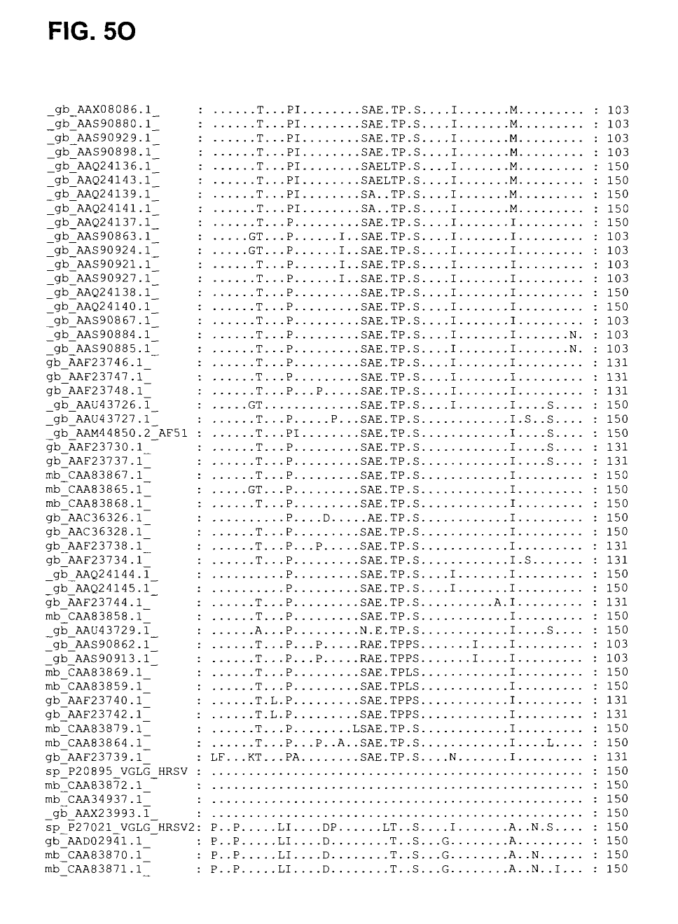

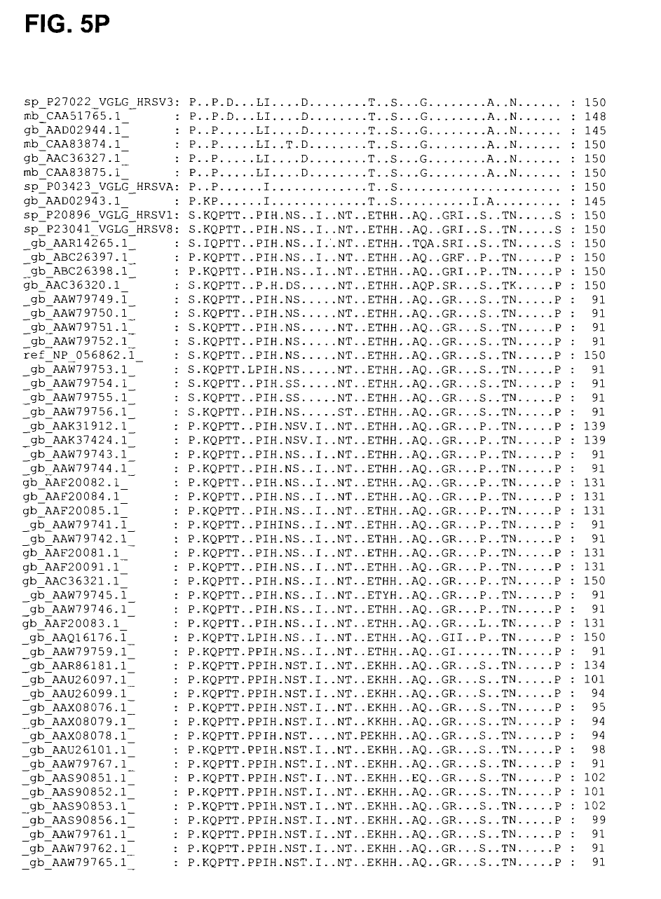

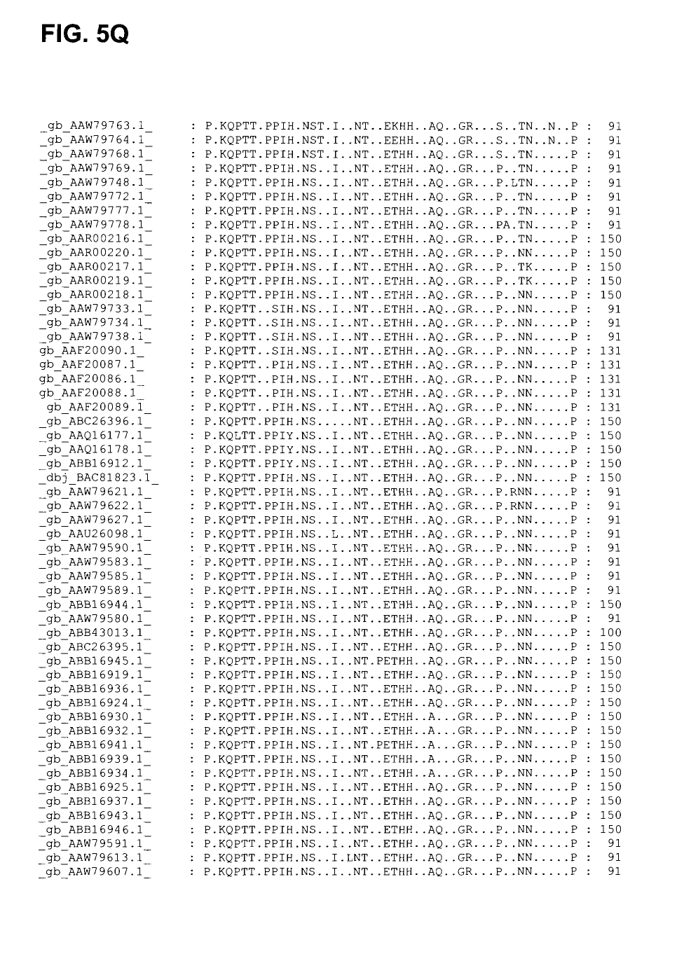

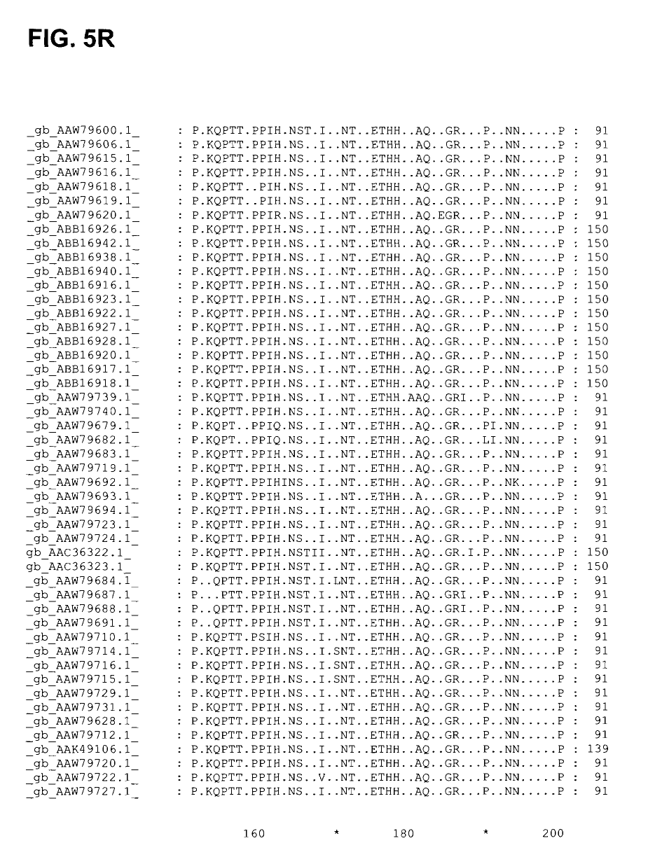

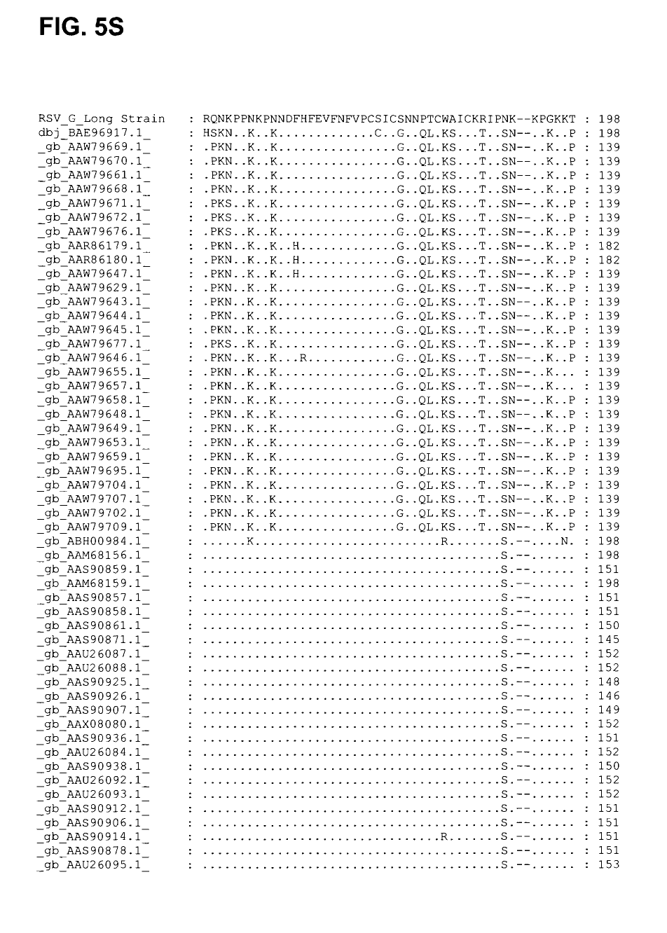

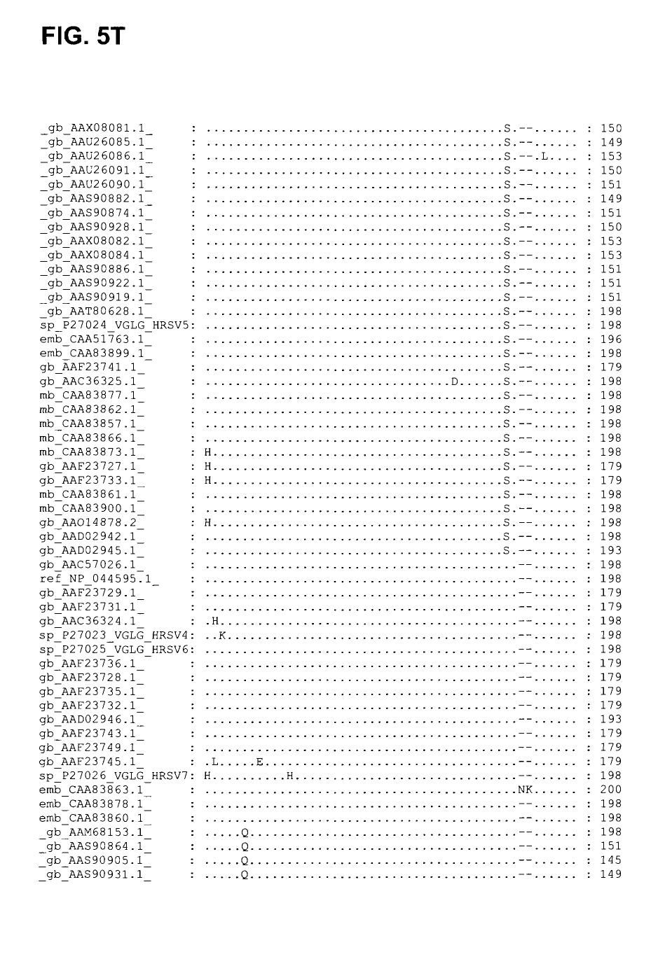

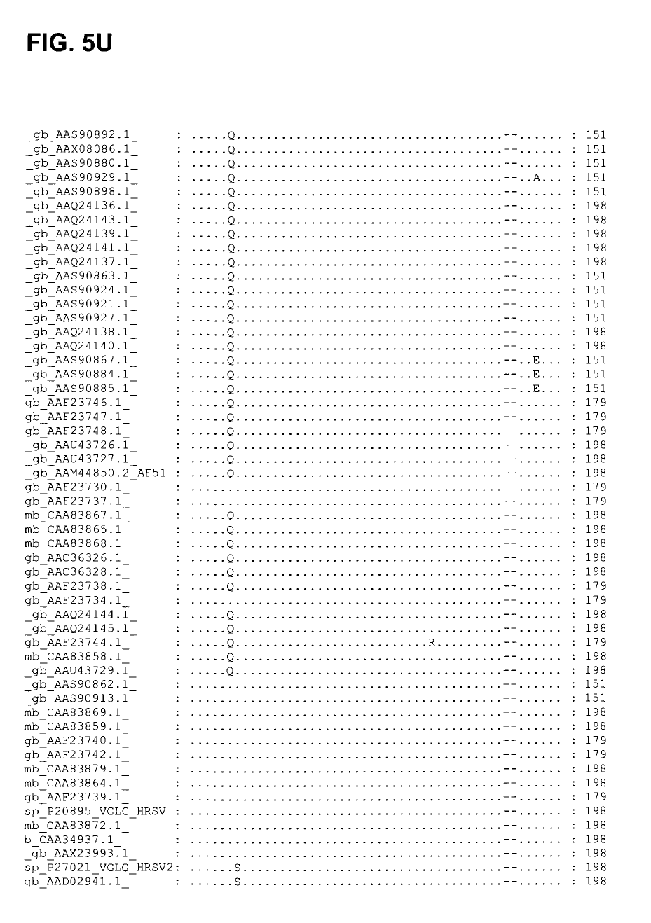

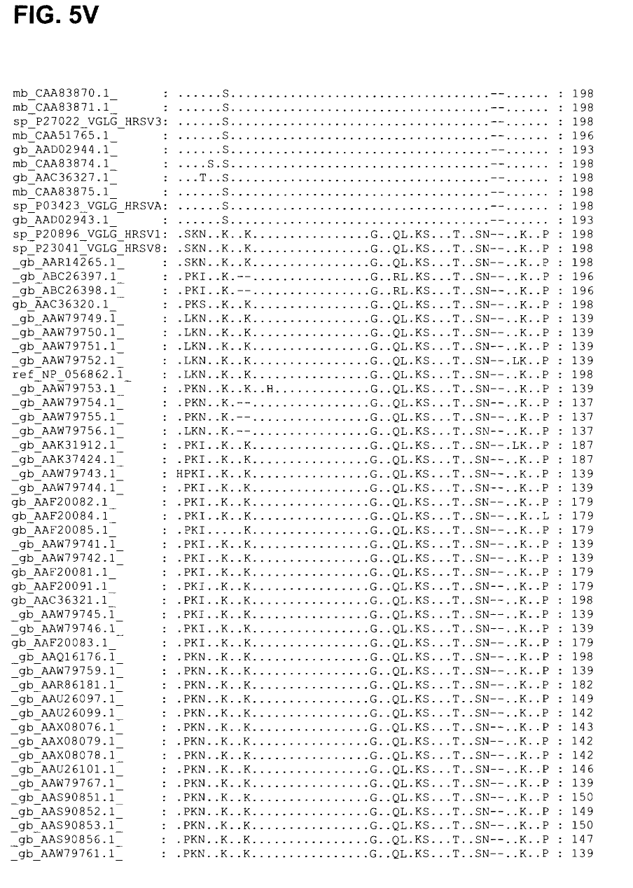

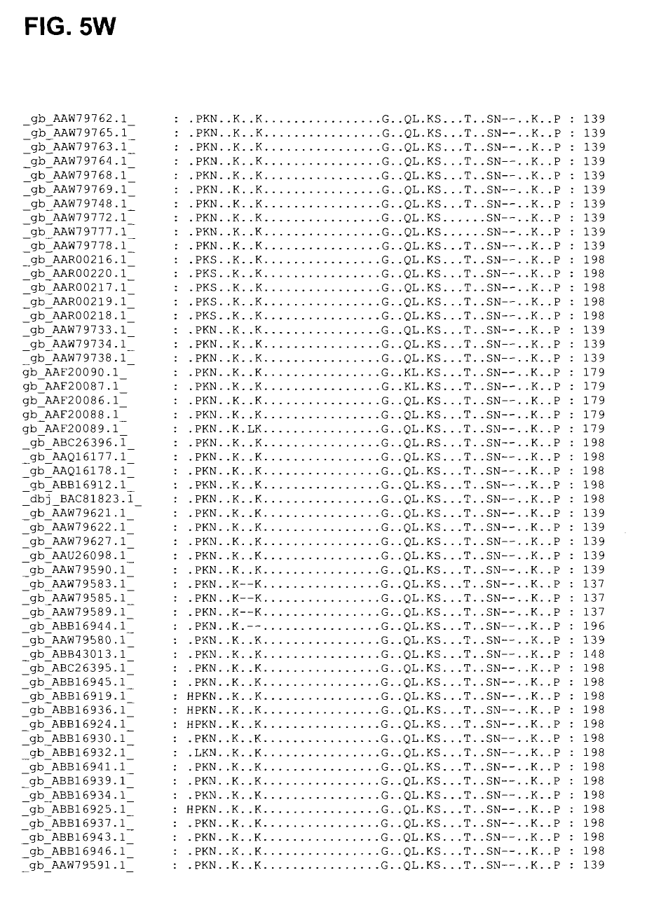

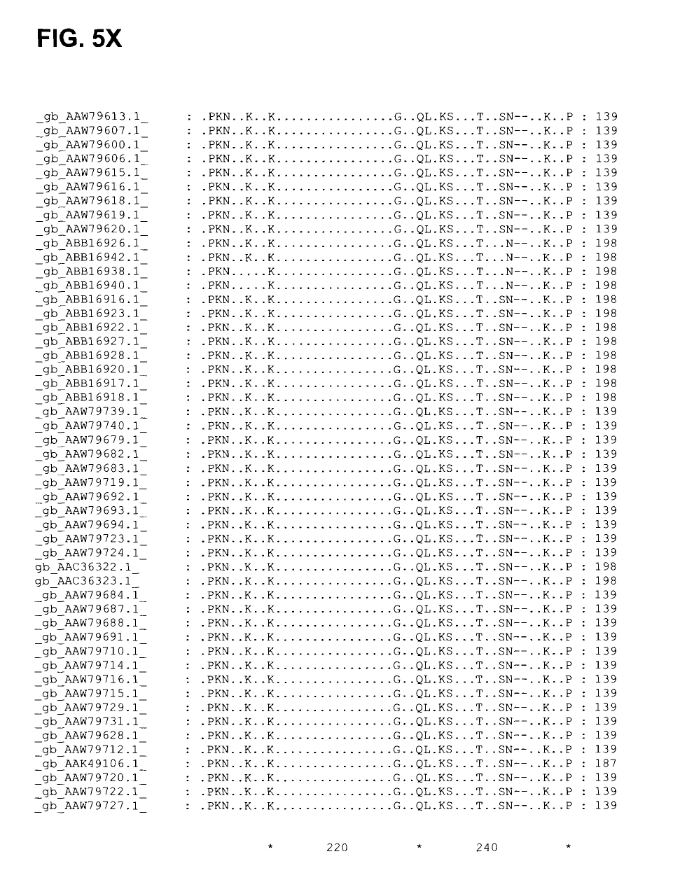

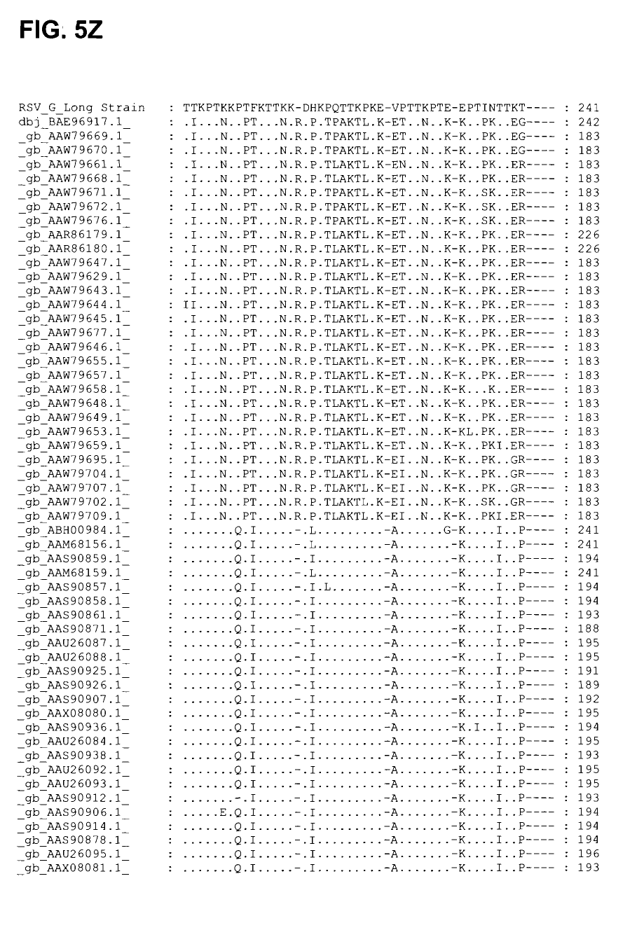

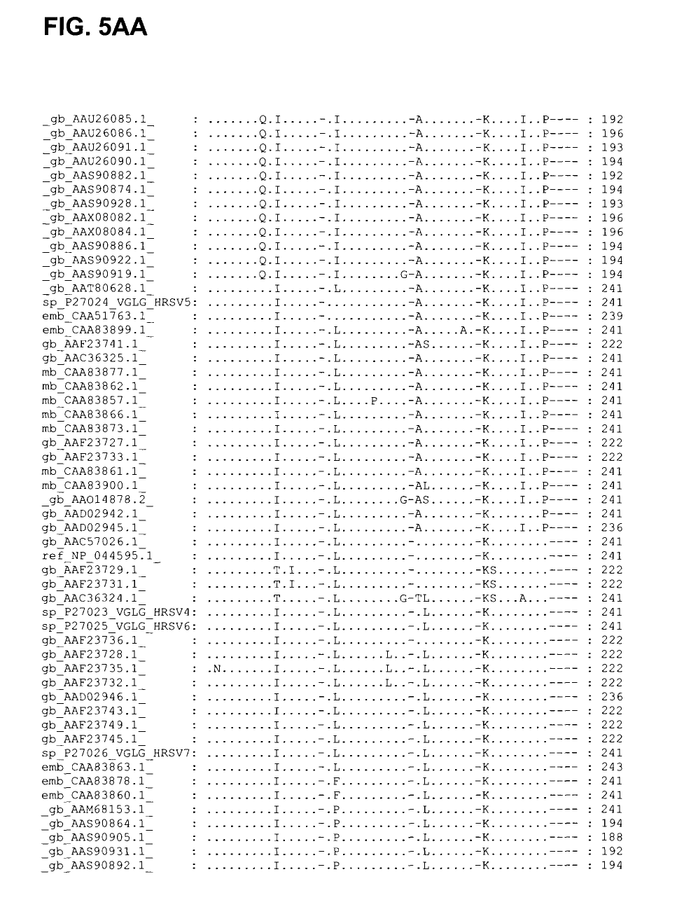

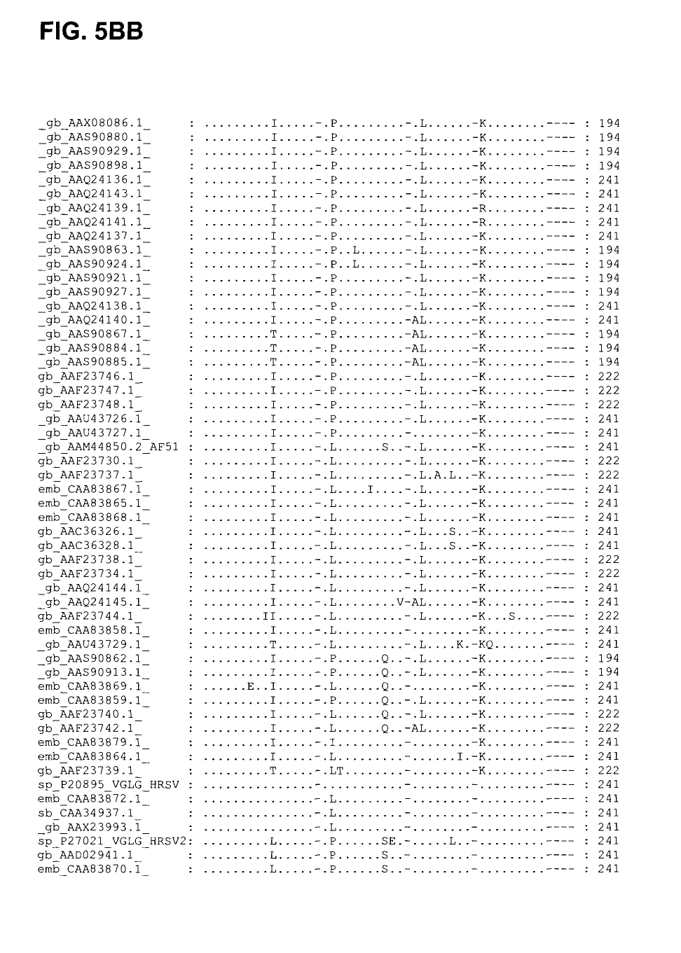

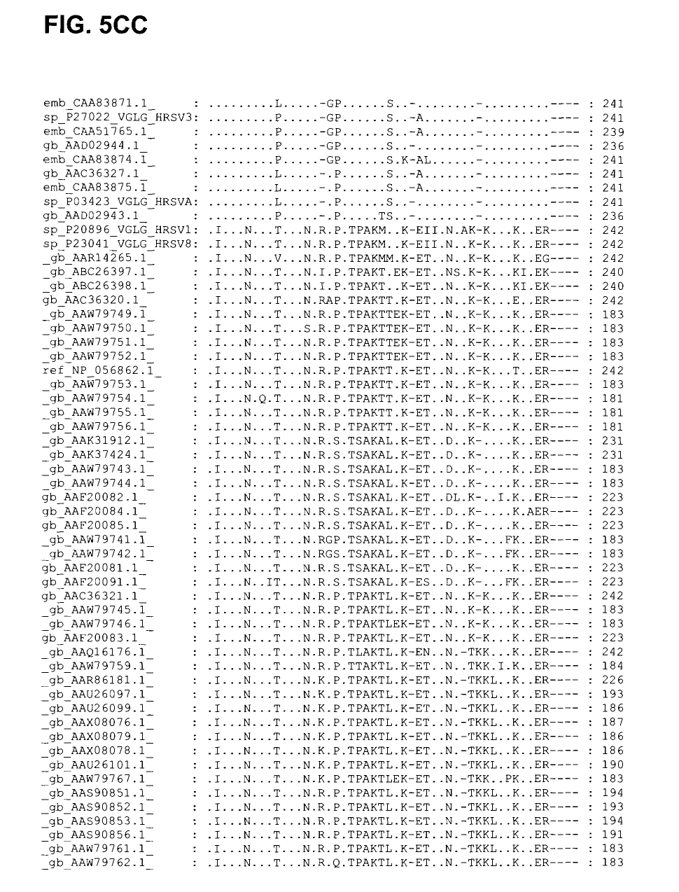

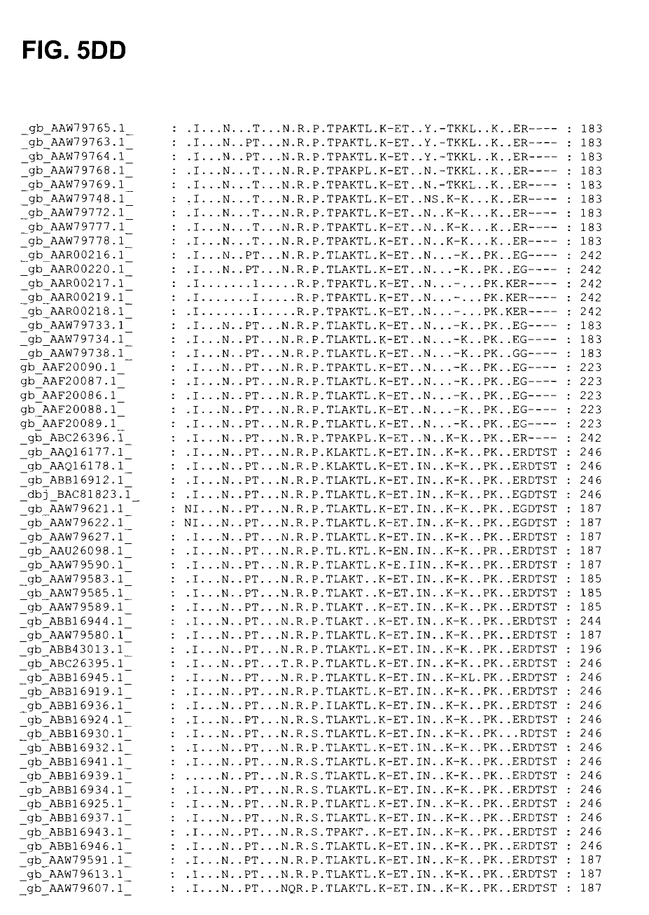

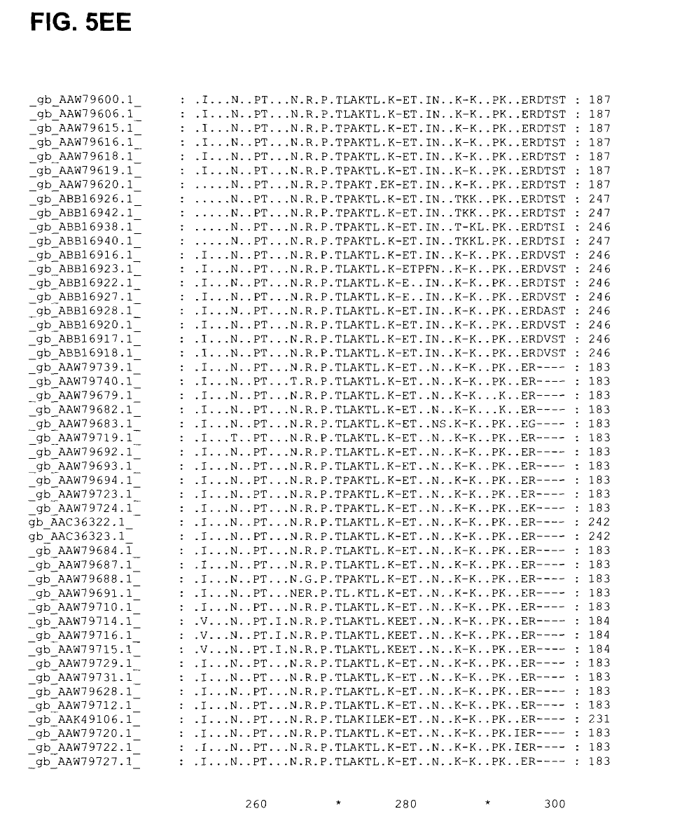

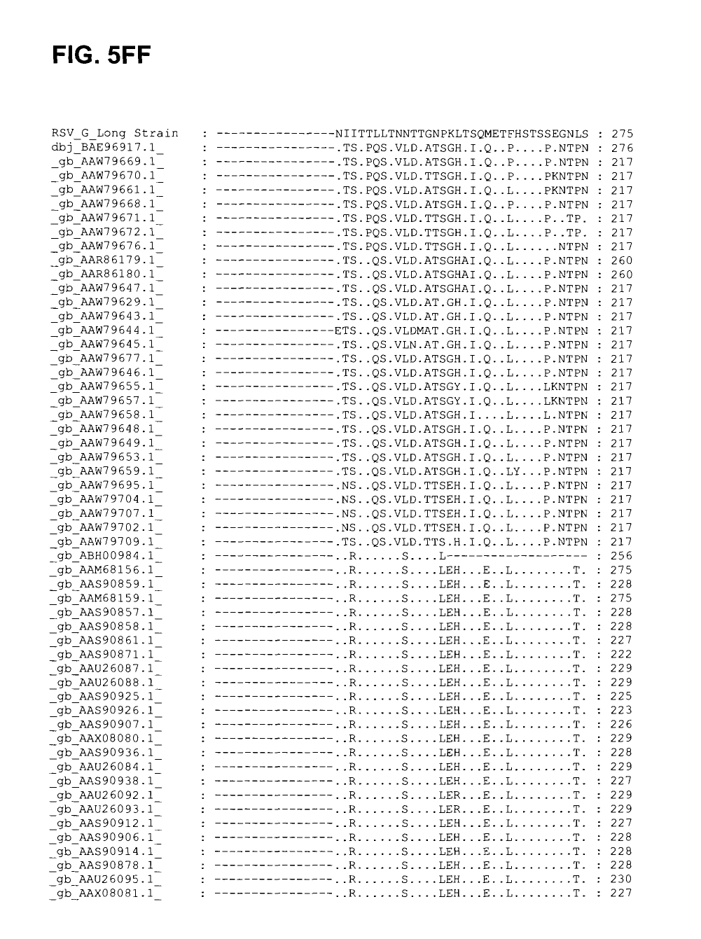

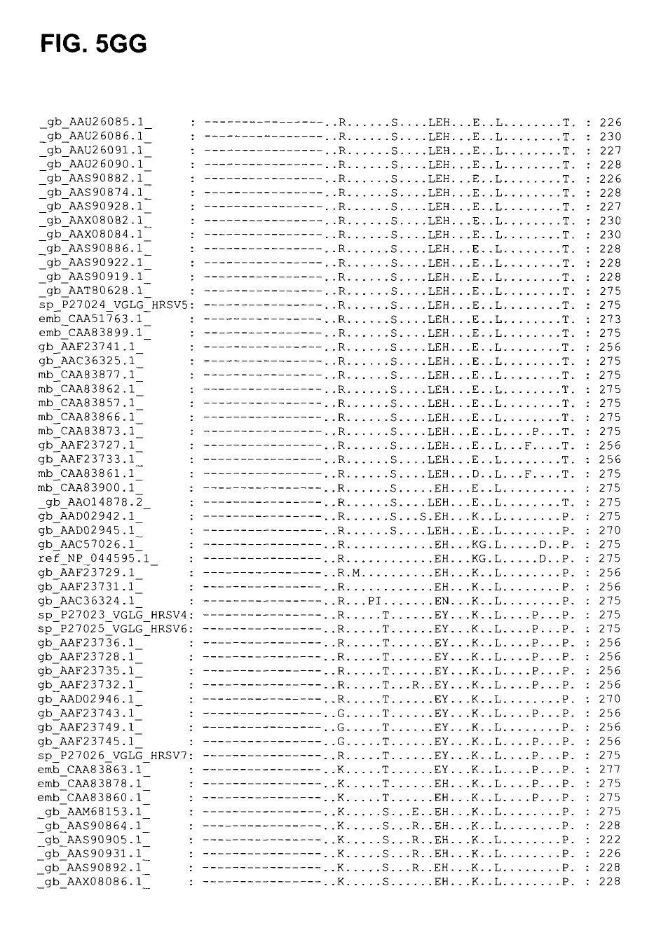

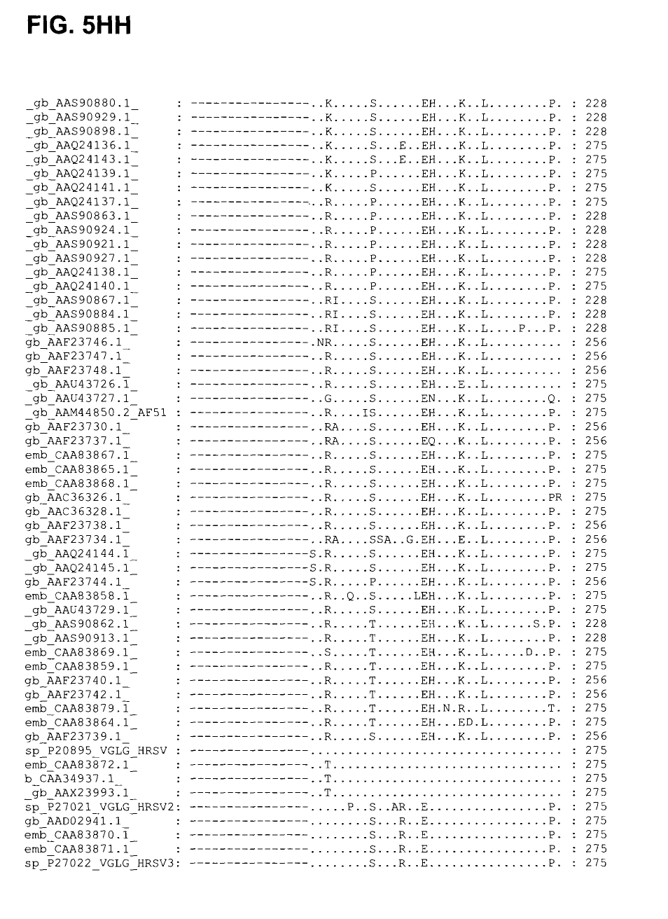

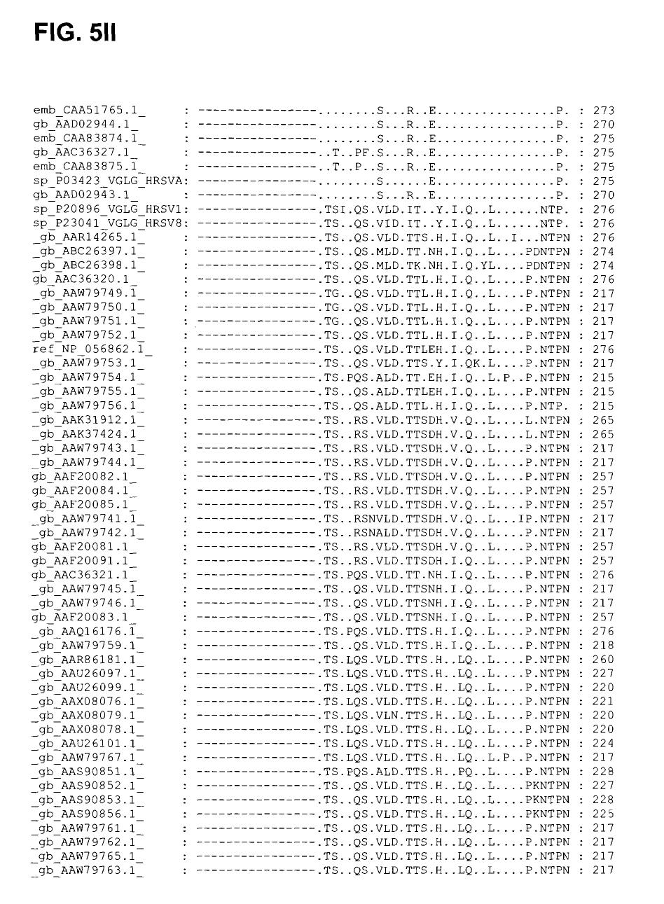

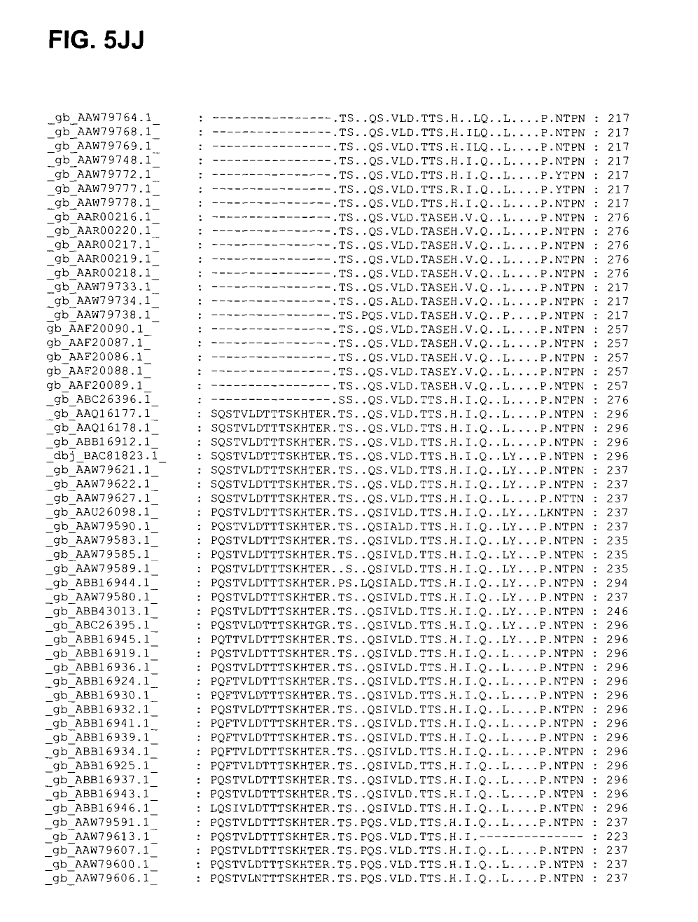

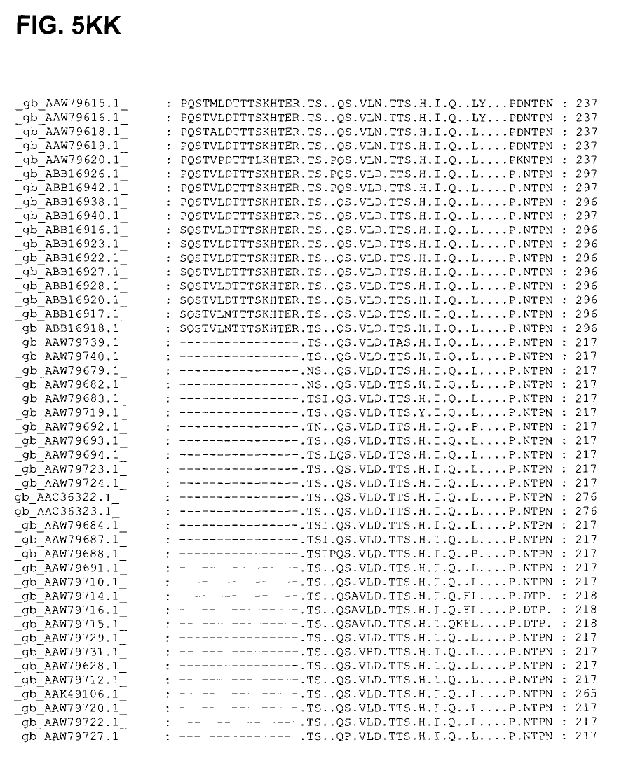

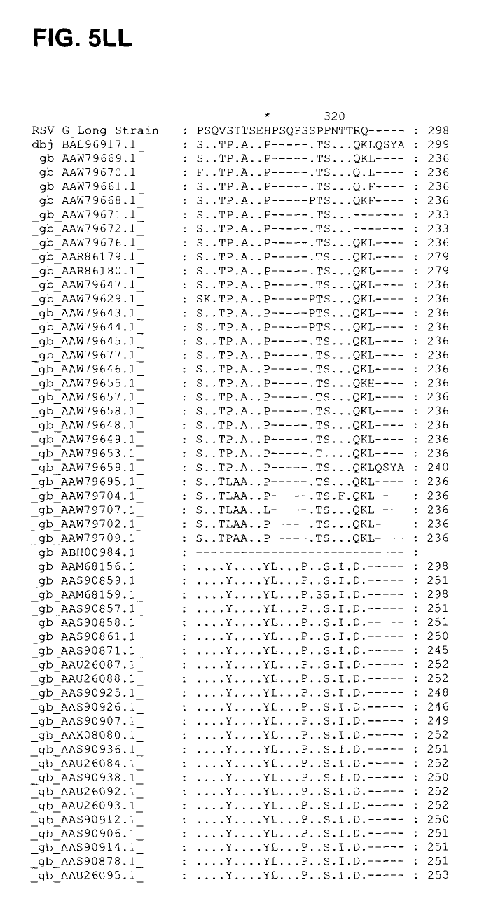

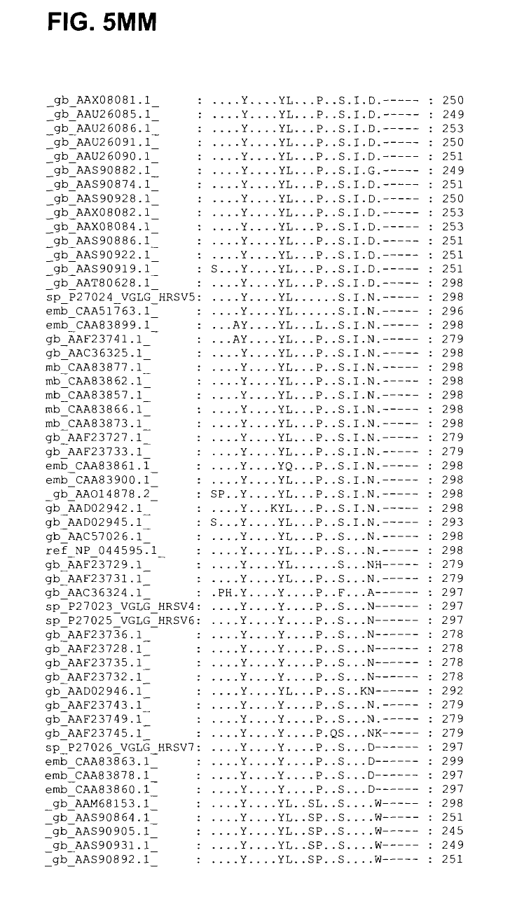

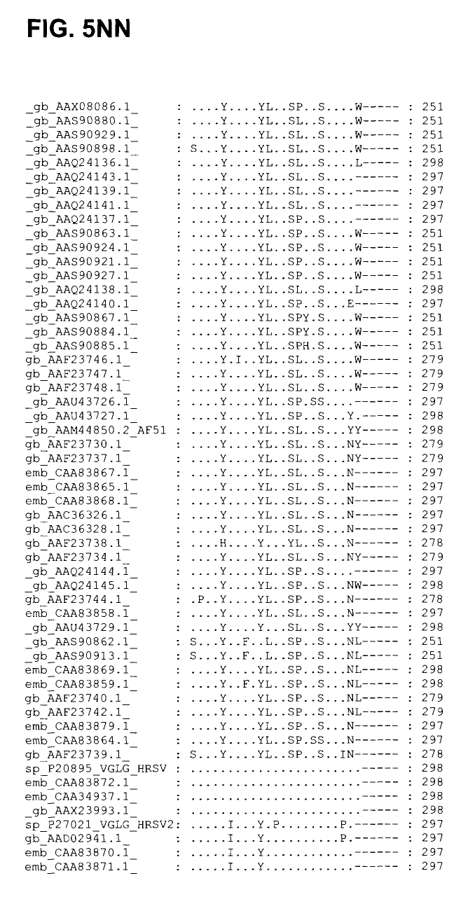

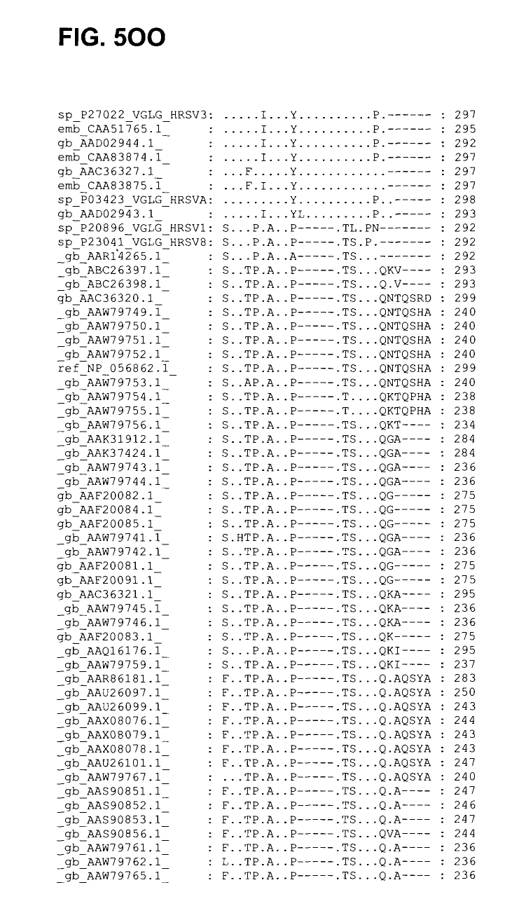

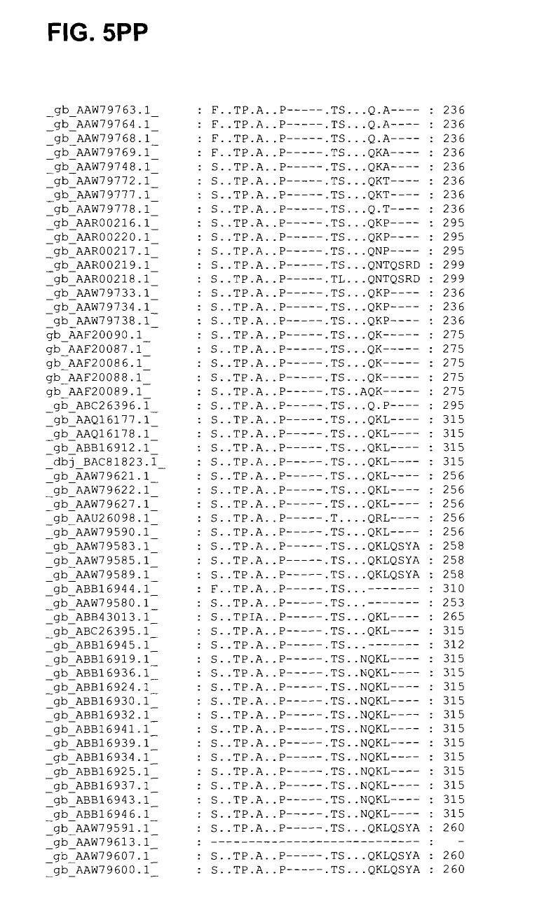

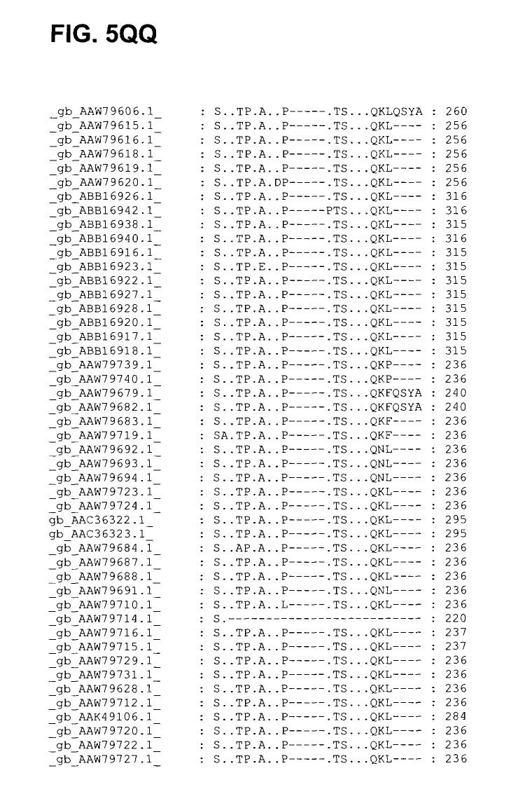

In one aspect, the disclosure relates to a respiratory syncytial virus (RSV) antigen including a chimeric polypeptide comprising in an N terminal to C terminal direction: a first F protein polypeptide domain; a G protein polypeptide domain; and a second F protein polypeptide domain. Such chimeric antigens are designated herein F2GF1 chimeric RSV antigens. The first F protein polypeptide domain can include at least an amino acid subsequence of the F2 (or F₂) subunit (or domain) produced in vivo by furin cleavage, for example, an amino acid sequence from residues 24 to 107 of a native F protein polypeptide. The native F protein polypeptide can be selected from any F protein of an RSV A or RSV B strain. In certain exemplary embodiments, the F protein is selected from the RSV Long strain (represented by SEQ ID NO:2 ATCC catalog #VR-26, GenBank #AY911262). To facilitate understanding of this disclosure, all amino acid residue positions are given with reference to (that is, the amino acid residue position corresponds to) the amino acid position of the RSV Long strain, although a comparable amino acids can be used from any RSV A or B strain. Comparable amino acid positions of any other RSV A or B strain can be determined easily by those of ordinary skill in the art by aligning the amino acid sequences of the selected RSV strain with that of Long strain using readily available and well-known alignment algorithms (such as BLAST, e.g., using default parameters, as shown in FIGS. 4 and 5). Additionally, the first F protein polypeptide domain can also include all or part of the amino acid sequence of “pep27” (for example, including all or a portion of amino acid residues 110 to 130 of a native F protein polypeptide). Additionally, or alternatively, the first F protein polypeptide domain can include signal peptide. Such a signal peptide can be the native F0 signal peptide (e.g., amino acids 1-23 of the F0 polypeptide), or it can be a heterologous signal peptide, for example selected based on the expression system of choice. In one exemplary embodiment, the F2 domain that includes a signal peptide includes amino acid residues 1-109 of a native F0 polypeptide.

Optionally, the first F protein polypeptide domain of the chimeric RSV antigen includes one or more amino acid modifications relative to a naturally occurring RSV F protein polypeptide. For example, such an amino acid modification can improve (e.g., increase) the solubility and/or stability of the chimeric RSV antigen. Such a modification can be a substitution of one or more amino acids, a deletion of one or more amino acids or an addition of one or more amino acids. In one example, the chimeric RSV antigen includes a first F protein polypeptide domain that has an amino acid other than methionine (such as an isoleucine) at position 79 (as compared to the native F0 polypeptide). This exemplary chimeric RSV antigen has been engineered to eliminate a secondary start site within the first F protein polypeptide domain. In another example, the amino acid modification includes an amino acid deletion or substitution that eliminates a furin cleavage site present in a naturally occurring RSV F protein. For example, the exemplary chimeric RSV antigen can be modified to eliminate a naturally occurring furin cleavage site that separates subunit F2 from pep27, e.g., by removal (either by deletion and/or substitution) of all or part of the furin cleavage site at positions 106-109.

The second F protein polypeptide domain typically includes all or part of the amino acid sequence of the F1 (or F₁) subunit (or domain) produced in vivo by furin cleavage. For example, the second F protein polypeptide domain can include all or part of an amino acid sequence from 161 to 524 of a native F protein polypeptide (e.g., from amino acid 151 to amino acid 524 of a native F protein). Optionally, the second F protein polypeptide domain comprises at least one amino acid modification that improves (e.g., increases) solubility and/or stability of the chimeric RSV antigen.

Located between the first F protein polypeptide domain, and the second F protein polypeptide domain in the chimeric RSV antigen is a G protein polypeptide domain. The intervening G protein polypeptide domain can include all or part of a native G protein polypeptide, such as the Long strain G protein represented by SEQ ID NO:4. In one exemplary embodiment, the G protein polypeptide is a subsequence (or fragment) of a native G protein polypeptide that includes all or part of amino acid residues 151-229 (e.g., from 149 to 229) of a native G protein polypeptide. In another embodiment, the G protein polypeptide domain includes an amino acid sequence from residues 128 to 229 of a native G protein polypeptide.

In certain embodiments of the chimeric RSV antigen, the G protein domain has been modified to reduce or prevent vaccine enhanced viral disease when the RSV antigen is administered to a subject (such as a human subject). Such a chimeric RSV antigen favorably includes a substitution of asparagine by alanine at position 191 (N191A) of the G protein.

In certain embodiments, at least one, sometimes two, and in some cases all three of the first F protein polypeptide domain, the G protein polypeptide domain, and/or the second F protein polypeptide domain correspond in sequence to the RSV A Long strain. Alternatively, one or more of the domains corresponds in sequence (or is derived from) another RSV A or B strain. Thus, the chimera can include F protein and G proteins amino acid sequences from one or more strain of RSV, such that the each of the two F protein components and the G protein component can be from the same strain, or from different strains. Where different strains are selected, the F protein and G protein components can each be from an A strain, or from a B strain, or from a combination of A and B strains.

In some instances, one or more of the polypeptide domains has one or more amino acid modification relative to the amino acid sequence of the naturally occurring strain from which it is derived. For example, the modification can be a substitution of one or more amino acids (such as two amino acids, three amino acids, four amino acids, five amino acids, up to about ten amino acids, or more). In certain embodiments, the RSV antigens can include one or more amino acid substitutions that replace a cysteine residue, such as a cysteine residue selected from amino acid residues 40, 72, 291, 392, 401, 412, 422, and/or 518 of the F2GF1 polypeptide (corresponding to residues 37, 69, 212, 313, 322, 333, 343 and 439 of the native F0 polypeptide. Alternatively, one or more of the cysteines can be replaced by a hydrophobic residue, such as leucine, isoleucine or valine. Additionally or alternatively, the chimeric RSV antigen can include one or more amino acid substitutions that replace a hydrophobic amino acid, such as a hydrophobic amino acid selected from positions 36 to 41 and/or positions 400 to 401, corresponding to residues 33-39 and 321-322 of F0.

Alternatively or additionally, the modification can include a deletion of one or more amino acids and/or an addition of one or more amino acids. Indeed, if desired, one or more of the polypeptide domains can be a synthetic polypeptide that does not correspond to any single strain, but includes component subsequences from multiple strains, or even from a consensus sequence deduced by aligning multiple strains of RSV virus polypeptides. In certain embodiments, one or more of the polypeptide domains is modified by the addition of an amino acid sequence that constitutes a tag, which facilitates subsequent processing or purification. Such a tag can be an antigenic or epitope tag, an enzymatic tag or a polyhistidine tag. Typically the tag is situated at one or the other end of the chimeric protein, such as at the C-terminus or N-terminus of the chimeric antigen or fusion protein.

Exemplary RSV antigens are represented by the amino acid sequences of SEQ ID NOs: 6, 8, 10, 12, 14, 16, 18, and 20. Nucleotide sequences encoding these exemplary F2GF1 polypeptides are designated SEQ ID NOs: 5, 7, 9, 11, 13, 15, 17 and 19, respectively. Additional exemplary RSV antigens are represented by SEQ ID NOs:21-43, with exemplary eukaryotic F2GF1 polypeptides represented by SEQ ID NOs:68 and 70 (nucleotide sequences SEQ ID NOs:67 and 69).

When expressed, the chimeric RSV antigens fold into a conformation that closely resembles the assembly of a mature cleaved F protein. The G protein component is situated between the F2 and F1 polypeptide subunits, forming a loop in which the immunodominant G protein epitope is located on the outside of the folded protein. In certain embodiments, the RSV antigen is a multimer of chimeric polypeptides. For example, the RSV antigen can favorably assemble into a trimer of F2GF1 chimeric RSV polypeptides, or into a higher order assembly or complex of multimers.

Another feature of this disclosure concerns immunogenic compositions that contain or include a F2GF1 chimeric RSV antigen in combination with a pharmaceutically acceptable carrier or excipient. Pharmaceutically acceptable carriers and excipients are well known and can be selected by those of skill in the art. For example, the carrier or excipient can favorably include a buffer. Optionally, the carrier or excipient also contains at least one component that stabilizes solubility and/or stability. Examples of solubilizing/stabilizing agents include detergents, for example, laurel sarcosine and/or tween. Alternative solubilizing/stabilizing agents include arginine, and glass forming polyols (such as sucrose, trehalose and the like).

Optionally, the immunogenic compositions also include an adjuvant. In the context of an immunogenic composition suitable for administration to a subject for the purpose of eliciting a protective immune response against RSV, the immunogenic composition (combination of antigen and adjuvant) is selected to elicit a Th1-type immune response.

The adjuvant is selected to be safe and minimally reactogenic in the subject, or population of subjects, to whom the immunogenic composition is administered. In the context of immunogenic compositions containing chimeric F2GF1 polypeptide antigens, to be safe, the adjuvant when administered in combination with the antigen, does not result in an immunopathological response, such as exacerbated RSV disease associated with a Th2-type immune response, in the subject. When the immunogenic composition is to be administered to a subject of a particular age group susceptible to (or at increased risk of) RSV infection, the adjuvant is selected to be safe and effective in the subject or population of subjects. Thus, when formulating an immunogenic composition containing a chimeric RSV antigen for administration in an elderly subject (such as a subject greater than 65 years of age), the adjuvant is selected to be safe and effective in elderly subjects. Similarly, when the immunogenic composition containing the chimeric RSV antigen is intended for administration in neonatal or infant subjects (such as subjects between birth and the age of two years), the adjuvant is selected to be safe and effective in neonates and infants.

Additionally, the adjuvant is typically selected to enhance a protective immune response when administered via a route of administration, by which the immunogenic composition is administered. For example, when formulating an immunogenic composition containing a chimeric RSV antigen for nasal administration, proteosome and protollin are favorable Th1 biasing adjuvants. In contrast, when the immunogenic composition is formulated for intramuscular administration, adjuvants including one or more of 3D-MPL, squalene (e.g., QS21), liposomes, and/or oil and water emulsions are favorably selected.

In certain exemplary embodiment, the immunogenic composition containing the chimeric RSV antigen is formulated for intramuscular injection in pharmaceutically acceptable excipient containing a buffer and an adjuvant that includes 3D-MPL, optionally with alum or with QS21, e.g. in a liposomal formulation, at a concentration suitable for administration to neonates. In another embodiment, the chimeric RSV antigen is formulated in an oil-in-water emulsion (e.g., with or without 3D-MPL) In another exemplary embodiment, the immunogenic composition containing the chimeric RSV antigen is similarly formulated with a concentration of adjuvant that enhances an immune response in an elderly subject. In another exemplary embodiment, the immunogenic composition containing the chimeric RSV antigen is formulated for intranasal administration with a proteosome or protollin adjuvant.

In certain embodiments, the immunogenic compositions are administered (e.g., prophylactically) to reduce or prevent infection with RSV. In some embodiments, the immunogenic compositions are administered prophylactically to reduce or prevent a pathological response following infection with RSV. Optionally, the immunogenic compositions containing a chimeric RSV antigen are formulated with at least one additional antigen of a pathogenic organism other than RSV. For example, the pathogenic organism can be a pathogen of the respiratory tract (such as a virus or bacterium that causes a respiratory infection). In certain cases, the immunogenic composition contains an antigen derived from a pathogenic virus other than RSV, such as a virus that causes an infection of the respiratory tract, such as influenza or parainfluenza. In other embodiments, the additional antigens are selected to facilitate administration or reduce the number of inoculations required to protect a subject against a plurality of infectious organisms. For example, the antigen can be derived from any one or more of hepatitis B, diphtheria, tetanus, pertussis, Hemophilus influenza, poliovirus, or Pneumococcus, among others.

Another aspect of this disclosure concerns recombinant nucleic acids that encode chimeric RSV antigens as described above. In certain embodiments, the recombinant nucleic acids are codon optimized for expression in a selected prokaryotic or eukaryotic host cell. To facilitate replication and expression, the nucleic acids can be incorporated into a vector, such as a prokaryotic or a eukaryotic expression vector. Host cells including recombinant F2GF1 chimeric RSV antigen-encoding nucleic acids are also a feature of this disclosure. Favorable host cells include prokaryotic (i.e., bacterial) host cells, such as E. coli, as well as numerous eukaryotic host cells, including fungal (e.g., yeast) cells, insect cells, plant cells, and mammalian cells (such as CHO cells).

Accordingly, the use of the chimeric RSV F2GF1 polypeptides, and nucleic acids that encode them, in the preparation of a medicament (for example, an immunogenic composition) for treating (either therapeutically following or prophylactically prior to) exposure to or infection by RSV is also a feature of this disclosure. Likewise, methods for eliciting an immune response against RSV in a subject are a feature of this disclosure. Such methods include administering an immunogenically effective amount of a composition comprising a F2GF1 chimeric RSV antigen to a subject, such as a human subject. Commonly, the composition includes an adjuvant that enhances the immune response. The composition is formulated to elicit an immune response specific for RSV without enhancing viral disease following contact with RSV. That is, the immunogenic composition is formulated to, and results in an immune response that reduces or prevents infection with a RSV and/or reduces or prevents a pathological response following infection with a RSV. Although the composition can be administered by a variety of different routes, most commonly, the immunogenic compositions are delivered by an intramuscular or intranasal route of administration.

### Terms

Unless otherwise explained, all technical and scientific terms used herein have the same meaning as commonly understood by one of ordinary skill in the art to which this disclosure belongs. Definitions of common terms in molecular biology can be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

The singular terms “a,” “an,” and “the” include plural referents unless context clearly indicates otherwise. Similarly, the word “or” is intended to include “and” unless the context clearly indicates otherwise. The term “plurality” refers to two or more. It is further to be understood that all base sizes or amino acid sizes, and all molecular weight or molecular mass values, given for nucleic acids or polypeptides are approximate, and are provided for description. Additionally, numerical limitations given with respect to concentrations or levels of a substance, such as an antigen, are intended to be approximate. Thus, where a concentration is indicated to be at least (for example) 200 pg, it is intended that the concentration be understood to be at least approximately (or “about” or “˜”) 200 pg.

Although methods and materials similar or equivalent to those described herein can be used in the practice or testing of this disclosure, suitable methods and materials are described below. The term “comprises” means “includes.” Thus, unless the context requires otherwise, the word “comprises,” and variations such as “comprise” and “comprising” will be understood to imply the inclusion of a stated compound or composition (e.g., nucleic acid, polypeptide, antigen) or step, or group of compounds or steps, but not to the exclusion of any other compounds, composition, steps, or groups thereof. The abbreviation, “e.g.” is derived from the Latin exempli gratia, and is used herein to indicate a non-limiting example. Thus, the abbreviation “e.g.” is synonymous with the term “for example.”

In order to facilitate review of the various embodiments of this disclosure, the following explanations of terms are provided. Additional terms and explanations can be provided in the context of this disclosure.

Respiratory syncytial virus (RSV) is a pathogenic virus of the family Paramyxoviridae, subfamily Pneumovirinae, genus Pneumovirus. The genome of RSV is a 15,222 nucleotide-long, single-stranded, negative-sense RNA molecule, which encodes 11 proteins. Tight association of the RNA genome with the viral N protein forms a nucleocapsid wrapped inside the viral envelope. Two groups of human RSV strains have been described, the A and B groups, based on differences in the antigenicity of the G glycoprotein. Numerous strains of RSV have been isolated to date. Exemplary strains are indicated by GenBank and/or EMBL Accession number in FIGS. 4 and 5. Additional strains of RSV are likely to be isolated, and are encompassed within the genus of RSV. Similarly, the genus of RSV encompasses variants arising from naturally occurring (e.g., previously or subsequently identified strains) by genetic drift, or artificial synthesis and/or recombination.

The term “F protein” or “Fusion protein” or “F protein polypeptide” or Fusion protein polypeptide” refers to a polypeptide or protein having all or part of an amino acid sequence of an RSV Fusion protein polypeptide. Similarly, the term “G protein” or “G protein polypeptide” refers to a polypeptide or protein having all or part of an amino acid sequence of an RSV Attachment protein polypeptide. Numerous RSV Fusion and Attachment proteins have been described and are known to those of skill in the art. FIGS. 4 and 5 set out exemplary F and G protein variants (for example, naturally occurring variants) publicly available as of the filing date of this disclosure.

A “chimeric F2GF1 polypeptide” or an “F2GF1 antigen” or “F2GF1 polypeptide antigen” is a chimeric polypeptide that incorporates polypeptide components, typically including antigenic determinants or epitopes of both an RSV F protein and an RSV G protein, and includes in an N-terminal to C-terminal orientation: at least one subsequence or fragment of an F2 subunit or domain (e.g., including all or part of amino acid residues 1-107 of a native F protein polypeptide, and optionally including a pep27 domain, for example amino acid residues 108-130 of F0); at least one subsequence of a G protein polypeptide; and at least one subsequence of an F1 subunit or domain (e.g., including all or part of amino acids 151-524 of a native F protein polypeptide). The term subunit and domain are used interchangeably in reference to structural domains of the F protein and/or F0 polypeptide. In vivo, proteolytic cleavage of the mature F0 polypeptide by a furin protease at two conserved furin consensus sequences, RAR/KR¹⁰⁹ (FCS-2) and KKRKRR¹³⁶ (FCS-1), resulting in the generation of three proteolytic fragments, the large membrane-anchored subunit F1 with a hydrophobic fusion peptide at its N terminus, the small subunit F2 which is linked to F1 via a disulfide bridge, and a small peptide composed of 27 amino acids (pep27) originally located between the two cleavage sites. It will be recognized by those of skill in the art that the abbreviations F0, F1 and F2 are commonly designated F₀, F₁ and F₂ in the scientific literature. The term chimeric in this context includes polypeptides in which the F and G protein components are both from the same serotype or strain, as well as polypeptides in which the individual F and G protein components are from different serotypes or strains.

A “variant” when referring to a nucleic acid or a protein (e.g., an RSV F or G protein or protein domain, or an F2GF1 chimeric polypeptide) is a nucleic acid or a polypeptide that differs from a reference nucleic acid or protein. Usually, the difference(s) between the variant and the reference nucleic acid or protein constitute a proportionally small number of differences as compared to the reference. Such differences can be amino acid additions, deletions or substitutions. Thus, a variant typically differs by no more than about 1%, or 2%, or 5%, or 10%, or 15%, or 20% of the nucleotide or amino acid residues. Thus, a variant in the context of an RSV F or G protein, or a chimeric F2GF1 polypeptide, typically shares at least 80%, or 85%, more commonly, at least about 90% or more, such as 95%, or even 98% or 99% sequence identity with a reference protein, e.g., the reference sequences illustrated in SEQ ID NO:2 and 4, or any of the exemplary F2GF1 polypeptides disclosed herein. Additional variants included as a feature of this disclosure are chimeric F2GF1 polypeptides that incorporate an F2 (e.g., comprising all or part of amino acids 24-107, numerically designated by alignment with SEQ ID NO:2) and/or F1 component (e.g., comprising all or part of amino acids 161-524, numerically designated by alignment with SEQ ID NO:2) from any of the exemplary sequences provided in FIG. 4 (either the same or different strain) and a G protein component (e.g., all or part of amino acids 149-229, numerically designated by alignment to SEQ ID NO:4) selected from any of the exemplary sequences provided in FIG. 5. Variants can arise through genetic drift, or can be produced artificially using site directed or random mutagenesis, or by recombination of two or more preexisting variants. For example, a variant F2GF1 polypeptide can include 1, or 2, or 5 or 10, or 15, or 50 or up to about 100 nucleotide differences as compared to the exemplary F2GF1 chimeras of SEQ ID NOs: 6, 8, 10, 12, 14, 16, 18 and 20.

A “domain” of a polypeptide or protein is a structurally defined element within the polypeptide or protein. In the context of this disclosure, a “furin cleavage domain” is a domain defined by cleavage of a precursor polypeptide by a furin protease. For example, the F protein is synthesized as a single polypeptide, designated F0. The F0 polypeptide is subsequently cleaved at two consensus furin recognition motifs by a furin protease to produce two structurally independent polypeptide units designated F2 and F1. F2 extends from amino acid 24 (following the signal peptide) to the first (in an N- to C-terminal direction) furin cleavage recognition site. F1 extends from the second furin cleavage site to the C-terminal end of the F0 polypeptide.

The terms “native” and “naturally occurring” refer to an element, such as a protein, polypeptide or nucleic acid, that is present in the same state as it is in nature. That is, the element has not been modified artificially. It will be understood, that in the context of this disclosure, there are numerous native/naturally occurring variants of RSV proteins or polypeptides, e.g., obtained from different naturally occurring strains or isolates of RSV.

The term “polypeptide” refers to a polymer in which the monomers are amino acid residues which are joined together through amide bonds. The terms “polypeptide” or “protein” as used herein are intended to encompass any amino acid sequence and include modified sequences such as glycoproteins. The term “polypeptide” is specifically intended to cover naturally occurring proteins, as well as those which are recombinantly or synthetically produced. The term “fragment,” in reference to a polypeptide, refers to a portion (that is, a subsequence) of a polypeptide. The term “immunogenic fragment” refers to all fragments of a polypeptide that retain at least one predominant immunogenic epitope of the full-length reference protein or polypeptide. Orientation within a polypeptide is generally recited in an N-terminal to C-terminal direction, defined by the orientation of the amino and carboxy moieties of individual amino acids. Polypeptides are translated from the N or amino-terminus towards the C or carboxy-terminus.

A “signal peptide” is a short amino acid sequence (e.g., approximately 18-25 amino acids in length) that direct newly synthesized secretory or membrane proteins to and through membranes, e.g., of the endoplasmic reticulum. Signal peptides are frequently but not universally located at the N-terminus of a polypeptide, and are frequently cleaved off by signal peptidases after the protein has crossed the membrane. Signal sequences typically contain three common structural features: an N-terminal polar basic region (n-region), a hydrophobic core, and a hydrophilic c-region).

The terms “polynucleotide” and “nucleic acid sequence” refer to a polymeric form of nucleotides at least 10 bases in length. Nucleotides can be ribonucleotides, deoxyribonucleotides, or modified forms of either nucleotide. The term includes single and double forms of DNA. By “isolated polynucleotide” is meant a polynucleotide that is not immediately contiguous with both of the coding sequences with which it is immediately contiguous (one on the 5′ end and one on the 3′ end) in the naturally occurring genome of the organism from which it is derived. In one embodiment, a polynucleotide encodes a polypeptide. The 5′ and 3′ direction of a nucleic acid is defined by reference to the connectivity of individual nucleotide units, and designated in accordance with the carbon positions of the deoxyribose (or ribose) sugar ring. The informational (coding) content of a polynucleotide sequence is read in a 5′ to 3′ direction.

A “recombinant” nucleic acid is one that has a sequence that is not naturally occurring or has a sequence that is made by an artificial combination of two otherwise separated segments of sequence. This artificial combination can be accomplished by chemical synthesis or, more commonly, by the artificial manipulation of isolated segments of nucleic acids, e.g., by genetic engineering techniques. A “recombinant” protein is one that is encoded by a heterologous (e.g., recombinant) nucleic acid, which has been introduced into a host cell, such as a bacterial or eukaryotic cell. The nucleic acid can be introduced, on an expression vector having signals capable of expressing the protein encoded by the introduced nucleic acid or the nucleic acid can be integrated into the host cell chromosome.

The term “purification” (e.g., with respect to a pathogen or a composition containing a pathogen) refers to the process of removing components from a composition, the presence of which is not desired. Purification is a relative term, and does not require that all traces of the undesirable component be removed from the composition. In the context of vaccine production, purification includes such processes as centrifugation, dialization, ion-exchange chromatography, and size-exclusion chromatography, affinity-purification or precipitation. Thus, the term “purified” does not require absolute purity; rather, it is intended as a relative term. Thus, for example, a purified nucleic acid preparation is one in which the specified protein is more enriched than the nucleic acid is in its generative environment, for instance within a cell or in a biochemical reaction chamber. A preparation of substantially pure nucleic acid or protein can be purified such that the desired nucleic acid represents at least 50% of the total nucleic acid content of the preparation. In certain embodiments, a substantially pure nucleic acid will represent at least 60%, at least 70%, at least 80%, at least 85%, at least 90%, or at least 95% or more of the total nucleic acid or protein content of the preparation.

An “isolated” biological component (such as a nucleic acid molecule, protein or organelle) has been substantially separated or purified away from other biological components in the cell of the organism in which the component naturally occurs, such as, other chromosomal and extra-chromosomal DNA and RNA, proteins and organelles. Nucleic acids and proteins that have been “isolated” include nucleic acids and proteins purified by standard purification methods. The term also embraces nucleic acids and proteins prepared by recombinant expression in a host cell as well as chemically synthesized nucleic acids and proteins.

An “antigen” is a compound, composition, or substance that can stimulate the production of antibodies and/or a T cell response in an animal, including compositions that are injected, absorbed or otherwise introduced into an animal. The term “antigen” includes all related antigenic epitopes. The term “epitope” or “antigenic determinant” refers to a site on an antigen to which B and/or T cells respond. The “predominant antigenic epitopes” are those epitopes to which a functionally significant host immune response, e.g., an antibody response or a T-cell response, is made. Thus, with respect to a protective immune response against a pathogen, the predominant antigenic epitopes are those antigenic moieties that when recognized by the host immune system result in protection from disease caused by the pathogen. The term “T-cell epitope” refers to an epitope that when bound to an appropriate MHC molecule is specifically bound by a T cell (via a T cell receptor). A “B-cell epitope” is an epitope that is specifically bound by an antibody (or B cell receptor molecule).

An “adjuvant” is an agent that enhances the production of an immune response in a non-specific manner. Common adjuvants include suspensions of minerals (alum, aluminum hydroxide, aluminum phosphate) onto which antigen is adsorbed; emulsions, including water-in-oil, and oil-in-water (and variants thereof, including double emulsions and reversible emulsions), liposaccharides, lipopolysaccharides, immunostimulatory nucleic acids (such as CpG oligonucleotides), liposomes, Toll Receptor agonists (particularly, TLR2, TLR4, TLR7/8 and TLR9 agonists), and various combinations of such components.

An “immunogenic composition” is a composition of matter suitable for administration to a human or animal subject that is capable of eliciting a specific immune response, e.g., against a pathogen, such as RSV. As such, an immunogenic composition includes one or more antigens (for example, polypeptide antigens) or antigenic epitopes. An immunogenic composition can also include one or more additional components capable of eliciting or enhancing an immune response, such as an excipient, carrier, and/or adjuvant. In certain instances, immunogenic compositions are administered to elicit an immune response that protects the subject against symptoms or conditions induced by a pathogen. In some cases, symptoms or disease caused by a pathogen is prevented (or reduced or ameliorated) by inhibiting replication of the pathogen (e.g., RSV) following exposure of the subject to the pathogen. In the context of this disclosure, the term immunogenic composition will be understood to encompass compositions that are intended for administration to a subject or population of subjects for the purpose of eliciting a protective or palliative immune response against RSV (that is, vaccine compositions or vaccines).

An “immune response” is a response of a cell of the immune system, such as a B cell, T cell, or monocyte, to a stimulus. An immune response can be a B cell response, which results in the production of specific antibodies, such as antigen specific neutralizing antibodies. An immune response can also be a T cell response, such as a CD4+ response or a CD8+ response. In some cases, the response is specific for a particular antigen (that is, an “antigen-specific response”). If the antigen is derived from a pathogen, the antigen-specific response is a “pathogen-specific response.” A “protective immune response” is an immune response that inhibits a detrimental function or activity of a pathogen, reduces infection by a pathogen, or decreases symptoms (including death) that result from infection by the pathogen. A protective immune response can be measured, for example, by the inhibition of viral replication or plaque formation in a plaque reduction assay or ELISA-neutralization assay, or by measuring resistance to pathogen challenge in vivo.

A “Th1” type immune response is characterized CD4+T helper cells that produce IL-2 and IFN-γ. In contrast, a “Th2” type immune response is characterized by CD4+ helper cells that produce IL-4, IL-5, and IL-13.

A “immunologically effective amount” is a quantity of a composition (typically, an immunogenic composition) used to elicit an immune response in a subject. Commonly, the desired result is the production of an antigen (e.g., pathogen)-specific immune response that is capable of or contributes to protecting the subject against the pathogen. However, to obtain a protective immune response against a pathogen can require multiple administrations of the immunogenic composition. Thus, in the context of this disclosure, the term immunologically effective amount encompasses a fractional dose that contributes in combination with previous or subsequent administrations to attaining a protective immune response.

The adjective “pharmaceutically acceptable” indicates that the subject is suitable for administration to a subject (e.g., a human or animal subject). Remington's Pharmaceutical Sciences, by E. W. Martin, Mack Publishing Co., Easton, Pa., 15th Edition (1975), describes compositions and formulations (including diluents) suitable for pharmaceutical delivery of therapeutic and/or prophylactic compositions, including immunogenic compositions.

“Solubility” is a measure the amount of a substance, in the context of this disclosure, a polypeptide, that will dissolve in a given amount of another substance, usually a liquid. Thus, an increase insolubility is an increase in the amount of a the polypeptide that remains without aggregating or separating from the substance (e.g., liquid) in which it is dissolved.

When referring to a polypeptide, “stability is a measure of the polypeptide's resistance to degradation. Thus, an increase in stability reflects an increase in the ability of the polypeptide to withstand degradation, for example, measured as an increased half-life in vivo, or an increased shelf life in vitro.

The term “modulate” in reference to a response, such as an immune response, means to alter or vary the onset, magnitude, duration or characteristics of the response. An agent that modulates an immune response alters at least one of the onset, magnitude, duration or characteristics of an immune response following its administration, or that alters at least one of the onset, magnitude, duration or characteristic as compared to a reference agent.

The term “reduces” is a relative term, such that an agent reduces a response or condition if the response or condition is quantitatively diminished following administration of the agent, or if it is diminished following administration of the agent, as compared to a reference agent. Similarly, the term “prevents” does not necessarily mean that an agent completely eliminates the response or condition, so long as at least one characteristic of the response or condition is eliminated. Thus, an immunogenic composition that reduces or prevents an infection or a response, such as a pathological response, e.g., vaccine enhanced viral disease, can, but does not necessarily completely eliminate such an infection or response, so long as the infection or response is measurably diminished, for example, by at least about 50%, such as by at least about 70%, or about 80%, or even by about 90% of (that is to 10% or less than) the infection or response in the absence of the agent, or in comparison to a reference agent.

A “subject” is a living multi-cellular vertebrate organism. In the context of this disclosure, the subject can be an experimental subject, such as a non-human animal, e.g., a mouse, a cotton rat, or a non-human primate. Alternatively, the subject can be a human subject.

### F2GF1 Chimeric RSV Antigens

The viral envelope of RSV includes virally encoded F, G and SH glycoproteins. The F and G glycoproteins are the only two components of the RSV virion that are known to induce RSV-specific neutralizing antibodies. The chimeric F2GF1 polypeptides disclosed herein were designed to incorporate structural features of the native F protein while simultaneously exhibiting important immunodominant epitopes of the RSV G protein. To facilitate folding and assembly during production, the two domains of the F protein produced by post-translational cleavage of the F0 precursor polypeptide by a furin protease (F1 and F2) were expressed in a single amino acid chain. The antigenic portion of the RSV G protein was incorporated between the F2 and F1 domains, taking into account the conformational distance constraints between F2 and F1. The design of these constructs was modeled based on the 3D model of the post-fusion state of the protein. This conformer has been predicted to be the most stable form of the protein.

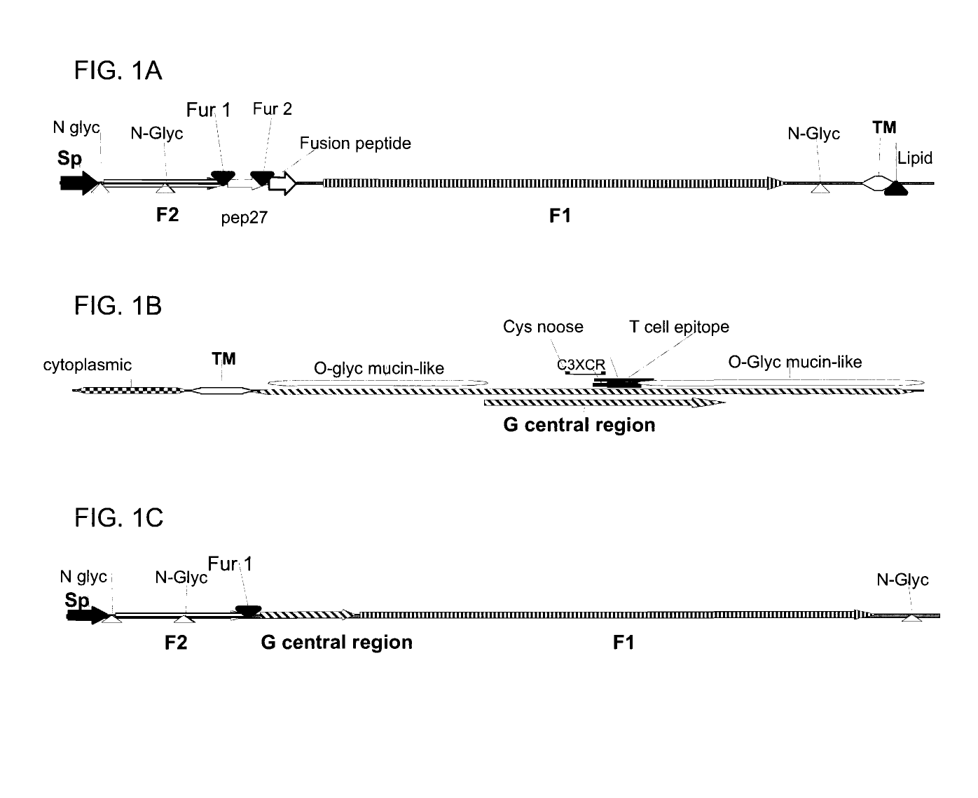

FIG. 1A schematically illustrates an exemplary RSV F protein and specific structural regions domains described herein. The F protein of RSV is translated as a single polypeptide precursor, designated F0. F0 folds and is subject to proteolysis and other post-translational modifications. First, a signal peptide (Sp) targets the translation of the nascent polypeptide to the reticulum endoplasmic (RE) and is later cleaved by a signal peptidase. The nascent polypeptide is then N-glycosylated in the RE at 3 sites represented by white triangles. F2 and F1 are generated by furin-cleavage (black inverted triangles) and folded together as a trimer of heterodimer (3 times F2-F1). Furin is a calcium-dependent serine endoprotease that can efficiently cleave precursor proteins at paired basic amino acid processing sites. Typically, such processing sites include a basic amino acid target sequence (canonically, Arg-X-(Arg/Lys)-Arg′). The RSV F protein includes two furin cleavage sites at positions 109 and 136. A description of furin processing of the RSV F protein, along with definitions of the art-accepted terminology is found in Zimmer et al. “Proteolytic activation of Respiratory Syncytial Virus fusion protein.” J. Biol. Chem. 276:31642-31650, 2001, and Zimmer et al., “Cleavage at the furin consensus sequence RAR/KR109 and presence of the intervening peptide of the Respiratory Syncytial Virus fusion protein are dispensable for virus replication in cell culture.” J. Virol. 76:9218-9224, 2002. The protein is anchored to the membrane using its transmembrane helix shown by the white lozenge (TM) in the C-terminal region. In addition, the RSV F protein features 15 Cysteines residues, 4 characterized neutralizing epitopes, 2 coiled-coil regions and a lipidation motif.

FIG. 1B schematically represents an exemplary RSV G protein (298 amino acids). The G protein is anchored to the virion membrane by its transmembrane hydrophobic region (amino acids 41-63). Amino acids 65-298 includes the portion of the G protein that is exposed at the surface of RSV. At each extremities are located highly O-glycosylated mucin-like regions. Five N-glycosylation motifs are also present in these two regions. The non-glycosylated central includes several important structural motifs, including: 1) a cysteine noose (aa173-190), which is the only portion of the G for which structural data are available; 2) an immunodominant MHC class II epitope at aa183-203; and 3) chemokine fractalkine receptor (C3XCR) and glycosaminoglycan (GAG) binding motifs, which are implicated in the process of viral attachment on the host cell surface.

This disclosure concerns chimeric RSV antigens that include in a N-terminal to C-terminal direction: a first polypeptide component corresponding to a subsequence of an RSV F protein; a polypeptide component including an immunodominant epitope of an RSV G protein; and a second polypeptide component corresponding to a subsequence of an RSV F protein. An exemplary F2GF1 polypeptide is schematically represented in FIG. 1C.

It will be evident to those of skill in the art that any RSV F and/or G protein sequences can be employed in the construction of recombinant chimeric RSV F2GF1 polypeptides. In the exemplary embodiments disclosed herein, the Long strain has been selected as a model. The sequence of the F protein, which is responsible for fusion of the virus envelope with the target cell membrane, is highly conserved among RSV isolates. In contrast, that of the G protein, which is responsible for virus attachment, is relatively variable. An alignment of RSV F and G protein sequences, illustrating identity and variation between the different proteins, are provided as FIGS. 4 and 5, respectively. Conserved and variable regions are readily apparent from these alignments.

In selecting F2 and F1 domains of the F protein, one of skill in the art will recognize that it is not strictly necessary to include the entire F2 and/or F1 domain. Typically, conformational considerations are of importance when selecting a subsequence (or fragment) of the F2 domain. Thus, the F2 domain typically includes a portion of the F2 domain that facilitates assembly and stability of the chimeric polypeptide. In certain exemplary variants, the F2 domain includes amino acids 24-107. Optionally, the F2 domain can include a signal peptide of the native F0 polypeptide (e.g., amino acids 1-23). Similarly, the F2 domain can optionally include additional amino acids, such as the pep27 domain. For example, in certain exemplary variants, the F2 domain includes amino acids 24-130.

Typically, at least a subsequence (or fragment) of the F1 domain is selected and designed to maintain a stable conformation that includes immunodominant epitopes of the F protein. For example, it is generally desirable to select a subsequence of the F1 polypeptide domain that includes epitopes recognized by neutralizing antibodies in the regions of amino acids 262-275 (palivizumab neutralization) and 423-436 (Centocor's ch101F MAb). Additionally, desirable to include T cell epitopes, e.g., in the region of amino acids 328-355. Most commonly, as a single contiguous portion of the F1 subunit (e.g., spanning amino acids 262-436) but epitopes could be retained in a synthetic sequence that includes these immunodominant epitopes as discontinuous elements assembled in a stable conformation. Thus, an F1 domain polypeptide comprises at least about amino acids 262-436 of an RSV F protein polypeptide. In one non-limiting example provided herein, the F1 domain comprises amino acids 161 to 524 of a native F protein polypeptide. In another non-limiting example, the F1 domain includes amino acids 151-524 of a native F protein polypeptide. One of skill in the art will recognize that additional shorter subsequences can be used at the discretion of the practitioner.

Similarly, the G protein polypeptide component is selected to include at least a subsequence (or fragment) of the G protein that retains the immunodominant T cell epitope(s), e.g., in the region of amino acids 183-197. Exemplary variants disclosed herein include, for example subsequences or fragments of the G protein that include amino acids 151-229, 149-229, or 128-229 of a native G protein. One of skill in the art will readily appreciate that longer or shorter portions of the G protein can also be used, so long as the portion selected does not conformationally destabilize or disrupt expression, folding or processing of the F2GF1 chimera. Optionally, the G protein domain includes an amino acid substitution at position 191, which has previously been shown to be involved in reducing and/or preventing enhanced disease characterized by eosinophilia associated with formalin inactivated RSV vaccines. A thorough description of the attributes of naturally occurring and substituted (N191A) G proteins can be found, e.g., in US Patent Publication No. 2005/0042230, which is incorporated herein by reference for all purposes.

If so desired, additional T cell epitopes can be identified using anchor motifs or other methods, such as neural net or polynomial determinations, known in the art, see, e.g., RANKPEP (available on the world wide web at: mifidfci.harvard.edu/Tools/rankpep.html); ProPredI (available on the world wide web at: imtech.res.in/raghava/propredI/index.html); Bimas (available on the world wide web at: www-bimas.dcrt.nih.gov/molbi/hla_bind/index.html); and SYFPEITH (available on the world wide web at: syfpeithi.bmi-heidelberg.com/scripts/MHCServer.dll/home.htm). For example, algorithms are used to determine the “binding threshold” of peptides, and to select those with scores that give them a high probability of MHC or antibody binding at a certain affinity. The algorithms are based either on the effects on MHC binding of a particular amino acid at a particular position, the effects on antibody binding of a particular amino acid at a particular position, or the effects on binding of a particular substitution in a motif-containing peptide. Within the context of an immunogenic peptide, a “conserved residue” is one which appears in a significantly higher frequency than would be expected by random distribution at a particular position in a peptide. Anchor residues are conserved residues that provide a contact point with the MHC molecule. T cell epitopes identified by such predictive methods can be confirmed by measuring their binding to a specific MHC protein and by their ability to stimulate T cells when presented in the context of the MHC protein.

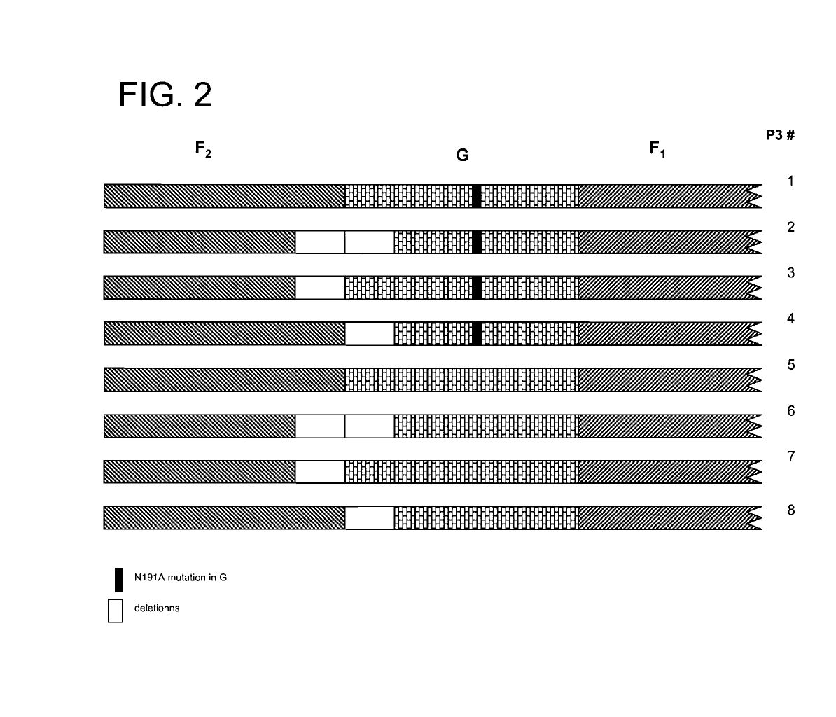

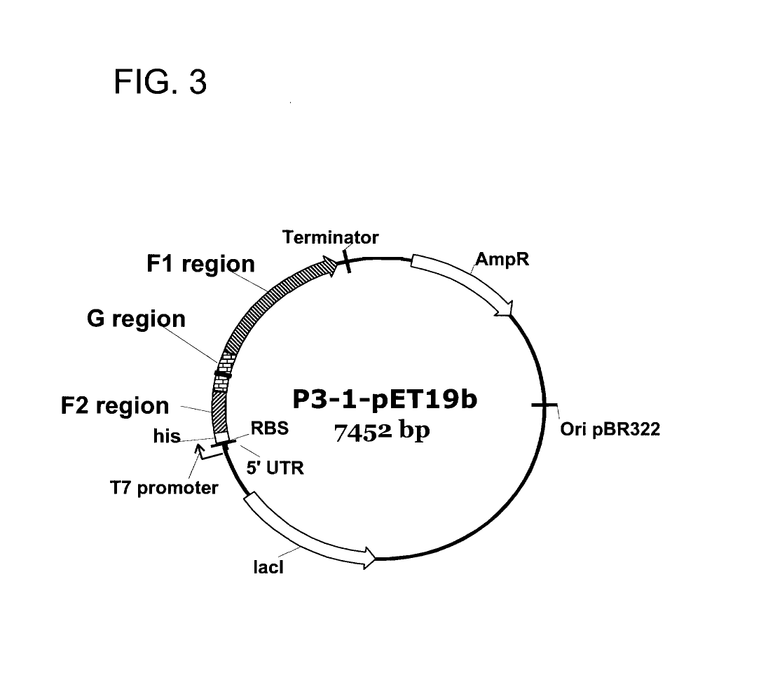

Eight exemplary prokaryotic variants were initially produced to demonstrate immunogenicity of chimeric F2GF1 polypeptide antigens. The following modifications were incorporated to enhance expression of the chimeric polypeptide. The native signal peptide, as well as the hydrophobic fusion peptide, and the C-terminal region of the protein starting from the transmembrane alpha helical structure, were removed. Exemplary F2GF1 chimeric RSV antigens are represented by SEQ ID NOs:6, 8, 10, 12, 14, 16, 18 and 20, which are schematically illustrated in FIG. 2. As shown in FIG. 2, these variants represent combinations of different subsequences of the F2 and G domains, such that subsequences extending from amino acid 24 through either amino acid 107 or 130 are combined with subsequences of the G protein extending from amino acid 149 to 229 or 128-229. P3-1, P3-2, P3-3 and P3-4 (SEQ ID NOs:6, 8, 10 and 12, respectively) include a single amino acid substitution at the position corresponding to amino acid position 191 of the native G protein, whereas, P3-5, P3-6, P3-7 and P3-8 include a naturally occurring asparagines at position 191. Additional details are provided below in the examples section.

Additional exemplary variants include chimeric F2GF1 polypeptides that are modified to remove specific cysteines that can be involved in the formation of disulfide bridges. There are 2 such cysteines in the F2 domain, 4 in the G domain, and 12 in the F1 domain. Accordingly variants can be produced that eliminate 1 or more of these cysteines, for example, by substituting the amino acid serine in place of one or more cysteines, e.g., at the positions corresponding to amino acids 40, 72, 291, 392, 401, 412, 422 and/or 518 of the P3-1 F2GF1 sequence. Alternatively, rather than substituting a serine (or another amino acid) for cysteine, hydrophobic residues (such as leucine, isoleucine, or valine) can be substituted for or near to cysteines. For example, the following amino acid substitutions replace one or more amino acids in the vicinity of positions 40 and 401 with one or more hydrophobic residues: Y36L, T39I, C40G, S41V and L400S, C4011.

Other exemplary embodiments are variants that have a deletion of one or more amino acids. For example, variants can be produced that omit a portion of the coiled coil structure at amino acids 51-66. Because the coiled coil structure is driven by hydrophobic interaction, reduction in the size of this structure is predicted to increase solubility of the chimeric polypeptide. Alternatively, variants can include additional amino acids. For example, the variants can include additional amino acids, that facilitate purification, (e.g., polyhistidine tags), or additional amino acids that increase stability, for example, stabilizing domains such as an isoleucine zipper domain.

In other examples, the polynucleotides that encode the F2GF1 chimeric RSV antigens are designed for and incorporated into expression vectors that are suitable for introduction and expression in eukaryotic (e.g., insect, plant, or mammalian cells). Favorably, such nucleic acids are codon optimized for expression in the selected vector/host cell. Exemplary eukaryotic chimeric F2GF1 polypeptides can be produced with minor differences as compared to the prokaryotic constructs described above. These modifications have been introduced to enhance expression and stability of the chimeric polypeptides when produced in a eukaryotic expression system, where glycosylation and other post-translational processing of the polypeptide can occur. For example, eukaryotic constructs are typically designed to include a signal peptide corresponding to the expression system, for example, a mammalian or viral signal peptide, such as the RSV F0 native signal sequence is favorably selected when expressing the chimeric polypeptide in mammalian cells. Alternatively, a signal peptide (such as a baculovirus signal peptide, or the melittin signal peptide, can be substituted for expression, in insect cells. Suitable plant signal peptides are known in the art, if a plant expression system is preferred. If desired, one or both furin cleavage sites can be removed to eliminate processing by furin protease in eukaryotic cells. Additionally, in the exemplary embodiments described herein, the G and F1 boundaries are slightly different from the boundaries of the prokaryotic constructs, showing additional suitable variations in F2GF1 polypeptide antigens. For example, in specific examples, the G peptide domain includes amino acids 152-229, instead of aa149-229 for the prokaryotic versions, and the F1 domain includes amino acids 151-524, instead of 161-524 present in the prokaryotic versions. Thus, this exemplary eukaryotic chimeric F2GF1 polypeptide includes the following sequence. From the N-terminus, the chimeric polypeptide includes amino acids 1-109 of the F0 polypeptide. There is a glycine linker at amino acid 110, followed by amino acids 152-229 of the G protein (either from a naturally occurring G protein, or incorporating a substitution of alanine in the place of asparagines at position 191) at positions 111-188. Following the G protein domain at positions 189-562 are amino acids 151-524 of the F1 domain. Thus, in this variant, the native pep27, fusion peptide and one or both furin recognition motifs are replaced by the G protein domain. It will be understood that any of the additional modifications can also be introduced into a eukaryotic F2GF1 chimeric polypeptide.

Nucleic Acids that Encode Chimeric F2GF1 Polypeptide Antigens

Another aspect of this disclosure concerns recombinant nucleic acids that encode the chimeric F2GF1 polypeptides described above. The recombinant nucleic acids include in a 5′ to 3′ direction, a first polynucleotide sequence that encodes at least a portion or fragment of an RSV F protein polypeptide furin cleavage domain 2 (F2 domain); a second polynucleotide sequence that encodes at least a portion or fragment of an RSV G protein polypeptide; and a third polynucleotide sequence that encodes at least a portion or fragment of an RSV F protein polypeptide furin cleavage domain 1 (F1 domain). The three component polynucleotide sequences are typically joined such that the encoded polypeptide segments are produced in a single contiguous chimeric polypeptide that includes in an N-terminal to C-terminal orientation: an F2 polypeptide component; a G protein component; and an F1 polypeptide component.

In certain embodiments, the recombinant nucleic acids are codon optimized for expression in a selected prokaryotic or eukaryotic host cell, such as a mammalian, plant or insect cell. To facilitate replication and expression, the nucleic acids can be incorporated into a vector, such as a prokaryotic or a eukaryotic expression vector. Although the nucleic acids disclosed herein can be included in any one of a variety of vectors (including, for example, bacterial plasmids; phage DNA; baculovirus; yeast plasmids; vectors derived from combinations of plasmids and phage DNA, viral DNA such as vaccinia, adenovirus, fowl pox virus, pseudorabies, adenovirus, adeno-associated virus, retroviruses and many others), most commonly the vector will be an expression vector suitable for generating polypeptide expression products. In an expression vector, the nucleic acid encoding the F2GF1 chimera is typically arranged in proximity and orientation to an appropriate transcription control sequence (promoter, and optionally, one or more enhancers) to direct mRNA synthesis. That is, the polynucleotide sequence of interest is operably linked to an appropriate transcription control sequence. Examples of such promoters include: the immediate early promoter of CMV, LTR or SV40 promoter, polyhedron promoter of baculovirus, E. coli lac or trp promoter, phage T7 and lambda P_{L} promoter, and other promoters known to control expression of genes in prokaryotic or eukaryotic cells or their viruses. The expression vector typically also contains a ribosome binding site for translation initiation, and a transcription terminator. The vector optionally includes appropriate sequences for amplifying expression. In addition, the expression vectors optionally comprise one or more selectable marker genes to provide a phenotypic trait for selection of transformed host cells, such as dihydrofolate reductase or neomycin resistance for eukaryotic cell culture, or such as tetracycline or ampicillin resistance in E. coli.

The expression vector can also include additional expression elements, for example, to improve the efficiency of translation. These signals can include, e.g., an ATG initiation codon and adjacent sequences. In some cases, for example, a translation initiation codon and associated sequence elements are inserted into the appropriate expression vector simultaneously with the polynucleotide sequence of interest (e.g., a native start codon). In such cases, additional translational control signals are not required. However, in cases where only a polypeptide coding sequence, or a portion thereof, is inserted, exogenous translational control signals, including an ATG initiation codon is provided for expression of the chimeric F2GF1 sequence. The initiation codon is placed in the correct reading frame to ensure translation of the polynucleotide sequence of interest. Exogenous transcriptional elements and initiation codons can be of various origins, both natural and synthetic. If desired, the efficiency of expression can be further increased by the inclusion of enhancers appropriate to the cell system in use (Scharf et al. (1994) Results Probl Cell Differ 20:125-62; Bitter et al. (1987) Methods in Enzymol 153:516-544).

Exemplary procedures sufficient to guide one of ordinary skill in the art through the production of recombinant F2GF1 nucleic acids can be found in Sambrook et al., Molecular Cloning: A Laboratory Manual, 2d ed., Cold Spring Harbor Laboratory Press, 1989; Sambrook et al., Molecular Cloning: A Laboratory Manual, 3d ed., Cold Spring Harbor Press, 2001; Ausubel et al., Current Protocols in Molecular Biology, Greene Publishing Associates, 1992 (and Supplements to 2003); and Ausubel et al., Short Protocols in Molecular Biology: A Compendium of Methods from Current Protocols in Molecular Biology, 4th ed., Wiley & Sons, 1999.

Exemplary nucleic acids that encode chimeric F2GF1 polypeptides are represented by SEQ ID NOs: 5, 7, 9, 11, 13, 15, 17, and 19. Additional variants of can be produced by assembling analogous F2, F1 and G protein polypeptide sequences selected from any of the known (or subsequently) discovered strains of RSV, e.g., as shown in FIGS. 4 and 5. Additional sequence variants that share sequence identity with the exemplary variants can be produced by those of skill in the art. Typically, the nucleic acid variants will encode polypeptides that differ by no more than 1%, or 2%, or 5%, or 10%, or 15%, or 20% of the nucleotide or amino acid residues. That is, the encoded polypeptides share at least 80%, or 85%, more commonly, at least about 90% or more, such as 95%, or even 98% or 99% sequence identity. It will be immediately understood by those of skill in the art, that the polynucleotide sequences encoding the F2GF1 polypeptides, can themselves share less sequence identity due to the redundancy of the genetic code.

It will be understood by those of skill in the art, that the similarity between chimeric F2GF1 polypeptide and polynucleotide sequences, as for polypeptide and nucleotide sequences in general, can be expressed in terms of the similarity between the sequences, otherwise referred to as sequence identity. Sequence identity is frequently measured in terms of percentage identity (or similarity); the higher the percentage, the more similar are the primary structures of the two sequences. In general, the more similar the primary structures of two amino acid (or polynucleotide) sequences, the more similar are the higher order structures resulting from folding and assembly. Variants of a chimeric F2GF1 polypeptide and polynucleotide sequences can have one or a small number of amino acid deletions, additions or substitutions but will nonetheless share a very high percentage of their amino acid, and generally their polynucleotide sequence.

Methods of determining sequence identity are well known in the art. Various programs and alignment algorithms are described in: Smith and Waterman, Adv. Appl. Math. 2:482, 1981; Needleman and Wunsch, J. Mol. Biol. 48:443, 1970; Higgins and Sharp, Gene 73:237, 1988; Higgins and Sharp, CABIOS 5:151, 1989; Corpet et al., Nucleic Acids Research 16:10881, 1988; and Pearson and Lipman, Proc. Natl. Acad. Sci. USA 85:2444, 1988. Altschul et al., Nature Genet. 6:119, 1994, presents a detailed consideration of sequence alignment methods and homology calculations. The NCBI Basic Local Alignment Search Tool (BLAST) (Altschul et al., J. Mol. Biol. 215:403, 1990) is available from several sources, including the National Center for Biotechnology Information (NCBI, Bethesda, Md.) and on the internet, for use in connection with the sequence analysis programs blastp, blastn, blastx, tblastn and tblastx. A description of how to determine sequence identity using this program is available on the NCBI website on the internet.

Another indicia of sequence similarity between two nucleic acids is the ability to hybridize. The more similar are the sequences of the two nucleic acids, the more stringent the conditions at which they will hybridize. The stringency of hybridization conditions are sequence-dependent and are different under different environmental parameters. Thus, hybridization conditions resulting in particular degrees of stringency will vary depending upon the nature of the hybridization method of choice and the composition and length of the hybridizing nucleic acid sequences. Generally, the temperature of hybridization and the ionic strength (especially the Na⁺ and/or Mg⁺⁺ concentration) of the hybridization buffer will determine the stringency of hybridization, though wash times also influence stringency. Generally, stringent conditions are selected to be about 5° C. to 20° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. Conditions for nucleic acid hybridization and calculation of stringencies can be found, for example, in Sambrook et al., Molecular Cloning: A Laboratory Manual, Cold Spring Harbor Laboratory Press, Cold Spring Harbor, N.Y., 2001; Tijssen, Hybridization With Nucleic Acid Probes, Part I: Theory and Nucleic Acid Preparation, Laboratory Techniques in Biochemistry and Molecular Biology, Elsevier Science Ltd., NY, N.Y., 1993 and Ausubel et al. Short Protocols in Molecular Biology, 4^{th} ed., John Wiley & Sons, Inc., 1999.

For purposes of the present disclosure, “stringent conditions” encompass conditions under which hybridization will only occur if there is less than 25% mismatch between the hybridization molecule and the target sequence. “Stringent conditions” can be broken down into particular levels of stringency for more precise definition. Thus, as used herein, “moderate stringency” conditions are those under which molecules with more than 25% sequence mismatch will not hybridize; conditions of “medium stringency” are those under which molecules with more than 15% mismatch will not hybridize, and conditions of “high stringency” are those under which sequences with more than 10% mismatch will not hybridize. Conditions of “very high stringency” are those under which sequences with more than 6% mismatch will not hybridize. In contrast nucleic acids that hybridize under “low stringency conditions include those with much less sequence identity, or with sequence identity over only short subsequences of the nucleic acid. It will, therefore, be understood that the various variants of nucleic acids that are encompassed by this disclosure are able to hybridize to at least on of SEQ ID NOs: 5, 7, 9, 11, 13, 15, 17, 19, 67 or 69, over substantially their entire length.

### Methods of Producing Chimeric RSV Antigenic Polypeptides

The F2GF1 chimeric RSV polypeptides disclosed herein are produced using well established procedures for the expression and purification of recombinant proteins. Procedures sufficient to guide one of skill in the art can be found in, for example, Sambrook and the Ausubel references cited above. Additional and specific details are provided hereinbelow.

Recombinant nucleic acids that encode the F2GF1 chimeric RSV antigens, such as (but not limited to) the exemplary nucleic acids represented by SEQ ID NOs:5, 7, 9, 11, 13, 15, 17, 19, 67 and/or 69, are introduced into host cells by any of a variety of well-known procedures, such as electroporation, liposome mediated transfection, Calcium phosphate precipitation, infection, transfection and the like, depending on the selection of vectors and host cells.

Host cells that include recombinant F2GF1 chimeric RSV antigen-encoding nucleic acids are, thus, also a feature of this disclosure. Favorable host cells include prokaryotic (i.e., bacterial) host cells, such as E. coli, as well as numerous eukaryotic host cells, including fungal (e.g., yeast, such as Saccharomyces cerevisiae and Picchia pastoris) cells, insect cells, plant cells, and mammalian cells (such as CHO cells). Recombinant F2GF1 nucleic acids are introduced (e.g., transduced, transformed or transfected) into host cells, for example, via a vector, such as an expression vector. As described above, the vector is most typically a plasmid, but such vectors can also be, for example, a viral particle, a phage, etc. Examples of appropriate expression hosts include: bacterial cells, such as E. coli, Streptomyces, and Salmonella typhimurium; fungal cells, such as Saccharomyces cerevisiae, Pichia pastoris, and Neurospora crassa; insect cells such as Drosophila and Spodoptera frugiperda; mammalian cells such as 3T3, COS, CHO, BHK, HEK 293 or Bowes melanoma; plant cells, including algae cells, etc.

The host cells can be cultured in conventional nutrient media modified as appropriate for activating promoters, selecting transformants, or amplifying the inserted polynucleotide sequences. The culture conditions, such as temperature, pH and the like, are typically those previously used with the host cell selected for expression, and will be apparent to those skilled in the art and in the references cited herein, including, e.g., Freshney (1994) Culture of Animal Cells, a Manual of Basic Technique, third edition, Wiley-Liss, New York and the references cited therein. Expression products corresponding to the nucleic acids of the invention can also be produced in non-animal cells such as plants, yeast, fungi, bacteria and the like. In addition to Sambrook, Berger and Ausubel, details regarding cell culture can be found in Payne et al. (1992) Plant Cell and Tissue Culture in Liquid Systems John Wiley & Sons, Inc. New York, N.Y.; Gamborg and Phillips (eds) (1995) Plant Cell, Tissue and Organ Culture; Fundamental Methods Springer Lab Manual, Springer-Verlag (Berlin Heidelberg N.Y.) and Atlas and Parks (eds) The Handbook of Microbiological Media (1993) CRC Press, Boca Raton, Fla.

In bacterial systems, a number of expression vectors can be selected depending upon the use intended for the expressed product. For example, when large quantities of a polypeptide or fragments thereof are needed for the production of antibodies, vectors which direct high level expression of fusion proteins that are readily purified are favorably employed. Such vectors include, but are not limited to, multifunctional E. coli cloning and expression vectors such as BLUESCRIPT (Stratagene), in which the coding sequence of interest, e.g., a polynucleotide of the invention as described above, can be ligated into the vector in-frame with sequences for the amino-terminal translation initiating Methionine and the subsequent 7 residues of beta-galactosidase producing a catalytically active beta galactosidase fusion protein; pIN vectors (Van Heeke & Schuster (1989) J Biol Chem 264:5503-5509); pET vectors (Novagen, Madison Wis.), in which the amino-terminal methionine is ligated in frame with a histidine tag; and the like.

Similarly, in yeast, such as Saccharomyces cerevisiae, a number of vectors containing constitutive or inducible promoters such as alpha factor, alcohol oxidase and PGH can be used for production of the desired expression products. For reviews, see Berger, Ausubel, and, e.g., Grant et al. (1987; Methods in Enzymology 153:516-544). In mammalian host cells, a number expression systems, including both plasmids and viral-based systems, can be utilized.

A host cell is optionally chosen for its ability to modulate the expression of the inserted sequences or to process the expressed protein in the desired fashion. Such modifications of the protein include, but are not limited to, glycosylation, (as well as, e.g., acetylation, carboxylation, phosphorylation, lipidation and acylation). Post-translational processing for example, which cleaves a precursor form into a mature form of the protein (for example, by a furin protease) is optionally performed in the context of the host cell. Different host cells such as 3T3, COS, CHO, HeLa, BHK, MDCK, 293, WI38, etc. have specific cellular machinery and characteristic mechanisms for such post-translational activities and can be chosen to ensure the correct modification and processing of the introduced, foreign protein.

For long-term, high-yield production of recombinant chimeric F2GF1 polypeptide encoded by the nucleic acids disclosed herein, stable expression systems are typically used. For example, cell lines which stably express a chimeric F2GF1 polypeptide are introduced into the host cell using expression vectors which contain viral origins of replication or endogenous expression elements and a selectable marker gene. Following the introduction of the vector, cells are allowed to grow for 1-2 days in an enriched media before they are switched to selective media. The purpose of the selectable marker is to confer resistance to selection, and its presence allows growth and recovery of cells which successfully express the introduced sequences. For example, resistant groups or colonies of stably transformed cells can be proliferated using tissue culture techniques appropriate to the cell type. Host cells transformed with a nucleic acid encoding a chimeric F2GF1 polypeptide are optionally cultured under conditions suitable for the expression and recovery of the encoded protein from cell culture.

Following transduction of a suitable host cell line and growth of the host cells to an appropriate cell density, the selected promoter is induced by appropriate means (e.g., temperature shift or chemical induction) and cells are cultured for an additional period. The secreted polypeptide product is then recovered from the culture medium. Alternatively, cells can be harvested by centrifugation, disrupted by physical or chemical means, and the resulting crude extract retained for further purification. Eukaryotic or microbial cells employed in expression of proteins can be disrupted by any convenient method, including freeze-thaw cycling, sonication, mechanical disruption, or use of cell lysing agents, or other methods, which are well know to those skilled in the art.

Expressed chimeric F2GF1 polypeptides can be recovered and purified from recombinant cell cultures by any of a number of methods well known in the art, including ammonium sulfate or ethanol precipitation, acid extraction, anion or cation exchange chromatography, phosphocellulose chromatography, hydrophobic interaction chromatography, affinity chromatography (e.g., using any of the tagging systems noted herein), hydroxylapatite chromatography, and lectin chromatography. Protein refolding steps can be used, as desired, in completing configuration of the mature protein. Finally, high performance liquid chromatography (HPLC) can be employed in the final purification steps. In addition to the references noted above, a variety of purification methods are well known in the art, including, e.g., those set forth in Sandana (1997) Bioseparation of Proteins, Academic Press, Inc.; and Bollag et al. (1996) Protein Methods, 2^{nd} Edition Wiley-Liss, NY; Walker (1996) The Protein Protocols Handbook Humana Press, NJ, Harris and Angal (1990) Protein Purification Applications: A Practical Approach IRL Press at Oxford, Oxford, U.K.; Scopes (1993) Protein Purification: Principles and Practice 3^{rd} Edition Springer Verlag, NY; Janson and Ryden (1998) Protein Purification Principles, High Resolution Methods and Applications, Second Edition Wiley-VCH, NY; and Walker (1998) Protein Protocols on CD-ROM Humana Press, NJ.

In certain examples, the nucleic acids are introduced into vectors suitable for introduction and expression in prokaryotic cells, e.g., E. coli cells. For example, a nucleic acid including a polynucleotide sequence that encodes a F2GF1 chimeric RSV antigen can be introduced into any of a variety of commercially available or proprietary vectors, such as the pET series of expression vectors (e.g., pET19b and pET21d). Expression of the coding sequence is inducible by IPTG, resulting in high levels of protein expression. The polynucleotide sequence encoding the chimeric RSV antigen is transcribed under the phage T7 promoter. Alternate vectors, such as pURV22 that include a heat-inducible lambda pL promoter are also suitable.

The expression vector is introduced (e.g., by electroporation) into a suitable bacterial host. Numerous suitable strains of E. coli are available and can be selected by one of skill in the art (for example, the Rosetta and BL21 (DE3) strains have proven favorable for expression of recombinant vectors containing polynucleotide sequences that encode F2GF1 chimeric RSV antigens.

In another example, the polynucleotides that encode the chimeric RSV antigens are cloned into a vector suitable for introduction into mammalian cells (e.g., CHO cells). In this exemplary embodiment, the polynucleotide sequence that encodes the chimeric RSV antigen is introduced into the pEE14 vector developed by Lonza Biologicals firm. The chimeric polypeptide is expressed under a constitutive promoter, the immediate early CMV (CytoMegaloVirus) promoter. Selection of the stably transfected cells expressing the chimer is made based on the ability of the transfected cells to grow in the absence of a glutamine source. Cells that have successfully integrated the pEE14 are able to grow in the absence of exogenous glutamine, because the pEE14 vector expresses the GS (Glutamine Synthetase) enzyme. Selected cells can be clonally expanded and characterized for expression of the chimeric polypeptide.

In another example, the polynucleotide sequence that encodes the F2GF1 chimeric RSV antigen is introduced into insect cells using a Baculovirus Expression Vector System (BEVS). Recombinant baculovirus capable of infecting insect cells can be generated using commercially available vectors, kits and/or systems, such as the BD BaculoGold system from BD BioScience. Briefly, the polynucleotide sequence encoding a F2GF1 chimeric RSV antigen is inserted into the pAcSG2 transfer vector. Then, host cells SF9 (Spodoptera frugiperda) are co-transfected by pAcSG2-chimer plasmid and BD BaculoGold, containing the linearized genomic DNA of the baculovirus Autographa californica nuclear polyhedrosis virus (AcNPV). Following transfection, homologous recombination occurs between the pACSG2 plasmid and the Baculovirus genome to generate the recombinant virus. In one example, the chimeric RSV antigen is expressed under the regulatory control of the polyhedrin promoter (pH). Similar transfer vectors can be produced using other promoters, such as the basic (Ba) and p10 promoters. Similarly, alternative insect cells can be employed, such as SF21 which is closely related to the Sf9, and the High Five (Hi5) cell line derived from a cabbage looper, Trichoplusia ni.

Following transfection and induction of expression (according to the selected promoter and/or enhancers or other regulatory elements), the expressed chimeric polypeptides are recovered (e.g., purified or enriched) and renatured to ensure folding into an antigenically active conformation. The following is an exemplary procedure for enrichment and renaturation of RSV F2GF1 chimeric antigens.

In an exemplary procedure for production from prokaryotic cells, RSV F2GF1 chimeric antigens are produced in bacterial (e.g., E. coli) cells. To facilitate purification, the F2GF1 chimeric antigens include a C-terminal or N-terminal his tag. In brief, the E. coli cell pellet is resuspended in lysis buffer and the cells are disrupted by sonication, French press, microfluidizer and/or emulsifier. The cell lysate is centrifuged between 10000 and 20000×g for 20 min at 4° C. and supernatant is discarded. The inclusion body (IB) pellet is resuspended in wash buffer and agitated at room temperature for at least 1 hour with 225 RPM agitation. The washed lysate is centrifuged between 10000 and 20000×g for 20 min at 4° C. and supernatant is discarded. Washed inclusion bodies are resuspended in solubilisation buffer (20 ml/g of IB) and incubated at room temperature for 4 hours with 225 RPM agitation. This mixture is then centrifuged at 20000×g for 20 min at 4° C. and pellet is discarded.

Solubilized inclusion bodies are loaded on an IMAC resin (Immobilized Metal Affinity Chromatography) previously equilibrated in IMAC loading buffer. The chimeric protein is then eluted from the column in IMAC eluting buffer. F2GF1 containing fractions are pooled, and the pooled fractions are concentrated on an ultrafiltration membrane for a size exclusion chromatography step. The concentrated IMAC pool is loaded on a size exclusion chromatography column equilibrated with SEC buffer, and the chimeric protein is eluted in the same buffer. Eluted fractions containing F2GF1 protein are again pooled, then quantified by absorbance at 280 nm, aliquoted and frozen at −20° C. until renaturation.

The following is an exemplary procedure for the renaturation of RSV F2GF1 chimeric antigens. F2GF1 protein concentration is brought to 1 mg/ml by dilution in SEC buffer. The protein is diafiltered in pre-refolding buffer to decrease lauroylsarcosine concentration up to 0.1% using tangential flow filtration (TFF). Protein at 1 mg/ml in pre-refolding buffer is rapidly diluted 10 times in pre-chilled refolding buffer, and the resulting mixture is stirred for 30 minutes at 4° C., then incubated without stirring overnight at 4° C.

During the subsequent renaturation process the chimeric protein is maintained at 4° C. until use or freezing. After the overnight incubation, the mixture is concentrated 10× by TFF. Resulting retentate volume is diafiltered with the same TFF cartridge with 5-10 volumes of 1M arginine refolding buffer, keeping the volume constant. The resulting retentate is then diafiltered with 5-10 volumes of final 300 mM arginine refolding buffer, again maintaining a constant volume. The retentate is then centrifuged at 20000×g for 20 min at 4° C., and the supernatant is harvested. Protein concentration is determined using the RCDC assay from BioRad (modified Lowry colorimetric assay). Renatured F2GF1 is aliquoted and stored at −20° C. for in vitro and/or in vivo use.

Table 1 provides a description of the buffers used during the purification and renaturation process.

Alternative excipients for renaturation, which are also suitable for inclusion in immunogenic compositions for administration to animal (e.g., human) subjects are further described below.

TABLE 1
Buffer compositions.

| Lysis buffer | Wash buffer | Solubilisation buffer |
|---|---|---|
| 50 mM Tris | 50 mM Tris | 50 mM Tris |
| 20 mM TCEP | 10-20 mM TCEP | 5%-30% lauroylsarcosine |
| 20 mM EDTA | 5 mM EDTA | 5% glycerol |
| pH 8.0 | 2% Triton X-100 | 5-20 mM TCEP |
| | pH 8.0 | 0.5 mM EDTA |
| | | pH 8.0 |
| IMAC loading buffer | IMAC eluting buffer | SEC buffer |
| 50 mM Tris | 50 mM Tris | 50 mM Tris |
| 2% lauroylsarcosine | 2% lauroylsarcosine | 2% lauroylsarcosine |
| 5% glycerol | 5% glycerol | 5% glycerol |
| 5-20 mM TCEP | 5-20 mM TCEP | 5-20 mM TCEP |
| pH 8.5 | 500 mM imidazole | pH 8.5 |
| | pH 8.5 | |

| Pre-refolding buffer | Refolding buffer |
|---|---|
| 10 mM Tris | 50 mM Tris |
| 0.05 mM EDTA | 250-500 mM NaCl |
| 1 mM TCEP | 270-1000 mM sucrose |
| 0.06%-0.1% lauroylsarcosine | 1 mM EDTA |
| pH 8.5 | 500-1000 mM L-arginine |
| | 3.8-10 mM reduced glutathione (GSH) |
| | 1.2-10 mM oxidized glutathione (GSSG) |
| | pH 8.5 |
| 1M arginine refolding buffer | 300 mM arginine refolding buffer |
| 50 mM Tris | 50 mM Tris |
| 250-500 mM NaCl | 250 mM NaCl |
| 270-1000 mM sucrose | 270-1000 mM sucrose |
| 1 mM EDTA | 1 mM EDTA |
| 1M L-arginine | 100-300 mM L-arginine |
| 3.8-10 mM reduced | 3.8-10 mM reduced glutathione (GSH) |
| glutathione (GSH) | |
| 1.2-10 mM oxidized | 1.2-10 mM oxidized glutathione (GSSG) |
| glutathione (GSSG) | |
| pH 8.5 | pH 8.5 |

### Immunogenic Compositions and Methods

Also provided are immunogenic compositions including a chimeric RSV F2GF1 antigen and a pharmaceutically acceptable diluent, carrier or excipient. Numerous pharmaceutically acceptable diluents and carriers and/or pharmaceutically acceptable excipients are known in the art and are described, e.g., in Remington's Pharmaceutical Sciences, by E. W. Martin, Mack Publishing Co., Easton, Pa., 15th Edition (1975).

In general, the nature of the diluent, carrier and/or excipient will depend on the particular mode of administration being employed. For instance, parenteral formulations usually include injectable fluids that include pharmaceutically and physiologically acceptable fluids such as water, physiological saline, balanced salt solutions, aqueous dextrose, glycerol or the like as a vehicle. In certain formulations (for example, solid compositions, such as powder forms), a liquid diluent is not employed. In such formulations, non-toxic solid carriers can be used, including for example, pharmaceutical grades of trehalose, mannitol, lactose, starch or magnesium stearate.

Accordingly, suitable excipients and carriers can be selected by those of skill in the art to produce a formulation suitable for delivery to a subject by a selected route of administration.

Particular examples are given above in Table 1. Additional excipients include, without limitation: glycerol, polyethylene glycol (PEG), glass forming polyols (such as, sorbitol, trehalose) N-lauroylsarcosine (e.g., sodium salt), L proline, non detergent sulfobetaine, guanidine hydrochloride, urea, trimethylamine oxide, KCl, Ca²⁺, Mg²⁺, Mn²⁺, Zn²⁺, (and other divalent cation related salts), dithiothreitol (DTT), dithioerytrol, β-mercaptoethanol, Detergents (including, e.g., Tween80, Tween20, Triton X-100, NP-40, Empigen BB, Octylglucoside, Lauroyl maltoside, Zwittergent 3-08, Zwittergent 3-10, Zwittergent 3-12, Zwittergent 3-14, Zwittergent 3-16, CHAPS, sodium deoxycholate, sodium dodecyl sulphate, and cetyltrimethylammonium bromide.

In certain favorable examples, the immunogenic composition also includes an adjuvant. Suitable adjuvants for use in immunogenic compositions containing chimeric F2GF1 polypeptides are adjuvants that in combination with the F2GF1 antigens disclosed herein are safe and minimally reactogenic when administered to a subject.

One suitable adjuvant for use in combination with F2GF1 chimeric antigens is a non-toxic bacterial lipopolysaccharide derivative. An example of a suitable non-toxic derivative of lipid A, is monophosphoryl lipid A or more particularly 3-Deacylated monophoshoryl lipid A (3D-MPL). 3D-MPL is sold under the name MPL by GlaxoSmithKline Biologicals N.A., and is referred throughout the document as MPL or 3D-MPL. See, for example, U.S. Pat. Nos. 4,436,727; 4,877,611; 4,866,034 and 4,912,094. 3D-MPL primarily promotes CD4+T cell responses with an IFN-γ (Th1) phenotype. 3D-MPL can be produced according to the methods disclosed in GB2220211 A. Chemically it is a mixture of 3-deacylated monophosphoryl lipid A with 3, 4, 5 or 6 acylated chains. In the compositions of the present invention small particle 3D-MPL can be used. Small particle 3D-MPL has a particle size such that it can be sterile-filtered through a 0.22 μm filter. Such preparations are described in WO94/21292.

Said lipopolysaccharide, such as 3D-MPL, can be used at amounts between 1 and 50 μg, per human dose of the immunogenic composition. Such 3D-MPL can be used at a level of about 25 μg, for example between 20-30 μg, suitably between 2′-29 μg or between 22 and 28 μg or between 23 and 27 μg or between 24 and 26 μg, or 25 μg. In another embodiment, the human dose of the immunogenic composition comprises 3D-MPL at a level of about 10 μg, for example between 5 and 15 μg, suitably between 6 and 14 μg, for example between 7 and 13 μg or between 8 and 12 μg or between 9 and 11 μg, or 10 μg. In a further embodiment, the human dose of the immunogenic composition comprises 3D-MPL at a level of about 5 μg, for example between 1 and 9 μg, or between 2 and 8 μg or suitably between 3 and 7 μg or 4 and μg, or 5 μg.

In other embodiments, the lipopolysaccharide can be a β(1-6) glucosamine disaccharide, as described in U.S. Pat. No. 6,005,099 and EP Patent No. 0 729 473 B1. One of skill in the art would be readily able to produce various lipopolysaccharides, such as 3D-MPL, based on the teachings of these references. Nonetheless, each of these references is incorporated herein by reference. In addition to the aforementioned immunostimulants (that are similar in structure to that of LPS or MPL or 3D-MPL), acylated monosaccharide and disaccharide derivatives that are a sub-portion to the above structure of MPL are also suitable adjuvants. In other embodiments, the adjuvant is a synthetic derivative of lipid A, some of which are described as TLR-4 agonists, and include, but are not limited to:

OM174 (2-deoxy-6-o-[2-deoxy-2-[(R)-3-dodecanoyloxytetra-decanoylamino]-4-o-phosphono-β-D-glucopyranosyl]-2-[(R)-3-hydroxytetradecanoylamino]-α-D-glucopyranosyldihydrogenphosphate), (WO 95/14026)

OM 294 DP (3S,9R)-3-[(R)-dodecanoyloxytetradecanoylamino]-4-oxo-5-aza-9(R)-[(R)-3-hydroxytetradecanoylamino]decan-1,10-diol,1,10-bis(dihydrogenophosphate) (WO 99/64301 and WO 00/0462)

OM 197 MP-Ac DP (3S—, 9R)-3-[(R)-dodecanoyloxytetradecanoylamino]-4-oxo-5-aza-9-[(R)-3-hydroxytetradecanoylamino]decan-1,10-diol,1-dihydrogenophosphate 10-(6-aminohexanoate) (WO 01/46127)

Other TLR4 ligands which can be used are alkyl Glucosaminide phosphates (AGPs) such as those disclosed in WO 98/50399 or U.S. Pat. No. 6,303,347 (processes for preparation of AGPs are also disclosed), suitably RC527 or RC529 or pharmaceutically acceptable salts of AGPs as disclosed in U.S. Pat. No. 6,764,840. Some AGPs are TLR4 agonists, and some are TLR4 antagonists. Both are thought to be useful as adjuvants.

Other suitable TLR-4 ligands, capable of causing a signaling response through TLR-4 (Sabroe et al, JI 2003 p1630-5) are, for example, lipopolysaccharide from gram-negative bacteria and its derivatives, or fragments thereof, in particular a non-toxic derivative of LPS (such as 3D-MPL). Other suitable TLR agonists are: heat shock protein (HSP) 10, 60, 65, 70, 75 or 90; surfactant Protein A, hyaluronan oligosaccharides, heparan sulphate fragments, fibronectin fragments, fibrinogen peptides and b-defensin-2, and muramyl dipeptide (MDP). In one embodiment the TLR agonist is HSP 60, 70 or 90. Other suitable TLR-4 ligands are as described in WO 2003/011223 and in WO 2003/099195, such as compound I, compound II and compound III disclosed on pages 4-5 of WO2003/011223 or on pages 3-4 of WO2003/099195 and in particular those compounds disclosed in WO2003/011223 as ER803022, ER803058, ER803732, ER804053, ER804057, ER804058, ER804059, ER804442, ER804680, and ER804764. For example, one suitable TLR-4 ligand is ER804057.

Additional TLR agonists are also useful as adjuvants. The term “TLR agonist” refers to an agent that is capable of causing a signaling response through a TLR signaling pathway, either as a direct ligand or indirectly through generation of endogenous or exogenous ligand. Such natural or synthetic TLR agonists can be used as alternative or additional adjuvants. A brief review of the role of TLRs as adjuvant receptors is provided in Kaisho & Akira, Biochimica et Biophysica Acta 1589:1-13, 2002. These potential adjuvants include, but are not limited to agonists for TLR2, TLR3, TLR7, TLR8 and TLR9. Accordingly, in one embodiment, the adjuvant and immunogenic composition further comprises an adjuvant which is selected from the group consisting of: a TLR-1 agonist, a TLR-2 agonist, TLR-3 agonist, a TLR-4 agonist, TLR-5 agonist, a TLR-6 agonist, TLR-7 agonist, a TLR-8 agonist, TLR-9 agonist, or a combination thereof.

In one embodiment of the present invention, a TLR agonist is used that is capable of causing a signaling response through TLR-1. Suitably, the TLR agonist capable of causing a signaling response through TLR-1 is selected from: Tri-acylated lipopeptides (LPs); phenol-soluble modulin; Mycobacterium tuberculosis LP; S-(2,3-bis(palmitoyloxy)-(2—RS)-propyl)-N-palmitoyl-(R)-Cys-(S)-Ser-(S)-Lys(4)-OH, trihydrochloride (Pam3Cys) LP which mimics the acetylated amino terminus of a bacterial lipoprotein and OspA LP from Borrelia burgdorfei.

In an alternative embodiment, a TLR agonist is used that is capable of causing a signaling response through TLR-2. Suitably, the TLR agonist capable of causing a signaling response through TLR-2 is one or more of a lipoprotein, a peptidoglycan, a bacterial lipopeptide from M tuberculosis, B burgdorferi or T pallidum; peptidoglycans from species including Staphylococcus aureus; lipoteichoic acids, mannuronic acids, Neisseria porins, bacterial fimbriae, Yersina virulence factors, CMV virions, measles haemagglutinin, and zymosan from yeast.

In an alternative embodiment, a TLR agonist is used that is capable of causing a signaling response through TLR-3. Suitably, the TLR agonist capable of causing a signaling response through TLR-3 is double stranded RNA (dsRNA), or polyinosinic-polycytidylic acid (Poly IC), a molecular nucleic acid pattern associated with viral infection.

In an alternative embodiment, a TLR agonist is used that is capable of causing a signaling response through TLR-5. Suitably, the TLR agonist capable of causing a signaling response through TLR-5 is bacterial flagellin.

In an alternative embodiment, a TLR agonist is used that is capable of causing a signaling response through TLR-6. Suitably, the TLR agonist capable of causing a signaling response through TLR-6 is mycobacterial lipoprotein, di-acylated LP, and phenol-soluble modulin. Additional TLR6 agonists are described in WO 2003/043572.

In an alternative embodiment, a TLR agonist is used that is capable of causing a signaling response through TLR-7. Suitably, the TLR agonist capable of causing a signaling response through TLR-7 is a single stranded RNA (ssRNA), loxoribine, a guanosine analogue at positions N7 and C8, or an imidazoquinoline compound, or derivative thereof. In one embodiment, the TLR agonist is imiquimod. Further TLR7 agonists are described in WO 2002/085905.

In an alternative embodiment, a TLR agonist is used that is capable of causing a signaling response through TLR-8. Suitably, the TLR agonist capable of causing a signaling response through TLR-8 is a single stranded RNA (ssRNA), an imidazoquinoline molecule with anti-viral activity, for example resiquimod (R848); resiquimod is also capable of recognition by TLR-7. Other TLR-8 agonists which can be used include those described in WO 2004/071459.

In an alternative embodiment, a TLR agonist is used that is capable of causing a signaling response through TLR-9. In one embodiment, the TLR agonist capable of causing a signaling response through TLR-9 is HSP90. Alternatively, the TLR agonist capable of causing a signaling response through TLR-9 is bacterial or viral DNA, DNA containing unmethylated CpG nucleotides, in particular sequence contexts known as CpG motifs. CpG-containing oligonucleotides induce a predominantly Th1 response. Such oligonucleotides are well known and are described, for example, in WO 96/02555, WO 99/33488 and U.S. Pat. Nos. 6,008,200 and 5,856,462. Suitably, CpG nucleotides are CpG oligonucleotides. Suitable oligonucleotides for use in the immunogenic compositions of the present invention are CpG containing oligonucleotides, optionally containing two or more dinucleotide CpG motifs separated by at least three, suitably at least six or more nucleotides. A CpG motif is a Cytosine nucleotide followed by a Guanine nucleotide. The CpG oligonucleotides of the present invention are typically deoxynucleotides. In a specific embodiment the internucleotide in the oligonucleotide is phosphorodithioate, or suitably a phosphorothioate bond, although phosphodiester and other internucleotide bonds are within the scope of the invention. Also included within the scope of the invention are oligonucleotides with mixed internucleotide linkages. Methods for producing phosphorothioate oligonucleotides or phosphorodithioate are described in U.S. Pat. Nos. 5,666,153, 5,278,302 and WO 95/26204.

Other adjuvants that can be used in immunogenic compositions with a chimeric F2GF1 polypeptide, e.g., on their own or in combination with 3D-MPL, or another adjuvant described herein, are saponins, such as QS21.

Saponins are taught in: Lacaille-Dubois, M and Wagner H. (1996. A review of the biological and pharmacological activities of saponins. Phytomedicine vol 2 pp 363-386). Saponins are steroid or triterpene glycosides widely distributed in the plant and marine animal kingdoms. Saponins are noted for forming colloidal solutions in water which foam on shaking, and for precipitating cholesterol. When saponins are near cell membranes they create pore-like structures in the membrane which cause the membrane to burst. Haemolysis of erythrocytes is an example of this phenomenon, which is a property of certain, but not all, saponins.

Saponins are known as adjuvants in vaccines for systemic administration. The adjuvant and haemolytic activity of individual saponins has been extensively studied in the art (Lacaille-Dubois and Wagner, supra). For example, Quil A (derived from the bark of the South American tree Quillaja Saponaria Molina), and fractions thereof, are described in U.S. Pat. No. 5,057,540 and “Saponins as vaccine adjuvants”, Kensil, C. R., Crit. Rev Ther Drug Carrier Syst, 1996, 12 (1-2):1-55; and EP 0 362 279 B1. Particulate structures, termed Immune Stimulating Complexes (ISCOMS), comprising fractions of Quil A are haemolytic and have been used in the manufacture of vaccines (Morein, B., EP 0 109 942 B1; WO 96/11711; WO 96/33739). The haemolytic saponins QS21 and QS17 (HPLC purified fractions of Quil A) have been described as potent systemic adjuvants, and the method of their production is disclosed in U.S. Pat. No. 5,057,540 and EP 0 362 279 B1, which are incorporated herein by reference. Other saponins which have been used in systemic vaccination studies include those derived from other plant species such as Gypsophila and Saponaria (Bomford et al., Vaccine, 10(9):572-577, 1992). QS21 is an Hplc purified non-toxic fraction derived from the bark of Quillaja Saponaria Molina. A method for producing QS21 is disclosed in U.S. Pat. No. 5,057,540. Non-reactogenic adjuvant formulations containing QS21 are described in WO 96/33739. The aforementioned references are incorporated by reference herein. Said immunologically active saponin, such as QS21, can be used in amounts of between 1 and 50 μg, per human dose of the immunogenic composition. Advantageously QS21 is used at a level of about 25 μg, for example between 20-30 μg, suitably between 21-29 μg or between 22-28 μg or between 23-27 μg or between 24-26 μg, or 25 μg. In another embodiment, the human dose of the immunogenic composition comprises QS21 at a level of about 10 μg, for example between 5 and 15 μg, suitably between 6-14 μg, for example between 7-13 μg or between 8-12 μg or between 9-11 μg, or 10 μg. In a further embodiment, the human dose of the immunogenic composition comprises QS21 at a level of about 5 μg, for example between 1-9 μg, or between 2-8 μg or suitably between 3-7 μg or 4-6 μg, or 5 μg. Such formulations comprising QS21 and cholesterol have been shown to be successful Th1 stimulating adjuvants when formulated together with an antigen. Thus, for example, chimeric F2GF1 polypeptides can favorably be employed in immunogenic compositions with an adjuvant comprising a combination of QS21 and cholesterol.

Optionally, the adjuvant can also include mineral salts such as an aluminium or calcium salts, in particular aluminium hydroxide, aluminium phosphate and calcium phosphate. For example, an adjuvant containing 3D-MPL in combination with an aluminium salt (e.g., aluminium hydroxide or “alum”) is suitable for formulation in an immunogenic composition containing a chimeric F2GF1 polypeptide for administration to a human subject.

Another class of suitable Th1 biasing adjuvants for use in formulations with chimeric F2GF1 polypeptides include OMP-based immunostimulatory compositions. OMP-based immunostimulatory compositions are particularly suitable as mucosal adjuvants, e.g., for intranasal administration. OMP-based immunostimulatory compositions are a genus of preparations of outer membrane proteins (OMPs, including some porins) from Gram-negative bacteria, such as, but not limited to, Neisseria species (see, e.g., Lowell et al., J. Exp. Med. 167:658, 1988; Lowell et al., Science 240:800, 1988; Lynch et al., Biophys. J. 45:104, 1984; Lowell, in “New Generation Vaccines” 2nd ed., Marcel Dekker, Inc., New York, Basil, Hong Kong, page 193, 1997; U.S. Pat. No. 5,726,292; U.S. Pat. No. 4,707,543), which are useful as a carrier or in compositions for immunogens, such as bacterial or viral antigens. Some OMP-based immunostimulatory compositions can be referred to as “Proteosomes,” which are hydrophobic and safe for human use. Proteosomes have the capability to auto-assemble into vesicle or vesicle-like OMP clusters of about 20 nm to about 800 nm, and to noncovalently incorporate, coordinate, associate (e.g., electrostatically or hydrophobically), or otherwise cooperate with protein antigens (Ags), particularly antigens that have a hydrophobic moiety. Any preparation method that results in the outer membrane protein component in vesicular or vesicle-like form, including multi-molecular membranous structures or molten globular-like OMP compositions of one or more OMPs, is included within the definition of Proteosome. Proteosomes can be prepared, for example, as described in the art (see, e.g., U.S. Pat. No. 5,726,292 or U.S. Pat. No. 5,985,284). Proteosomes cam also contain an endogenous lipopolysaccharide or lipooligosaccharide (LPS or LOS, respectively) originating from the bacteria used to produce the OMP porins (e.g., Neisseria species), which generally will be less than 2% of the total OMP preparation.

Proteosomes are composed primarily of chemically extracted outer membrane proteins (OMPs) from Neisseria menigitidis (mostly porins A and B as well as class 40MP), maintained in solution by detergent (Lowell G H. Proteosomes for Improved Nasal, Oral, or Injectable Vaccines. In: Levine M M, Woodrow G C, Kaper J B, Cobon G S, eds, New Generation Vaccines. New York: Marcel Dekker, Inc. 1997; 193-206). Proteosomes can be formulated with a variety of antigens such as purified or recombinant proteins derived from viral sources, including the chimeric F2GF1 polypeptides disclosed herein, e.g., by diafiltration or traditional dialysis processes. The gradual removal of detergent allows the formation of particulate hydrophobic complexes of approximately 100-200 nm in diameter (Lowell GH. Proteosomes for Improved Nasal, Oral, or Injectable Vaccines. In: Levine M M, Woodrow G C, Kaper J B, Cobon G S, eds, New Generation Vaccines. New York: Marcel Dekker, Inc. 1997; 193-206).

“Proteosome: LPS or Protollin” as used herein refers to preparations of proteosomes admixed, e.g., by the exogenous addition, with at least one kind of lipo-polysaccharide to provide an OMP-LPS composition (which can function as an immunostimulatory composition). Thus, the OMP-LPS composition can be comprised of two of the basic components of Protollin, which include (1) an outer membrane protein preparation of Proteosomes (e.g., Projuvant) prepared from Gram-negative bacteria, such as Neisseria meningitidis, and (2) a preparation of one or more liposaccharides. A lipo-oligosaccharide can be endogenous (e.g., naturally contained with the OMP Proteosome preparation), can be admixed or combined with an OMP preparation from an exogenously prepared lipo-oligosaccharide (e.g., prepared from a different culture or microorganism than the OMP preparation), or can be a combination thereof. Such exogenously added LPS can be from the same Gram-negative bacterium from which the OMP preparation was made or from a different Gram-negative bacterium. Protollin should also be understood to optionally include lipids, glycolipids, glycoproteins, small molecules, or the like, and combinations thereof. The Protollin can be prepared, for example, as described in U.S. Patent Application Publication No. 2003/0044425.

Combinations of different adjuvants, such as those mentioned hereinabove, can also be used in compositions with chimeric F2GF1 polypeptides. For example, as already noted, QS21 can be formulated together with 3D-MPL. The ratio of QS21:3D-MPL will typically be in the order of 1:10 to 10:1; such as 1:5 to 5:1, and often substantially 1:1. Typically, the ratio is in the range of 2.5:1 to 1:1 3D-MPL:QS21. Another combination adjuvant formulation includes 3D-MPL and an aluminium salt, such as aluminium hydroxide. When formulated in combination, this combination can enhance an antigen-specific Th1 immune response.

In some instances, the adjuvant formulation includes an oil-in-water emulsion, or a mineral salt such as a calcium or aluminium salt, for example calcium phosphate, aluminium phosphate or aluminium hydroxide.

One example of an oil-in-water emulsion comprises a metabolisable oil, such as squalene, a tocol such as alpha-tocopherol, and a surfactant, such as polysorbate 80 or Tween 80, in an aqueous carrier, and does not contain any additional immunostimulants(s), in particular it does not contain a non-toxic lipid A derivative (such as 3D-MPL) or a saponin (such as QS21). The aqueous carrier can be, for example, phosphate buffered saline. Additionally the oil-in-water emulsion can contain span 85 and/or lecithin and/or tricaprylin.

In another embodiment of the invention there is provided a vaccine composition comprising an antigen or antigen composition and an adjuvant composition comprising an oil-in-water emulsion and optionally one or more further immunostimulants, wherein said oil-in-water emulsion comprises 0.5-10 mg metabolisable oil (suitably squalene), 0.5-11 mg tocol (suitably alpha-tocopherol) and 0.4-4 mg emulsifying agent.

In one specific embodiment, the adjuvant formulation includes 3D-MPL prepared in the form of an emulsion, such as an oil-in-water emulsion. In some cases, the emulsion has a small particle size of less than 0.2 μm in diameter, as disclosed in WO 94/21292. For example, the particles of 3D-MPL can be small enough to be sterile filtered through a 0.22 micron membrane (as described in European Patent number 0 689 454). Alternatively, the 3D-MPL can be prepared in a liposomal formulation. Optionally, the adjuvant containing 3D-MPL (or a derivative thereof) also includes an additional immunostimulatory component.

For example, when an immunogenic composition with a chimeric F2GF1 polypeptide antigen is formulated for administration to an infant, the dosage of adjuvant is determined to be effective and relatively non-reactogenic in an infant subject. Generally, the dosage of adjuvant in an infant formulation is lower than that used in formulations designed for administration to adult (e.g., adults aged 65 or older). For example, the amount of 3D-MPL is typically in the range of 1 μg-200 μg, such as 10-100 μg, or 10 μg-50 μg per dose. An infant dose is typically at the lower end of this range, e.g., from about 1 μg to about 50 μg, such as from about 2 μg, or about 5 μg, or about 10 μg, to about 25 μg, or to about 50 μg. Typically, where QS21 is used in the formulation, the ranges are comparable (and according to the ratios indicated above). For adult and elderly populations, the formulations typically include more of an adjuvant component than is typically found in an infant formulation. In particular formulations using an oil-in-water emulsion, such an emulsion can include additional components, for example, such as cholesterol, squalene, alpha tocopherol, and/or a detergent, such as tween 80 or span85. In exemplary formulations, such components can be present in the following amounts: from about 1-50 mg cholesterol, from 2 to 10% squalene, from 2 to 10% alpha tocopherol and from 0.3 to 3% tween 80. Typically, the ratio of squalene: alpha tocopherol is equal to or less than 1 as this provides a more stable emulsion. In some cases, the formulation can also contain a stabilizer. Where alum is present, e.g., in combination with 3D-MPL, the amount is typically between about 100 μg and 1 mg, such as from about 100 μg, or about 200 μg to about 750 μg, such as about 500 μg per dose.

An immunogenic composition typically contains an immunoprotective quantity (or a fractional dose thereof) of the antigen and can be prepared by conventional techniques. Preparation of Immunogenic Compositions, Including Those for Administration to Human Subjects, is generally described in Pharmaceutical Biotechnology, Vol. 61 Vaccine Design—the subunit and adjuvant approach, edited by Powell and Newman, Plenum Press, 1995. New Trends and Developments in Vaccines, edited by Voller et al., University Park Press, Baltimore, Md., U.S.A. 1978. Encapsulation within liposomes is described, for example, by Fullerton, U.S. Pat. No. 4,235,877. Conjugation of proteins to macromolecules is disclosed, for example, by Likhite, U.S. Pat. No. 4,372,945 and by Armor et al., U.S. Pat. No. 4,474,757.

Typically, the amount of protein in each dose of the immunogenic composition is selected as an amount which induces an immunoprotective response without significant, adverse side effects in the typical subject. Immunoprotective in this context does not necessarily mean completely protective against infection; it means protection against symptoms or disease, especially severe disease associated with the virus. The amount of antigen can vary depending upon which specific immunogen is employed. Generally, it is expected that each human dose will comprise 1 1000 μg of protein, such as from about 1 μg to about 100 μg, for example, from about 1 μg to about 50 μg, such as about 1 μg, about 2 μg, about 5 μg, about 10 μg, about 15 μg, about 20 μg, about 25 μg, about 30 μg, about 40 μg, or about 50 μg. The amount utilized in an immunogenic composition is selected based on the subject population (e.g., infant or elderly). An optimal amount for a particular composition can be ascertained by standard studies involving observation of antibody titres and other responses in subjects. Following an initial vaccination, subjects can receive a boost in about 4 weeks.

### EXAMPLES

### Example 1

### Exemplary Chimeric RSV F2GF1 Polypeptide Antigens

Eight exemplary chimeric F2GF1 polypeptides were constructed based on the combination of three different variant domains. These eight variant F2GF1 polypeptides are illustrated in FIG. 2, and detailed below.

F2GF1-1 (P3-1). This exemplary chimeric F2GF1 polypeptide is 603 amino acids in length, and includes in an N-terminal to C-terminal orientation: amino acids 24-130 of the F2 domain; amino acids 128-229 of a G protein variant that has a single amino acid substitution of alanine in the place or asparagines at position 191; and amino acids 161-524 of the F1 domain. Between each of the segments (F2-G and G-F1) is introduced a 6 nucleotide linker encoding two glycines residues.

F2GF1-2 (P3-2). This exemplary chimeric F2GF1 polypeptide is 559 amino acids in length, and includes in an N-terminal to C-terminal orientation: amino acids 24-107 of the F2 domain; amino acids 149-229 of a G protein variant that has a single amino acid substitution of alanine in the place or asparagines at position 191; and amino acids 161-524 of the F2 domain. Between each of the segments (F2-G and G-F1) is introduced a 6 nucleotide linker encoding two glycines residues. An internal transcription start has been modified to optimize the production of the 559 amino acids full length product.

F2GF1-3 (P3-3). This exemplary chimeric F2GF1 polypeptide is 580 amino acids in length, and includes in an N-terminal to C-terminal orientation: amino acids 24-107 of the F2 domain; amino acids 129-229 of a G protein variant that has a single amino acid substitution of alanine in the place or asparagines at position 191; and amino acids 161-524 of the F2 domain. Between each of the segments (F2-G and G-F1) is introduced a 6 nucleotide linker encoding two glycines residues.

F2GF1-4 (P3-4). This exemplary chimeric F2GF1 polypeptide is 582 amino acids in length, and includes in an N-terminal to C-terminal orientation: amino acids 24-130 of the F2 domain; amino acids 149-229 of a G protein variant that has a single amino acid substitution of alanine in the place or asparagines at position 191; and amino acids 161-524 of the F2 domain. Between each of the segments (F2-G and G-F1) is introduced a 6 nucleotide linker encoding two glycines residues.

F2GF1-5 (P3-5). This exemplary chimeric F2GF1 polypeptide is similar to P3-1, except that the G polypeptide includes the naturally occurring asparagines at position 191. An internal transcription start has been modified to optimize the production of the 603 amino acids full length product.

F2GF1-6 (P3-6). This exemplary chimeric F2GF1 polypeptide is similar to P3-2, except that the G polypeptide includes the naturally occurring asparagines at position 191.

F2GF1-7 (P3-7). This exemplary chimeric F2GF1 polypeptide is similar to P3-3, except that the G polypeptide includes the naturally occurring asparagines at position 191.

F2GF1-8 (P3-8). This exemplary chimeric F2GF1 polypeptide is similar to P3-4, except that the G polypeptide includes the naturally occurring asparagines at position 191.

Exemplary Eukaryotic F2GF1 polypeptide. Exemplary eukaryotic chimeric F2GF1 polypeptides were produced to be similar in design to the F2GF1-2 and F2GF1-6 constructs designed above for prokaryotic expression. It will be understood that any of the variants described above can also be produced in the context of the eukaryotic vectors described herein. The eukaryotic version included the F0 native signal sequence, whereas the prokaryotic constructs described above do not possess a secretion signal. Incorporation of a signal sequence enhances post-translational modifications, such as glycosylation. In exemplary embodiments, one or both furin recognition motifs are removed. In addition, the G and F1 boundaries are slightly different from those of the prokaryotic constructs described above. The G peptide domain includes amino acids 152-229, instead of aa149-229 for the prokaryotic versions, and the F1 domain includes amino acids 151-524, instead of 161-524 present in the prokaryotic versions. Thus, this exemplary eukaryotic chimeric F2GF1 polypeptide includes the following sequence. From the N-terminus, the chimeric polypeptide includes amino acids 1-109 of the F0 polypeptide (including the signal peptide, the F2 domain and the first furin cleavage motif). There is a glycine linker at amino acid 110, followed by amino acids 152-229 of the G protein (either naturally occurring, or incorporating a substitution of alanine in the place of asparagines at position 191) at positions 111-188. Following the G protein domain at positions 189-562 are amino acids 151-524 of the F1 domain. Thus, in this variant, the native pep27, fusion peptide and one or both furin recognition motifs are replaced by the G protein domain.

This exemplary recombinant protein was designed to be expressed in mammalian Chinese Hamster Ovary (CHO) cells using a GS expression system. CHO cells grown in glutamine-free medium require exogenous glutamine for optimal growth. Following transfection of CHO cells with a pEE14 vector including a polynucleotide sequence encoding a chimeric F2GF1 polypeptide, this system enables selection of stable clones via metabolic deprivation, due to expression of glutamine synthase by the pEE14 vector. Although the constructs described here were produced for expression in CHO cells, these constructs can equally be produced for expression using a Baculovirus Expression Vector System (BEVS). The constructs (coding regions) made for CHO were codon optimized for better translation efficiency in BEVS but the amino acid sequence were kept identical to their CHO homologue. In the BEVS, the RSV optimized genes are cloned in the shuttle vector pAcSG2. That plasmid is used alone with a linearized Baculovirus genomic sequence to co-transfect insect cells. Specific recombination events occur in the cells and generate the recombinant baculovirus. During the infection process, the gene of interest is expressed at a very late stage under the polyhedrin promoter.

### Example 2

### Neutralization Inhibition in Human Sera by Chimeric F2GF1 Polypeptides

Human sera obtained from volunteer donors were screened for reactivity against RSV A by ELISA, and used in the neutralization inhibition (NI) assay at relevant dilution based on prior RSV neutralization potential titration. Sera were mixed with exemplary chimeric F2GF1 polypeptides, P3-1, P3-2, P3-3, P3-4 or chimeric FG antigen at concentrations of 0, 2, 10 and 25 μg/ml and incubated 1.5 to 2 hours at 37° C. In a round bottom 96-well plate, sera and proteins were mixed with a fixed concentration of RSV A and incubated for 20 min at 33° C.

The sera-inhibitor-virus mixtures was then placed into flat bottom 96-well plates previously seeded with Vero cells, and further incubated for 5-6 days at 33° C. with 5% CO₂ until immunofluorescence assay for NI titer detection.

Titers were calculated using the Reed-Muench method and percentages of NI calculated according to the following formula:

(NI titer of 25 μg/ml inhibitor-NI titer of 0 μg/ml inhibitor)÷NI titer of 0 μg/ml inhibitor×100.

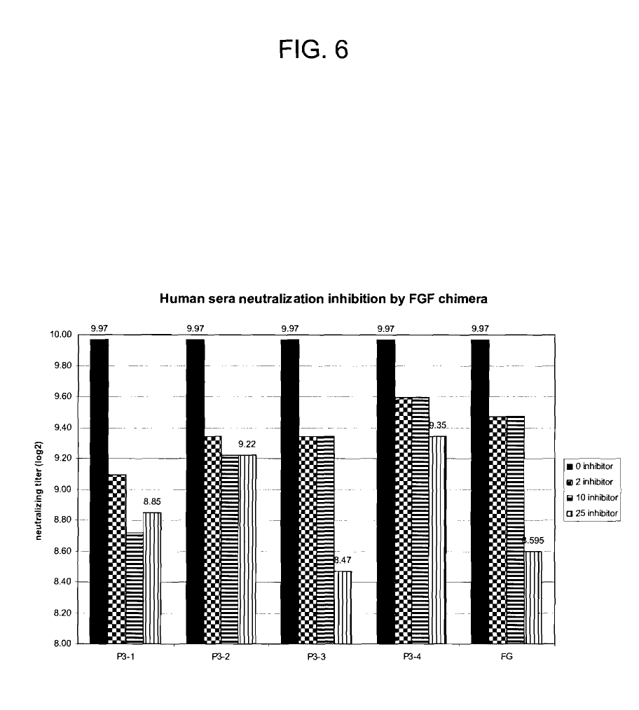

The exemplary results shown in FIG. 6 demonstrate that preF is superior to FG in NI in 11/14 donor tested and equal in the remaining three donors.

### Example 3

### Chimeric F2GF1 Protects Against Challenge with RSV

Mice were immunized with an immunogenic composition containing F2GF1 polypeptide and an adjuvant comprising MPL and QS21 in a liposomal formulation. Groups of mice were immunized three times at two week intervals with 2 μg of chimeric F2GF1 polypeptides (P3-2, P3-3, P3-6 and P3-7) and challenged three weeks after the third IM injection. Infection was assessed by titrating live virus present in lung homogenates four days after challenge.

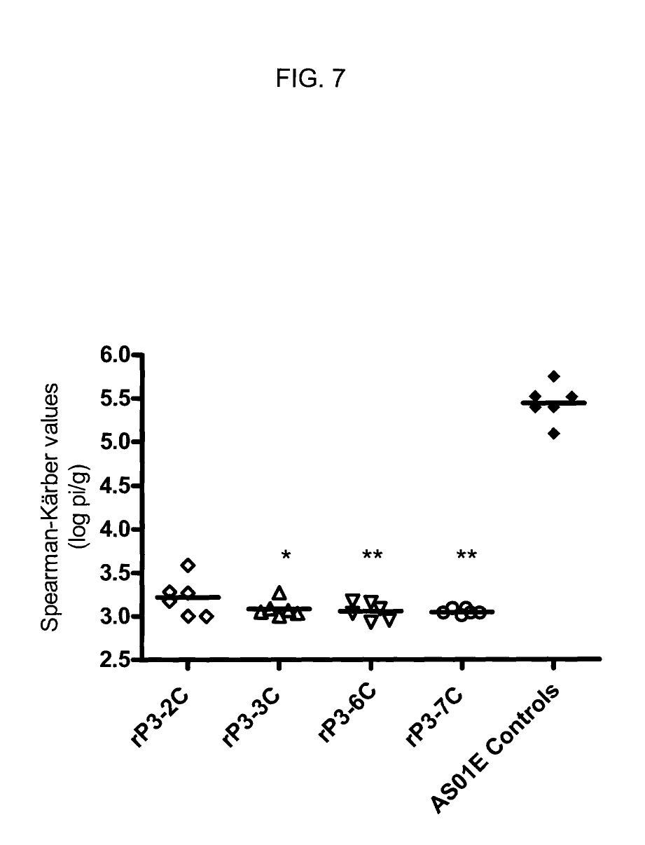

As shown in FIG. 7, three doses of an immunogenic composition containing 2 μg of F2GF1 antigen, in combination with adjuvant, elicit significant protection against RSV challenge as compared to control mice that received only adjuvant.

### Example 4

### Production of Neutralizing Antibodies Following Immunization with Chimeric F2GF1 Antigens

Mice were immunized three times at two weeks interval with 2 μg of F2GF1 (rP3-2, rP3-3, rP3-6 and rP3-7) and challenged three weeks after the third IM injection, as indicated above. Serum was collected immediately before challenge to quantitate production of neutralizing antibodies specific for RSV.

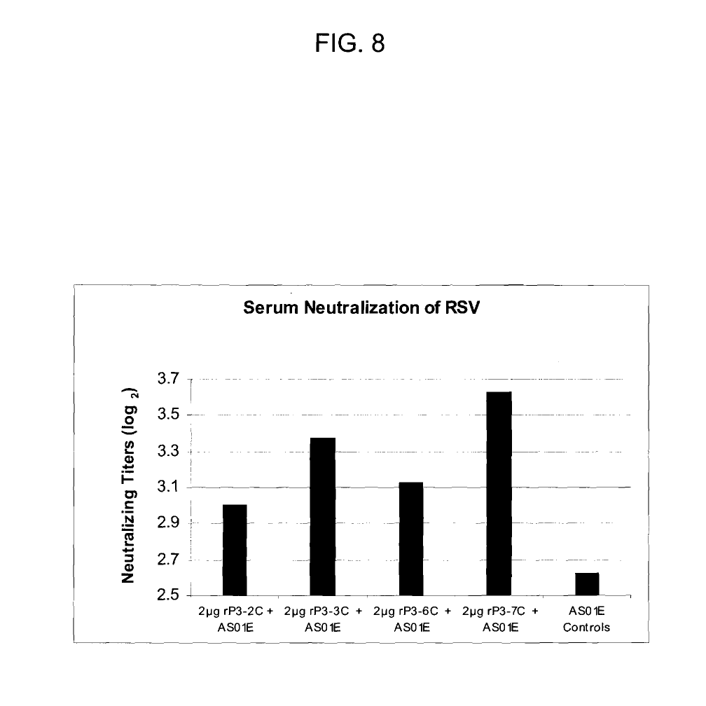

Sera of immunized mice were diluted serially and placed in the presence of fixed amounts of RSV to evaluate neutralizing activity of anti-RSV antibodies. Neutralizing antibody titers were calculated using the Spearman-Karber method as modified by Finney. The results (illustrated in Table 2 and FIG. 8) demonstrate that superior neutralizing antibodies against RSV were detected in sera of animals immunized with rP3-3 and rP3-7.

TABLE 2
Neutralization titres elicited by immunization with exemplary
F2GF1 antigens

| | | Neutralizing |
|---|---|---|
| | | Titers |
| Group | Antigen | (log2) |
| 1 | P3-2 | 3.0000 |
| 2 | P3-3 | 3.3750 |
| 3 | P3-6 | 3.1250 |
| 4 | P3-7 | 3.6250 |
| 5 | Adjuvant only | 2.6250 |

We claim:

1-79. (canceled)

80. A chimeric respiratory syncytial virus (RSV) polypeptide comprising in an N terminal to C terminal direction:
(i) a first F protein polypeptide domain;
(ii) a G protein polypeptide domain; and
(iii) a second F protein polypeptide domain.

81. The chimeric RSV polypeptide of claim 80, wherein the first F protein polypeptide domain (i) comprises at least an amino acid subsequence of an F protein F2 domain.

82. The chimeric RSV polypeptide of claim 81, wherein the first F protein polypeptide domain (i) comprises an amino acid sequence from residue 24 to residue 107 of a native F protein polypeptide.

83. The chimeric RSV polypeptide of claim 80, further comprising a signal peptide.

84. The chimeric RSV polypeptide of claim. 80, which comprises at least one amino acid deletion, addition or substitution that eliminates a furin cleavage site present in a naturally occurring RSV F protein.

85. The chimeric RSV polypeptide of claim 80, wherein the second F protein polypeptide domain (iii) comprises at least an amino acid subsequence of an F protein F1 domain.

86. The chimeric RSV polypeptide of claim 80, wherein the second F protein polypeptide domain (iii) comprises an amino acid sequence from residue 161 to residue 524 of a native F protein polypeptide.

87. The chimeric RSV polypeptide of claim 80, wherein the second F protein polypeptide domain (iii) comprises an amino acid sequence from residue 151 to residue 524 of a native F protein polypeptide.

88. The chimeric RSV polypeptide of claim 80, wherein the G protein polypeptide domain (ii) comprises at least one immunodominant T-cell epitope of a native G protein polypeptide.

89. The chimeric RSV polypeptide of claim 80, wherein the immunodominant T-cell epitope comprises from amino acid residue 183 to residue 203 of a native G protein polypeptide.

90. The chimeric RSV polypeptide of claim 88, wherein the chimeric polypeptide comprises a substitution of asparagine by alanine at residue 191 (N191A) of the G protein.

91. A recombinant RSV antigen comprising a multimer of the chimeric RSV polypeptides of any of the preceding claims.

92. The recombinant RSV antigen of claim 91, wherein the RSV antigen comprises a trimer of chimeric polypeptides.

93. An immunogenic composition comprising an antigen comprising the chimeric RSV polypeptide of claim 80, and a carrier or excipient.

94. The immunogenic composition of claim 93, further comprising an adjuvant.

95. The immunogenic composition of claim 94, wherein the adjuvant is a Th1-biasing adjuvant.

96. The immunogenic composition of claim 93, further comprising at least one additional antigen of a pathogenic organism other than RSV.

97. A recombinant nucleic acid comprising a polynucleotide sequence that encodes the chimeric polypeptide of claim 80.

98. A host cell comprising the nucleic acid of claim 97.

99. A method for eliciting an immune response against Respiratory Syncytial Virus (RSV), the method comprising:
administering to a subject an immunogenically effective amount of a composition comprising an antigen comprising the chimeric RSV polypeptide of claims 80.

100. The method of claim 99, wherein administering the composition comprising the chimeric RSV polypeptide elicits an immune response specific for RSV without enhancing viral disease following contact with RSV.

101. The method of claim 100, wherein the immune response comprises a Th1-type immune response.
